(19) United States
(12) Patent Application Publication
(10) Pub. No.: US 2013/0075705 A1
(43) Pub. Date: Mar. 28, 2013

(54) Carbazole Compound, Light-Emitting Element Material, and Organic Semiconductor Material

(71) Applicants: Takako TAKASU, Chigasaki (JP); Nobuharu OHSAWA, Tochigi (JP)

(72) Inventors: Takako TAKASU, Chigasaki (JP); Nobuharu OHSAWA, Tochigi (JP)

(73) Assignee: Semiconductor Energy Laboratory Co., Ltd., Kanagawa-ken (JP)

(21) Appl. No.: 13/611,346

(22) Filed: Sep. 12, 2012

(30) Foreign Application Priority Data
Sep. 22, 2011 (JP) 2011-207410

(51) Int. Cl. C07D 403/14; H01L 51/54

(52) U.S. Cl. 257/40; 548/305.1; 257/E51.026

(57) ABSTRACT

A carbazole compound which can be used for a transport layer or as a host material or a light-emitting material of a light-emitting element is provided. Specifically, a carbazole compound which makes it possible to obtain a light-emitting element having good characteristics when used in a light-emitting element emitting blue phosphorescence is provided. In the carbazole compound, the 9-position of one carbazole, the 9-position of the other carbazole, and the 1-position of a benzimidazole skeleton are bonded to the 1-position, the 3-position, and the 5-position of benzene.

### BACKGROUND OF THE INVENTION

1. Field of the Invention

The present invention relates to a carbazole compound which can be used as a light-emitting element material. The present invention also relates to a light-emitting element material and an organic semiconductor material each of which uses the carbazole compound.

2. Description of the Related Art

As next generation lighting devices or display devices, display devices using light-emitting elements (organic EL elements) in which organic compounds are used for light-emitting substances have been developed rapidly because of their advantages of such as thinness, lightweightness, high speed response to input signals, low power consumption, and the like.

In an organic EL element, voltage application between electrodes between which a light-emitting layer is provided causes recombination of electrons and holes injected from the electrodes, which brings a light-emitting substance into an excited state, and the return from the excited state to the ground state is accompanied by light emission. Since the wavelength of light emitted from a light-emitting substance is peculiar to the light-emitting substance, use of different types of organic compounds for light-emitting substances makes it possible to provide light-emitting elements which exhibit various wavelengths, i.e., various colors.

In the case of display devices which are expected to display images, such as displays, at least three-color light, i.e., red light, green light, and blue light are necessary for reproduction of full-color images. Further, in application to lighting devices, light having wavelength components evenly spreading in the visible light region is ideal for obtaining a high color rendering property, but actually, light obtained by mixing two or more kinds of light having different wavelengths is often used for lighting application. Note that it is known that mixing light of three colors of red, green, and blue allows generation of white light having a high color rendering property.

Light emitted from a light-emitting substance is peculiar to the substance as described above. However, important performances as a light-emitting element, such as lifetime, power consumption, and even emission efficiency, are not only dependent on the light-emitting substance but also greatly dependent on layers other than the light-emitting layer, an element structure, properties of an emission center substance and a host material, compatibility between them, carrier balance, and the like. Therefore, there is no doubt that many kinds of light-emitting element materials are necessary for a growth in this field. For the above-described reasons, light-emitting element materials with a variety of molecular structures have been proposed (e.g., see Patent Document 1).

As is generally known, the generation ratio of a singlet excited state to a triplet excited state in a light-emitting element using electroluminescence is 1:3. Therefore, a light-emitting element in which a phosphorescent material capable of converting the triplet excited state to light emission is used as an emission center substance can theoretically realize higher emission efficiency than a light-emitting element in which a fluorescent material capable of converting the singlet excited state to light emission is used as an emission center substance.

However, since the triplet excited state of a substance is at a lower energy level than the singlet excited state of the substance, a substance that emits phosphorescence can be said to have a wider band gap than a substance that emits fluorescence when the emissions of the substances are at the same wavelength.

As a host material in a host-guest type light-emitting layer or a substance contained in each transport layer in contact with a light-emitting layer, a substance having a wider band gap or a higher triplet excitation level (a larger energy difference between a triplet excited state and a singlet ground state) than an emission center substance is used for efficient conversion of excitation energy into light emission from the emission center substance.

Therefore, a host material and a carrier-transport material each having a further wider band gap are necessary in order that light emission having a shorter wavelength than blue fluorescence or green phosphorescence be efficiently obtained. It is very difficult to develop a substance to be a light-emitting element material which has such a wide band gap while enabling a balance between important characteristics of a light-emitting element, such as low driving voltage, high emission efficiency, and a long lifetime.

### REFERENCE

### [Patent Document 1] Japanese Published Patent Application No. 2007-15933

### SUMMARY OF THE INVENTION

In view of the above, an object of one embodiment of the present invention is to provide a carbazole compound which can be used for a transport layer or as a host material or a light-emitting material of a light-emitting element. Specifically, an object of one embodiment of the present invention is to provide a carbazole compound which makes it possible to obtain a light-emitting element having good characteristics when used in a light-emitting element emitting blue phosphorescence.

Another object of one embodiment of the present invention is to provide a carbazole compound which has a high T₁ level. Specifically, the object of one embodiment of the present invention is to provide a carbazole compound which makes it possible to obtain a light-emitting element having high emission efficiency when used in a light-emitting element emitting blue phosphorescence.

Another object of one embodiment of the present invention is to provide a carbazole compound which has a high carrier-transport property. Specifically, the object of one embodiment of the present invention is to provide a carbazole compound which can be used in a light-emitting element emitting blue phosphorescence and allows the driving voltage of the light-emitting element to be low.

Another object of one embodiment of the present invention is to provide a light-emitting element material using the carbazole compound.

Another object of one embodiment of the present invention is to provide a light-emitting element using the carbazole compound.

Another object of one embodiment of the present invention is to provide a light-emitting device, a lighting device, a display device, and an electronic device each using the carbazole compound.

It is only necessary that at least one of the above-described objects be achieved in the present invention.

One embodiment of the present invention is a carbazole compound in which the 9-position of one carbazole skeleton, the 9-position of the other carbazole skeleton, and the 1-position of a benzimidazole skeleton are bonded to the 1-position, the 3-position, and the 5-position of benzene. The use of the carbazole compound as a light-emitting element material allows a light-emitting element having good characteristics to be obtained.

The carbazole compound includes the benzimidazole skeleton which is an electron-transport skeleton and the carbazole skeletons which are hole-transport skeletons, so that the carbazole compound has a high carrier-transport property. Further, these two kinds of carrier-transport skeletons are bonded through the benzene skeleton, so that the carbazole compound has a wide band gap and a high T₁ level.

Specifically, one embodiment of the present invention is a carbazole compound represented by General Formula (G1).

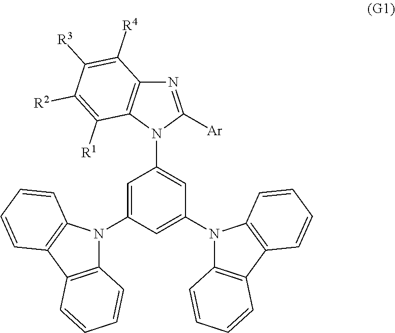

Note that in the formula, Ar represents an aryl group having 6 to 18 carbon atoms, and R¹ to R⁴ separately represent any of hydrogen, an alkyl group having 1 to 4 carbon atoms, and an aryl group having 6 to 13 carbon atoms.

Note that an alkyl group is not bonded to a carbazole skeleton in the carbazole compound. This allows synthesis to be easily performed and the carbazole compound to be easily deposited by evaporation. Therefore, the carbazole compound of one embodiment of the present invention is very suitable as a material for a light-emitting element manufactured by evaporation.

In the carbazole compound of one embodiment of the present invention, the benzene to which the carbazole is bonded is bonded at the 1-position of the benzimidazole skeleton as described above; thus, the carbazole compound has a higher T₁ level than a substance in which the benzene is bonded at the 2-position of a benzimidazole skeleton. Further, in the carbazole compound of one embodiment of the present invention, the carrier-transport skeletons such as the carbazole skeletons and the benzimidazole skeleton are bonded at the 1-position, the 3-position, and the 5-position of the benzene skeleton; thus, the carbazole compound keeps a high T₁ level. Moreover, in the carbazole compound of one embodiment of the present invention, the carbazole skeleton is bonded to the benzene skeleton at the 9-position; thus, the carbazole compound has a high T₁ level.

In the carbazole compound of one embodiment of the present invention, the benzimidazole skeleton and the two carbazole skeletons are bonded to the benzene; thus, the carbazole compound has a sufficiently high molecular weight and a steric structure, and thus has high heat resistance and high glass transition temperature (Tg). Therefore, a film formed by deposition of the carbazole compound of one embodiment of the present invention by evaporation has good film quality.

In the carbazole compound represented by General Formula (G1), the group represented by Ar is preferably a phenyl group.

Another embodiment of the present invention is a carbazole compound represented by Structural Formula (100).

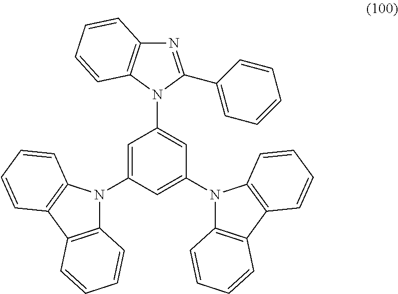 [SMILES: c1ccc(-c2nc3ccccc3n2-c2cc(-n3c4ccccc4c4ccccc43)cc(-n3c4ccccc4c4ccccc43)c2)cc1]

The carbazole compound represented by General Formula (G1) has a high carrier-transport property and thus can be suitably used as a host material or a carrier-transport material for a light-emitting element. In other words, another embodiment of the present invention is a light-emitting element material including the carbazole compound represented by General Formula (G1) or Structural Formula (100).

A light-emitting element manufactured using the carbazole compound having the above structure can have high emission efficiency and low driving voltage. In other words, another embodiment of the present invention is a light-emitting element which includes a layer containing an organic compound between a pair of electrodes. The carbazole compound is contained in the layer containing an organic compound. By application of current between the pair of electrodes, the light-emitting element emits light.

The carbazole compound is very suitable as a host material in a light-emitting element. In other words, another embodiment of the present invention is a light-emitting element which includes a layer containing an organic compound between a pair of electrodes. The layer containing an organic compound layer includes a light-emitting layer containing a host material and an emission center material. The carbazole compound is contained as the host material. By application of current between the pair of electrodes, the light-emitting element emits light.

The light-emitting element including the carbazole compound is used in a light-emitting device, so that the light-emitting device can have low power consumption. In other words, another embodiment of the present invention is a light-emitting device including the light-emitting element.

The light-emitting element including the carbazole compound is used in a lighting device, so that the lighting device can have low power consumption. In other words, another embodiment of the present invention is a lighting device including the light-emitting element.

The light-emitting element including the carbazole compound is used in a display device, so that the display device can have low power consumption. In other words, another embodiment of the preset invention is a display device including the light-emitting element.

The light-emitting element including the carbazole compound is used in an electronic device, so that the electronic device can have low power consumption. In other words, another embodiment of the present invention is an electronic device including the light-emitting element.

The carbazole compound having any of the above structures is a substance having both a high carrier-transport property and a wide energy gap, and can be suitably used for a material contained in a transport layer or a host material or an emission center substance in a light-emitting layer in a light-emitting element. A light-emitting element using a light-emitting element material containing the carbazole compound can be a light-emitting element having high emission efficiency. A light-emitting element including the carbazole compound can have low driving voltage. The carbazole compound can also be used as an organic semiconductor material.

### BRIEF DESCRIPTION OF THE DRAWINGS

FIGS. 1A and 1B are conceptual diagrams of light-emitting elements.

FIG. 2 is a conceptual diagram of an organic semiconductor element.

FIGS. 3A and 3B are conceptual diagrams of an active matrix light-emitting device.

FIGS. 4A and 4B are conceptual diagrams of a passive matrix light-emitting device.

FIGS. 5A to 5D each illustrate an electronic device.

FIG. 6 illustrates a light source device.

FIG. 7 illustrates a lighting device.

FIG. 8 illustrates lighting devices.

FIG. 9 illustrates in-vehicle display devices and lighting devices.

FIGS. 10A to 10C illustrate an electronic device.

FIGS. 11A and 11B are NMR charts of mCP-Cl.

FIGS. 12A and 12B are NMR charts of 1Cz2BIm.

FIGS. 13A and 13B each show an absorption and emission spectra of 1Cz2BIm.

FIG. 14 shows luminance versus current density characteristics of a light-emitting element 1 and a comparative light-emitting element 1.

FIG. 15 shows luminance versus voltage characteristics of the light-emitting element 1 and the comparative light-emitting element 1.

FIG. 16 shows current efficiency versus luminance characteristics of he light-emitting element 1 and the comparative light-emitting element 1.

FIG. 17 shows current versus voltage characteristics of the light-emitting element 1 and the comparative light-emitting element 1.

FIG. 18 shows power efficiency versus luminance characteristics of the light-emitting element 1 and the comparative light-emitting element 1.

FIG. 19 shows external quantum efficiency versus luminance characteristics of the light-emitting element 1 and the comparative light-emitting element 1.

FIG. 20 shows emission spectra of the light-emitting element 1 and the comparative light-emitting element 1.

FIG. 21 shows normalized luminance versus time characteristics of the light-emitting element 1 and the comparative light-emitting element 1.

FIG. 22 shows luminance versus current density characteristics of a light-emitting element 2 and a comparative light-emitting element 2.

FIG. 23 shows luminance versus voltage characteristics of the light-emitting element 2 and the comparative light-emitting element 2.

FIG. 24 shows current efficiency versus luminance characteristics of the light-emitting element 2 and the comparative light-emitting element 2.

FIG. 25 shows current versus voltage characteristics of the light-emitting element 2 and the comparative light-emitting element 2.

FIG. 26 shows power efficiency versus luminance characteristics of the light-emitting element 2 and the comparative light-emitting element 2.

FIG. 27 shows external quantum efficiency versus luminance characteristics of the light-emitting element 2 and the comparative light-emitting element 2.

FIG. 28 shows emission spectra of the light-emitting element 2 and the comparative light-emitting element 2.

FIG. 29 shows normalized luminance versus time characteristics of the light-emitting element 2 and the comparative light-emitting element 2.

FIG. 30 shows luminance versus current density characteristics of a light-emitting element 3 and a comparative light-emitting element 3.

FIG. 31 shows luminance versus voltage characteristics of the light-emitting element 3 and the comparative light-emitting element 3.

FIG. 32 shows current efficiency versus luminance characteristics of the light-emitting element 3 and the comparative light-emitting element 3.

FIG. 33 shows current versus voltage characteristics of the light-emitting element 3 and the comparative light-emitting element 3.

FIG. 34 shows power efficiency versus luminance characteristics of the light-emitting element 3 and the comparative light-emitting element 3.

FIG. 35 shows external quantum efficiency versus luminance characteristics of the light-emitting element 3 and the comparative light-emitting element 3.

FIG. 36 shows emission spectra of the light-emitting element 3 and the comparative light-emitting element 3.

FIG. 37 shows normalized luminance versus time characteristics of the light-emitting element 3 and the comparative light-emitting element 3.

FIG. 38 shows luminance versus current density characteristics of a light-emitting element 4 and a comparative light-emitting element 4.

FIG. 39 shows luminance versus voltage characteristics of the light-emitting element 4 and the comparative light-emitting element 4.

FIG. 40 shows current efficiency versus luminance characteristics of the light-emitting element 4 and the comparative light-emitting element 4.

FIG. 41 shows current versus voltage characteristics of the light-emitting element 4 and the comparative light-emitting element 4.

FIG. 42 shows power efficiency versus luminance characteristics of the light-emitting element 4 and the comparative light-emitting element 4.

FIG. 43 shows external quantum efficiency versus luminance characteristics of the light-emitting element 4 and the comparative light-emitting element 4.

FIG. 44 shows emission spectra of the light-emitting element 4 and the comparative light-emitting element 4.

FIG. 45 shows normialized luminance versus time characteristics of the light-emitting element 4 and the comparative light-emitting element 4.

### DETAILED DESCRIPTION OF THE INVENTION

Hereinafter, embodiments of the present invention will be described. It is easily understood by those skilled in the art that modes and details disclosed herein can be modified in various ways without departing from the spirit and scope of the present invention. Therefore, the present invention is not construed as being limited to description of the embodiments.

### Embodiment 1

In a carbazole compound of this embodiment, the 9-position of one carbazole skeleton, the 9-position of the other carbazole skeleton, and the 1-position of a benzimidazole skeleton are bonded to the 1-position, the 3-position, and the 5-position of benzene. Further, by using the carbazole compound as a light-emitting element material, a light-emitting element having good characteristics can be obtained.

The carbazole compound includes the benzimidazole skeleton which is an electron-transport skeleton and the carbazole skeletons which are hole-transport skeletons, so that the carbazole compound has a high carrier-transport property. Further, these two kinds of carrier-transport skeletons are bonded through the benzene skeleton, so that the carbazole compound has a wide band gap and a high T₁ level.

As an example of an aryl group bonded at the 2-position of the benzimidazole skeleton, an aryl group having 6 to 18 carbon atoms can be given. As the aryl group having 6 to 18 carbon atoms, specifically, a phenyl group, a biphenyl group, a terphenyl group, or the like can be used. Note that in the case where the aryl group is a biphenyl group, a meta-substituted biphenyl group is preferable to a para-substituted biphenyl group for a high triplet excitation level of the carbazole compound.

The carbazole compound having such a structure has a wide band gap and thus can be suitably used as a host material in which a substance emitting fluorescence or phosphorescence having a wavelength equal to or longer than that of blue light is dispersed in a light-emitting layer of a light-emitting element. Since the carbazole compound has a wide band gap, which means a high triplet excitation level (T₁ level), the energy of carriers that have recombined in a host material can be effectively transferred to an emission center substance. Thus, a light-emitting element having high emission efficiency can be manufactured.

Further, the carbazole compound having a wide band gap can prevent deactivation due to transfer of excitation energy of a light-emitting substance to a carrier-transport layer, and thus can also be suitably used for a carrier-transport layer adjacent to a light-emitting layer. Thus, a light-emitting element having high emission efficiency can be manufactured.

The carbazole compound can be suitably used as a host material or for a carrier-transport layer in a light-emitting element also because it has a high carrier-transport property. Owing to the high carrier-transport property of the carbazole compound, a light-emitting element having low driving voltage can be manufactured.

In the carbazole compound, the carbazole skeleton preferably has no substituent such as an alkyl group, in which case the number of synthesis steps can be reduced. When the carbazole skeleton does not have a substituent such as an alkyl group, the carbazole compound can be easily deposited by evaporation. Accordingly, a light-emitting element with more stable quality can be provided. Further, a light-emitting element including such a carbazole compound tends to have a long lifetime.

Further, when R¹ to R⁴ are each hydrogen, synthesis can be performed easily, which means that the carbazole compound is suitable for mass production.

The above-described carbazole compound can also be represented by General Formula (G1).

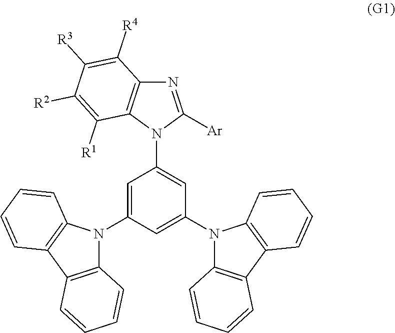

In the formula, R¹ to R⁴ separately represent any of hydrogen, an alkyl group having 1 to 4 carbon atoms, and an aryl group having 6 to 13 carbon atoms. The aryl group may further have a⁻substituent.

In the formula, Ar represents an aryl group having 6 to 18 carbon atoms, and the aryl group may further have a substituent.

Specific examples of the alkyl group having 1 to 4 carbon atoms which is represented by R¹ to R⁴ are a methyl group, an ethyl group, a propyl group, an isopropyl group, a butyl group, a sec-butyl group, an isobutyl group, a tert-butyl group, and the like. Specific examples of the aryl group having 1 to 13 carbon atoms are a phenyl group, a naphthyl group, a biphenyl group, a fluorenyl group, and the like. Note that in the case where the aryl group further has a substituent, specific examples of the substituent are an alkyl group having 1 to 6 carbon atoms, specifically, a methyl group, an ethyl group, a tert-butyl group, a cyclohexyl group, and the like.

Note that R¹ to R⁴ are preferably hydrogen, in which case synthesis is performed easily.

Specifically, a phenyl group, a biphenyl group, or a terphenyl group can be applied to Ar. More specifically, groups represented by Structural Formulae (Ar-1) to (Ar-5) are preferable.

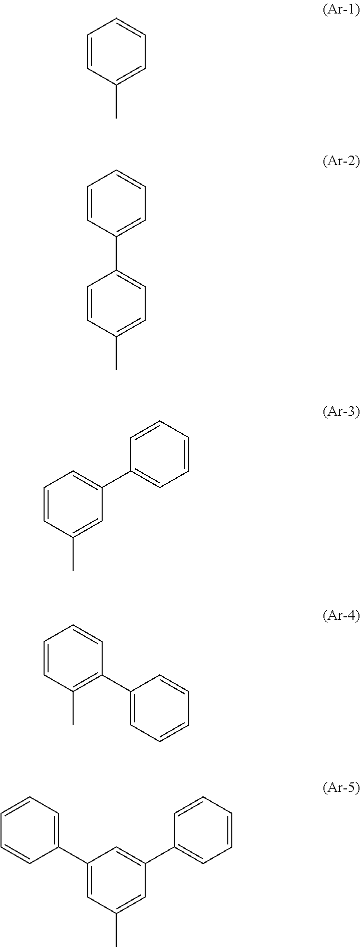

In the carbazole compound represented by General Founula (G1), the group represented by Ar is preferably a phenyl group. When Ar is a phenyl group, the carbazole compound can have a higher T₁ level.

In the case where Ar is a biphenyl group, a meta-substituted biphenyl group is preferable to a para-substituted biphenyl group for a high triplet excitation level of the carbazole compound. In other words, a group represented by Structural Formula (Ar-3) is preferable in the case where Ar is a biphenyl group.

Note that an alkyl group is not bonded to the carbazole skeleton in the carbazole compound represented by General Formula (G1). This allows synthesis to be easily performed and the carbazole compound to be easily deposited by evaporation. Therefore, the carbazole compound of one embodiment of the present invention is very suitable as a material for a light-emitting element manufactured by evaporation.

In the carbazole compound represented by General Formula (GI), the benzene to which the carbazole is bonded is bonded at the 1-position of the benzimidazole skeleton as described above; thus, the carbazole compound has a higher T₁ level than a substance in which the benzene is bonded at the 2-position of a benzimidazole skeleton. Further, in the carbazole compound, the carrier-transport skeletons such as the carbazole skeletons and the benzimidazole skeleton are bonded at the 1-position, the 3-position, and the 5-position of the benzene skeleton; thus, the carbazole compound keeps a high T₁ level. Moreover, in the carbazole compound, the carbazole skeleton is bonded to the benzene skeleton at the 9-position; thus, the carbazole compound has a high T₁ level.

In the carbazole compound of one embodiment of the present invention, the benzimidazole skeleton and the two carbazole skeletons are bonded to the benzene; thus, the carbazole compound has a sufficiently high molecular weight and a steric structure, and thus has high heat resistance and high glass transition temperature (Tg). Therefore, a film formed by deposition of the carbazole compound of one embodiment of the present invention by evaporation has good film quality.

Specific examples of the carbazole compound represented by General Formula (G1) are substances represented by Structural Formulae (100) to (104), and the like.

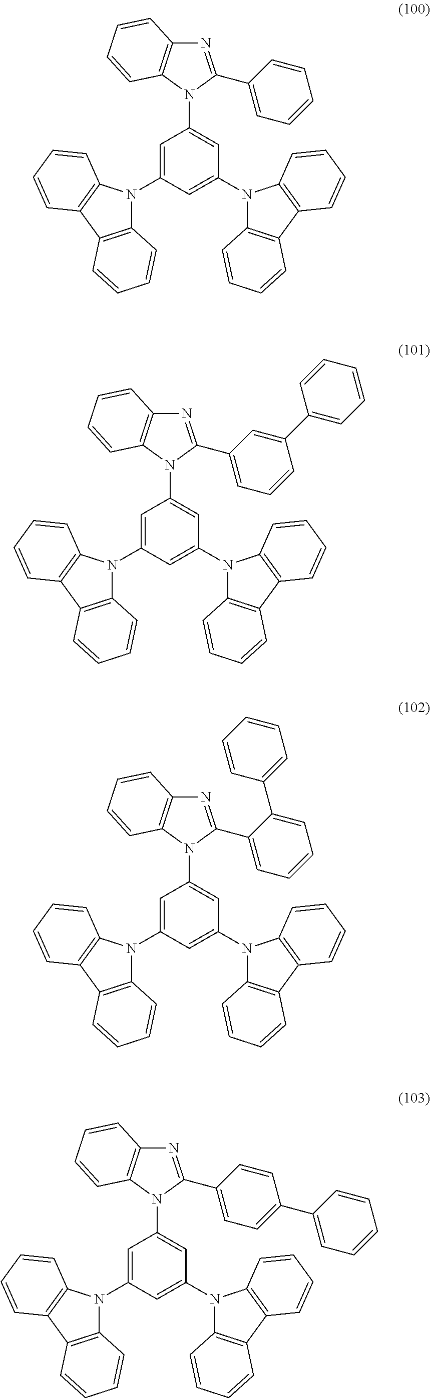

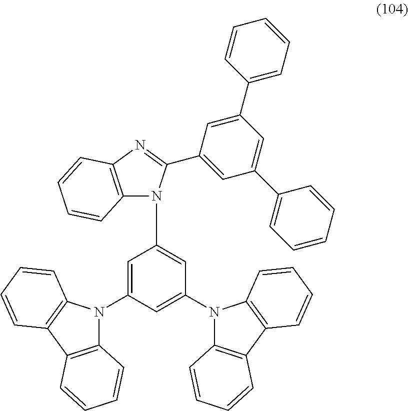

The above-described carbazole compound has a high carrier-transport property and thus is suitable as a carrier-transport material or a host material. Thus, a light-emitting element having low driving voltage can also be provided. Further, the carbazole compound has a- high triplet excitation level (a large energy difference between a triplet excited state and a ground state), so that a phosphorescent light-emitting element having high emission efficiency can be obtained. Moreover, the high triplet excitation level corresponds to a wide band gap; thus, the carbazole compound enables even a light-emitting element emitting blue fluorescence to efficiently emit light.

Further, the carbazole compound in this embodiment can also be used as a light-emitting material which emits blue to ultraviolet light.

Next, a synthesis method of the carbazole compound represented by General Formula (G1) is described.

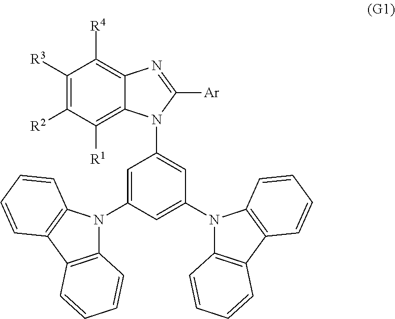

A variety of reactions can be applied to the synthesis method of the carbazole compound. For example, Synthesis Schemes (A-1) and (B-1) described below enable the synthesis of the carbazole compound represented by General Formula (G1). In General Formula (G1), R¹ to R⁴ separately represent any of hydrogen, an alkyl group having 1 to 4 carbon atoms, and an aryl group having 6 to 13 carbon atoms. The aryl group may further have a substituent. Ar represents an aryl group having 6 to 18 carbon atoms, and the aryl group may further have a substituent. Specific examples of the substituents are already given; therefore, the description is omitted here.

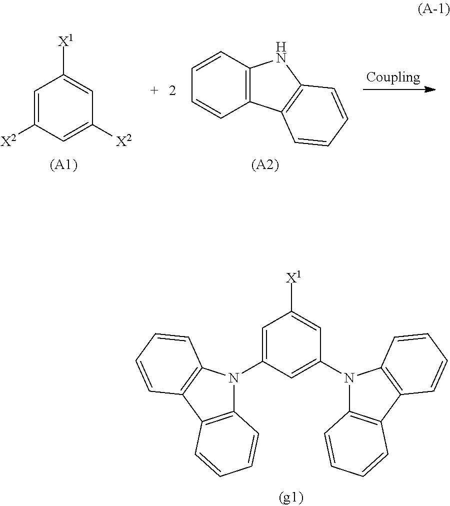

In Synthesis Scheme (A-1), X¹ and X² separately represent a halogeno group or a triflate group. Specific examples of the halogeno group are iodine, bromine, chlorine, and the like. It is preferable that X² have higher reactivity than X¹. Iodine, bromine, and chlorine, which are halogens, are ranked in order of reactivity.

In the synthesis method represented by Synthesis Scheme (A-1), a carbazole compound represented by General Formula (g1) is obtained by coupling an aryl compound (Al) having a halogeno group with 9H-carbazole (A2).

There are a variety of reaction conditions for the coupling reaction of the aryl compound (Al) having a halogen group with the 9-position of the carbazole (A2), which is shown in Synthesis Scheme (A-1). As an example of the reaction condition, the Buchwald-Hartwig reaction or the Ullmann reaction, in which a metal catalyst is used in the presence of a base, can be employed.

In the Buchwald-Hartwig reaction, a palladium catalyst can be used as a metal catalyst, and a mixture of a palladium complex and a ligand thereof can be used as the palladium catalyst. Examples of the palladium complex are bis(dibenzylideneacetone)palladium(0), palladium(II) acetate, and the like. Examples of the ligand are tri(tert-butyl)phosphine, tri(n-hexyl)phosphine, tricyclohexylphosphine, 1,1-bis(diphenylphosphino)ferrocene (abbreviation: DPPF), and the like. Examples of the substance that can be used as the base are an organic base such as sodium tert-butoxide, an inorganic base such as potassium carbonate, and the like. The Buchwald-Hartwig reaction is preferably performed in a solution, and toluene, xylene, benzene, or the like can be used as the solvent. However, the catalyst, ligand, base, and solvent that can be used are not limited thereto. Note that the Buchwald-Hartwig reaction is preferably performed in an inert atmosphere of nitrogen, argon, or the like.

Further, in the case where the Ullmann reaction is employed as a coupling method for Synthesis Scheme (A-1), a copper catalyst (e.g., copper(I) iodide or copper(II) acetate) is used as the metal catalyst. An inorganic base such as potassium carbonate can be used as the base. The Ullmann reaction is preferably performed in a solution, and 1,3-dimethyl-3,4,5,6-tetrahydro-2(1H)-pyrimidinone (abbreviation: DMPU), toluene, xylene, benzene, or the like can be used as the solvent. However, the catalyst, base, and solvent that can be used are not limited thereto. The Ullmann reaction is preferably performed in an inert atmosphere of nitrogen, argon, or the like.

Note that a solvent having a high boiling point, such as DMPU or xylene, is preferably used because, in the Ullmann reaction, an object can be obtained in a shorter time and at a higher yield when the reaction temperature is higher than or equal to 100° C. In addition, the reaction temperature is more preferably higher than or equal to 150° C.; therefore, DMPU is more preferably used.

Next, the carbazole compound (g1) obtained in Synthesis Scheme (A-1) is coupled with a benzimidazole compound (B1), so that the carbazole compound represented by General Formula (G1) can be obtained (Synthesis Scheme (B-1)).

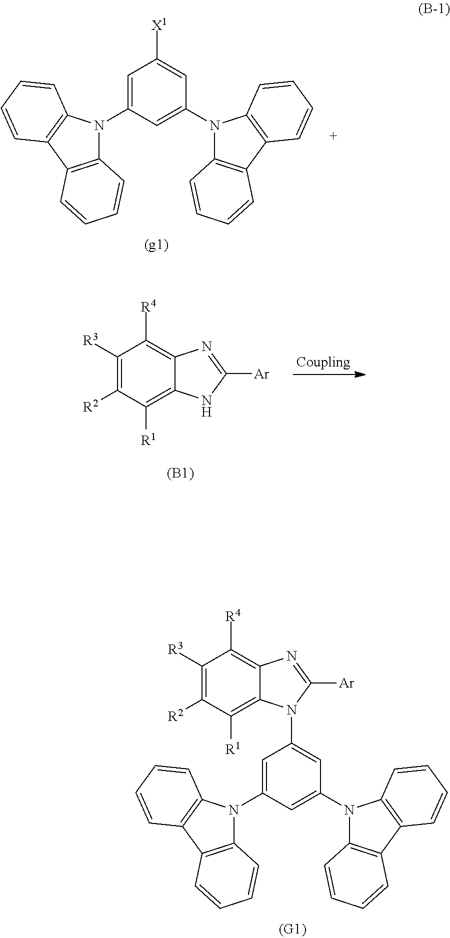

There are a variety of reaction conditions for the coupling reaction of the carbazole compound (g1) having a halogeno group with the 1-position of the benzimidazole compound (B1), which is shown in Synthesis Scheme (B-1). An example of the reaction condition is the Buchwald-Hartwig reaction or the Ullmann reaction, in which a metal catalyst is used in the presence of a base.

In the Buchwald-Hartwig reaction, a palladium catalyst can be used as a metal catalyst, and a mixture of a palladium complex and a ligand thereof can be used as the palladium catalyst. Examples of the palladium complex are bis(dibenzylideneacetone)palladium(0), palladium(II) acetate, allylpalladium(II)chloride dimer ([PdCl(C₃H₅)]₂), and the like. Examples of the ligand are tri(tert-butyl)phosphine, tri(n-hexyl)phosphine, tricyclohexylphosphine, 1,1-bis(diphenylphosphino)ferrocene (abbreviation: DPPF), di-tert-butyl(2,2-diphenyl-1-methyl-1-cyclopropyl)phosphine, and the like. Examples of the substance that can be used as the base are an organic base such as sodium tert-butoxide, an inorganic base such as potassium carbonate, and the like. In addition, this reaction is preferably performed in a solution, and examples of the solvent that can be used are toluene, xylene, benzene, and the like. However, the catalyst, ligand, base, and solvent that can be used are not limited thereto. Further, this reaction is preferably performed in an inert atmosphere of nitrogen, argon, or the like.

Further, in the case where the Ullmann reaction is employed as a coupling method for Synthesis Scheme (B-1), a copper catalyst (e.g., copper(I) iodide or copper(II) acetate) is used as the metal catalyst. An inorganic base such as potassium carbonate can be used as the base. This reaction is preferably performed in a solution, and 1,3-dimethyl-3,4,5,6-tetrahydro-2(1H)-pyrimidinone (abbreviation: DMPU), toluene, xylene, benzene, or the like can be used as the solvent. However, the catalyst, base, and solvent that can be used are not limited thereto. This reaction is preferably performed in an inert atmosphere of nitrogen, argon, or the like.

Note that a solvent having a high boiling point such as DMPU or xylene is preferably used because, in the Ullmann reaction, an object can be obtained in a shorter time and at a higher yield when the reaction temperature is higher than or equal to 100° C. In addition, the reaction temperature is more preferably higher than or equal to 150° C.; therefore, DMPU is more preferably used.

In this manner, the carbazole compound represented by General Formula (G1) can be synthesized.

### Embodiment 2

In this embodiment, an example will be described in which the carbazole compound described in Embodiment 1 is used for an active layer of a vertical transistor (SIT), which is a kind of an organic semiconductor element.

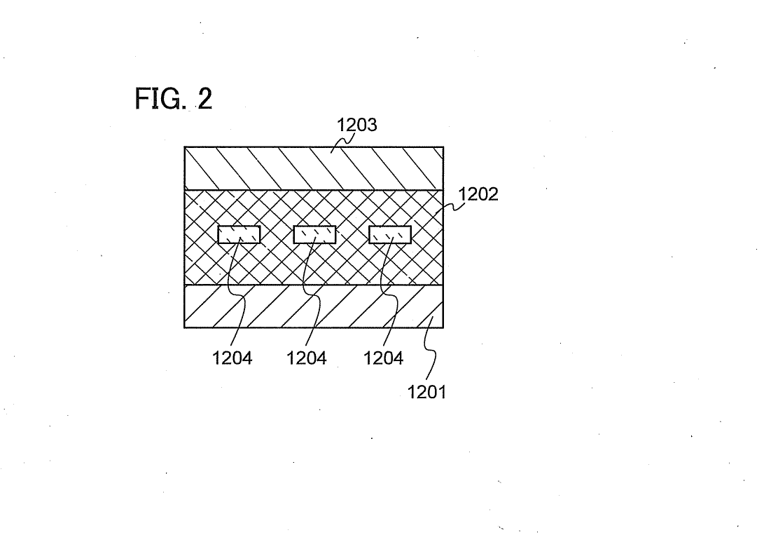

The element has a structure in which a thin-film active layer 1202 containing any of the carbazole compounds described in Embodiment 1 is interposed between a source electrode 1201 and a drain electrode 1203, and gate electrodes 1204 are embedded in the active layer 1202, as illustrated in FIG. 2. The gate electrodes 1204 are electrically connected to a unit for applying gate voltage, and the source electrode 1201 and the drain electrode 1203 are electrically connected to a unit for controlling the voltage between the source and the drain.

In such an element structure, when voltage is applied between the source and the drain under the condition where gate voltage is not applied, current flows (on state). Then, by application of voltage to the gate electrode in that state, a depletion layer is formed in the periphery of the gate electrode 1204, and the current ceases flowing (off state). With such a mechanism, the element operates as a transistor.

Like a light-emitting element, a vertical transistor should contain a material that can achieve both a high carrier-transport property and high quality film for an active layer; a carbazole compound described in Embodiment 1 meets such a requirement and therefore can be suitably used.

### Embodiment 3

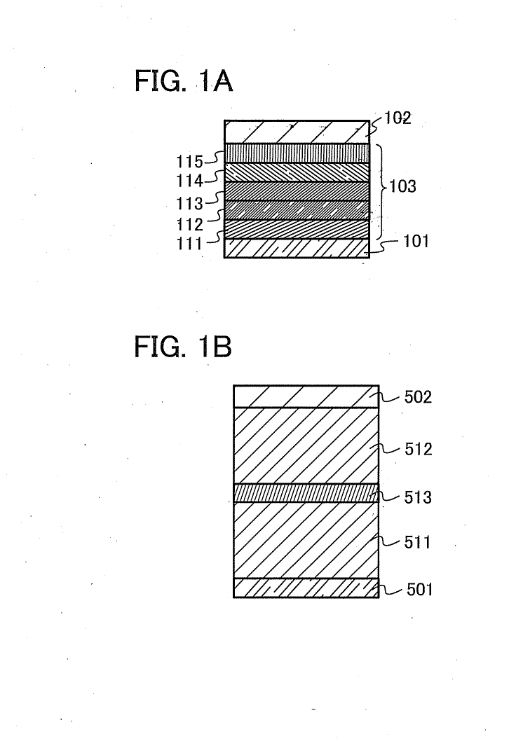

In this embodiment, one embodiment of a light-emitting element using any of the carbazole compounds described in Embodiment 1 will be described with reference to FIG. 1A.

A light-emitting element of this embodiment has a plurality of layers between a pair of electrodes. In this embodiment, the light-emitting element includes a first electrode 101, a second electrode 102, and a layer 103 containing an organic compound, which is provided between the first electrode 101 and the second electrode 102. Note that in this embodiment, the first electrode 101 functions as an anode and the second electrode 102 functions as a cathode. In other words, when voltage is applied between the first electrode 101 and the second electrode 102 so that the potential of the first electrode 101 is higher than that of the second electrode 102, light emission can be obtained.

For the first electrode 101, any of metals, alloys, electrically conductive compounds, and mixtures thereof which have a high work function (specifically, a work function of 4.0 eV or more) or the like is preferably used. Specific examples are indium oxide-tin oxide (ITO: indium tin oxide), indium oxide-tin oxide containing silicon or silicon oxide, indium oxide-zinc oxide, indium oxide containing tungsten oxide and zinc oxide (IWZO), and the like. Films of these electrically conductive metal oxides are usually formed by sputtering but may be formed by a sol-gel method or the like. For example, indium oxide-zinc oxide can be foinied by a sputtering method using a target in which zinc oxide is added to indium oxide at 1 wt % to 20 wt %. Moreover, indium oxide containing tungsten oxide and zinc oxide (IWZO) can be formed by a sputtering method using a target in which tungsten oxide is added to indium oxide at 0.5 wt % to 5 wt % and zinc oxide is added to indium oxide at 0.1 wt % to 1 wt %. Other examples are gold (Au), platinum (Pt), nickel (Ni), tungsten (W), chromium (Cr), molybdenum (Mo), iron (Fe), cobalt (Co), copper (Cu), palladium (Pd), a nitride of a metal material (such as titanium nitride), and the like. Graphene may also be used.

There is no particular limitation on the stacked structure of the layer 103 containing an organic compound. The layer 103 containing an organic compound can be formed by combining a layer containing a substance having a high electron-transport property, a layer containing a substance having a high hole-transport property, a layer containing a substance having a high electron-injection property, a layer containing a substance having a high hole-injection property, a layer containing a bipolar substance (a substance having a high electron-transport and hole-transport property), a layer having a carrier-blocking property, and the like as appropriate. In this embodiment, the layer 103 containing an organic compound has a structure in which a hole-injection layer 111, a hole-transport layer 112, a light-emitting layer 113, an electron-transport layer 114, and an electron-injection layer 115 are stacked in this order over the first electrode 101 functioning as an anode. Materials contained in the layers are specifically given below.

The hole-injection layer 111 is a layer containing a substance having a high hole-injection property. The hole-injection layer 111 can be formed using molybdenum oxide, vanadium oxide, ruthenium oxide, tungsten oxide, manganese oxide, or the like. The hole-injection layer 111 can also be formed using a phthalocyanine-based compound such as phthalocyanine (abbreviation: H₂Pc) or copper phthalocyanine (abbreviation: CuPc); an aromatic amine compound such as 4,4′-bis[N-(4-diphenylaminophenyl)-N-phenylamino]biphenyl (abbreviation: DPAB) or N,N′-bis[4-[bis(3-methylphenyl)amino]phenyl]-N,N′-diphenyl-[1,1′-biphenyl]-4,4′-diamine (abbreviation: DNTPD); a high molecule compound such as poly(ethylenedioxythiophene)/poly(styrenesulfonic acid) (PEDOT/PSS), or the like.

The hole-injection layer 111 can be formed using a composite material in which a substance exhibiting an electron-accepting property (hereinafter, simply referred to as “electron-accepting substance”) with respect to a substance having a high hole-transport property is contained in the substance having a high hole-transport property. In this specification, the composite material refers to not a material in which two materials are simply mixed but a material in the state where charge transfer between the materials can be caused by a mixture of a plurality of materials. This charge transfer includes charge transfer that can occur only when there is an auxiliary effect of an electric field.

Note that by using the material in which the electron-accepting substance is contained in the substance having a high hole-transport property, a material used for forming the electrode can be selected regardless of the work function of the electrode. In other words, besides a material having a high work function, a material having a low work function can be used for the first electrode 101. Examples of the electron-accepting substance are 7,7,8,8-tetracyano-2,3,5,6-tetrafluoroquinodimethane (abbreviation: F₄-TCNQ), chloranil, and the like. A transition metal oxide can also be used. In particular, an oxide of a metal belonging to any of Groups 4 to 8 of the periodic table can be suitably used. Specifically, vanadium oxide, niobium oxide, tantalum oxide, chromium oxide, molybdenum oxide, tungsten oxide, manganese oxide, and rhenium oxide are preferable because of their high electron-accepting properties. Among these, molybdenum oxide is especially preferable as the electron-accepting substance because it is stable in the air, has a low hygroscopic property, and is easily handled.

As the substance having a high hole-transport property used for the composite material, any of a variety of organic compounds such as an aromatic amine compound, a carbazole compound, an aromatic hydrocarbon, and a high molecular compound (such as an oligomer, a dendrimer, or a polymer) can be used. The organic compound used for the composite material is preferably an organic compound having a high hole-transport property. Specifically, a substance having a hole mobility of 1×10⁻⁶ cm²/Vs or higher is preferably used. Note that any other substance may be used as long as the substance has a hole-transport property higher than an electron-transport property. Specific examples of the organic compound that can be used as a substance having a high hole-transport property in the composite material are given below.

Examples of the aromatic amine compound are N,N′-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), N,N′-bis{4-[bis(3-methylphenylamino]phenyl}-N,N′-diphenyl-(1,1′-biphenyl)-4,4′-diamine (abbreviation: DNTPD), 1,3,5-tris[N-(4-diphenylaminophenyl)-N-phenylamino]benzene (abbreviation: DPA3B), and the like.

Specific examples of the carbazole compound that can be used for the composite material are 3-[N-(9-phenylcarbazol-3-yl)-N-phenylamino]-9-phenylcarbazole (abbreviation: PCzPCA1), 3,6-bis [N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), and the like.

Other examples of the carbazole compound that can be used for the composite material are 4,4′-di(N-carbazolyl)biphenyl (abbreviation: CBP), 1,3,5-tris[4-(N-carbazolyl)phenyl]benzene (abbreviation: TCPB), 9-[4-(10-phenyl-9-anthryl)phenyl]-9H-carbazole (abbreviation: CzPA), 1,4-bis[4-(N-carbazolyl)phenyl]-2,3,5,6-tetraphenylbenzene, and the like.

Examples of the aromatic hydrocarbon that can be used for the composite material are 2-tert-butyl-9,10-di(2-naphthy)anthracene (abbreviation: t-BuDNA), 2-tert-butyl-9,10-di(1-naphthylanthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3 ,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, 2,5,8,11-tetra(tert-butyl)perylene, and the like. Other examples are pentacene, coronene, and the like. As these aromatic hydrocarbons given here, it is preferable that an aromatic hydrocarbon having a hole mobility of 1×10⁻⁶ cm²/Vs or more and having 14 to 42 carbon atoms be used.

The aromatic hydrocarbon that can be used for the composite material may have a vinyl skeleton. Examples of the aromatic hydrocarbon having a vinyl group are 4,4′-bis(2,2-diphenylvinyl)biphenyl (abbreviation: DPVBi), 9,10-bis[4-(2,2-diphenylvinyl)phenyl]anthracene (abbreviation: DPVPA), and the like.

Other examples are high molecular compounds such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), and poly[N,N′-bis(4-butylphenyl)-N,N′-bis(phenyl)benzidine] (abbreviation: poly-TPD).

The hole-transport layer 112 is a layer containing a substance having a high hole-transport property. As the substance having a high hole-transport property, those given above as the substances having high hole-transport properties, which can be used for the above composite material, can also be used. Note that a detailed description is omitted to avoid repetition. Refer to the description of the composite material.

The light-emitting layer 113 is a layer containing a light-emitting substance. The light-emitting layer 113 may be formed using a film containing only a light-emitting substance or a film in which an emission center substance is dispersed in a host material.

There is no particular limitation on a material that can be used as the light-emitting substance or the emission center substance in the light-emitting layer 113, and light emitted from the material may be either fluorescence or phosphorescence. Examples of the above light-emitting substance or emission center substance are fluorescent substances and phosphorescent substances. Examples of the fluorescent substance are
N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA),
N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP),
4-(10-phenyl-9-anthryl)-4′-(9-phenyl-9H-carbazol-3-yl)triphenylamine (abbreviation: PCBAPA),
N,N″-(2-tert-butylanthracene-9,10-diyldi-4,1-phenylene)bis[N,N′,N′-triphenyl-1,4-phenylenediamine] (abbreviation: DPABPA),
N,9-diphenyl-N-[4-(9,10-diphenyl-2-anthryl)phenyl]-9H-carbazol-3-amine (abbreviation: 2PCAPPA),
N-[4-(9,10-diphenyl-2-anthryl)phenyl]-N,N′,N′-triphenyl-1,4-phenylenediamine (abbreviation: 2DPAPPA),
N,N,N′,N′,N″,N″,N′″,N′″-octaphenyldibenzo[g,p]chrysene-2,7,10,15-tetraamine (abbreviation: DBC1), coumarin 30,
N-(9,10-diphenyl-2-anthryl)-N,9-diphenyl-9H-carbazol-3-amine (abbreviation: 2PCAPA),
N-[9,10-bis(1,1′-biphenyl-2-yl)-2-anthryl]-N,9-diphenyl-9H-carbazol-3-amine (abbreviation: 2PCABPhA),
N-(9,10-diphenyl-2-anthryl)-N,N′,N′-triphenyl-1,4-phenylenediamine (abbreviation: 2DPAPA),
N-[9,10-bis(1,1′-biphenyl-2-yl)-2-anthryl]-N,N′,N′-triphenyl-1,4-phenylenediamine (abbreviation: 2DPABPhA),
9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene,
5,12-bis(1,1′-biphenyl-4-yl)-6,11-diphenyltetracene (abbreviation: BPT),
2-(2-{2-[4-(dimethylamino)phenyl]ethenyl}-6-methyl-4H-pyran-4-ylidene)propanedinitrile (abbreviation: DCM1),
2-{2-methyl-6-[2-(2,3,6,7-tetrahydro-1H,5H-benzo[ij]quinolizin-9-yDethenyl]-4H-pyran-4-ylidene}propanedinitrile (abbreviation: DCM2),
N,N,N′,N′-tetrakis(4-methylphenyl)tetracene-5,11-diamine (abbreviation: p-mPhTD),
7,14-diphenyl-N,N,N′,N′-tetrakis(4-methylphenyl)acenaphtho[1,2-a]fluoranthene-3,10-diamine (abbreviation: p-mPhAFD),
2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI),
2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB),
2-(2,6-bis{2-[4-(dimethylamino)phenyl]ethenyl}-4H-pyran-4-ylidene)propanedinitrile (abbreviation: BisDCM),
2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM),
N,N′-bis[4-(9-phenyl-9H-fluoren-9-yl)phenyl]-N,N′-diphenylpyrene-1,6-diamine (abbreviation: 1,6FLPAPm), and the like. Examples of the phosphorescent substance are bis[2-(3′,5′-bistrifluoromethylphenyl)pyridinato-N,C^{2′})iridium(III) picolinate (abbreviation: Ir(CF₃ppy)₂(pic)),
bis[2-(4′,6′-difluorophenyl)pyridinato-N,C^{2′})iridium(III) acetylacetonate (abbreviation: FIracac), tris(2-phenylpyridinato)iridium(III) (abbreviation: Ir(ppy)₃),
bis(2-phenylpyridinato)iridium(III) acetylacetonate (abbreviation: Ir(ppy)₂(acac)),
tris(acetylacetonato) (monophenanthroline)terbium(III) (abbreviation: Tb(acac)₃(Phen)),
bis(benzo[h]quinolinato)iridium(III) acetylacetonate (abbreviation: Ir(bzq)₂(acac)),
bis(2,4-diphenyl-1,3-oxazolato-N,C^{2′})iridium(III) acetylacetonate (abbreviation:
Ir(dpo)₂(acac)), bis[2-(4′-perfluorophenylphenyl)pyridinato]iridium(III) acetylacetonate (abbreviation: Ir(p-PF-ph)₂(acac)), bis(2-phenylbenzothiazolato-N,C^{2′})iridium(III) acetylacetonate (abbreviation: Ir(bt)₂(acac)),
bis[2-(2′-benzo[4,5-α]thienyl)pyridinato-N,C^{3′}]iridium(III) acetylacetonate (abbreviation: Ir(btp)₂(acac)), bis(1-phenylisoquinolinato-N,C^{2′})iridium(III) acetylacetonate (abbreviation: Ir(piq)₂(acac)), (acetylacetonato)bis[2,3-bis(4-fluorophenyl)quinoxalinato]iridium(III) (abbreviation: Ir(Fdpq)₂(acac)), (acetylacetonato)bis(2,3,5-triphenylpyrazinato)iridium(III) (abbreviation: Ir(tppr)₂(acac)), 2,3,7,8,12,13,17,18-octaethyl-21H,23H-porphine platinum(II) (abbreviation: PtOEP), tris(1,3-diphenyl-1,3-propanedionato)(monophenanthroline)europium(III) (abbreviation: Eu(DBM)₃(Phen)),
tris[1-(2-thenyl)-3,3,3-trifluoroacetonato](monophenanthroline)europium(III) (abbreviation: Eu(TTA)₃(Phen)), and the like. Note that a carbazole compound according to one embodiment of the present invention, a typical example of which is the carbazole compound represented by General Formula (G1) described in Embodiment 1, emits light in the blue to ultraviolet region, and therefore can also be used as an emission center substance.

The carbazole compound which is described in Embodiment 1 and represented by General Formula (G1) has a wide band gap and a high triplet excitation level (a large energy difference between a triplet excited state and a ground state); thus, the carbazole compound can be suitably used especially as a host material in which an emission center substance emitting blue fluorescence or an emission center substance emitting green to blue phosphorescence is dispersed. Needless to say, the carbazole compound can also be used as a host material in which an emission center substance emitting fluorescence with a wavelength longer than that of blue light or an emission center substance emitting phosphorescence with a wavelength longer than that of green light is dispersed. The carbazole compound may be used as a material contained in the carrier-transport layer adjacent to the light-emitting layer. Since the carbazole compound has a wide band gap or a high triplet excitation level, the energy of carriers that recombine in the host material can be efficiently transferred to an emission center substance even if the emission center substance is a substance that emits blue fluorescence or green to blue phosphorescence. Thus, a light-emitting element having high emission efficiency can be manufactured. Note that in the case where the carbazole compound which is described in Embodiment 1 and represented by General Formula (G1) is used as a host material, an emission center substance is preferably selected from, but not limited to, substances having a narrower band gap or a lower singlet excitation level or triplet excitation level than the carbazole compound.

Further, the carbazole compounds described in Embodiment 1 each have a high carrier-transport property. Thus, the use of the carbazole compound as a host material allows a light-emitting element having low driving voltage to be manufactured.

When the carbazole compound represented by the general formula (G1) is not used as the host material described above, any of the following substances can be used for the host material: metal complexes such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ); heterocyclic compounds such as 2-(4-biphenylyl)-5-(4-tert-butylphenyl)-1,3 ,4-oxadiazole (abbreviation: PBD), 1,3-bis[5-(p-tert-butylphenyl)-1,3,4-oxadiazol-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), bathophenanthroline (abbreviation: BPhen), bathocuproine (abbreviation: BCP), and 9-[4-(5-phenyl-1,3,4-oxadiazol-2-yl)phenyl]-9H-carbazole (abbreviation: CO11); and aromatic amine compounds such as 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), and 4,4′-bis[N-(spiro-9,9′-bifluoren-2-yl)-N-phenylamino]biphenyl (abbreviation: BSPB). Other examples are condensed polycyclic aromatic compounds such as anthracene derivatives, phenanthrene derivatives, pyrene derivatives, chrysene derivatives, and dibenzo[g,p]chrysene derivatives. Specific examples thereof are 9,10-diphenylanthracene (abbreviation: DPAnth),
N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), 4-(9H-carbazol-9-yl)-4′-(10-phenyl-9-anthryl)triphenylamine (abbreviation: YGAPA),
N,9-diphenyl-N-[4-(10-phenyl-9-anthryl)phenyl]-9H-carbazol-3-amine (abbreviation: PCAPA),
N,9-diphenyl-N-{4-[4-(10-phenyl-9-anthryl)phenyl]phenyl}-9H-carbazol-3-amine (abbreviation: PCAPBA),
N,9-diphenyl-N-(9,10-diphenyl-2-anthryl)-9H-carbazol-3-amine (abbreviation: 2PCAPA), 6,12-dimethoxy-5,11-diphenylchrysene,
N,N,N′,N′,N″,N″,N′″,N′″-octaphenyldibenzo[g,p]chrysene-2,7,10,15-tetramine (abbreviation: DBC1), 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA),
2-tert-butyl-9,10-di(2-naphthyDanthracene (abbreviation: t-BuDNA), 9,9′-bianthryl (abbreviation: BANT), 9,9′-(stilbene-3,3′-diyldiphenanthrene (abbreviation: DPNS),
9,9′-(stilbene-4,4′-diyl)diphenanthrene (abbreviation: DPNS2),
3,3′,3″-(benzene-1,3,5-triyltripyrene (abbreviation: TPB3), and the like. Other than these, known materials can be given.

The light-emitting layer 113 may be a stack of two or more layers. For example, in the case where the light-emitting layer 113 is fainted by stacking a first light-emitting layer and a second light-emitting layer in this order over the hole-transport layer, a structure can be employed in which the first light-emitting layer serves as a layer having a hole-transport property and the second light-emitting layer serves as a layer having an electron-transport property.

In the case where the light-emitting layer having the above-described structure is formed using a plurality of materials, the light-emitting layer can be founed using co-evaporation by a vacuum evaporation method; or an inkjet method, a spin coating method, a dip coating method, or the like with a solution of the materials.

The electron-transport layer 114 is a layer containing a substance having a high electron-transport property. For example, the electron-transport layer 114 is formed using a metal complex having a quinoline skeleton or a benzoquinoline skeleton, such as tris(8-quinolinolato)aluminum (abbreviation: Alq),
tris(4-methyl-8-quinolinolato)aluminum (abbreviation: Almq₃),
bis(10-hydroxybenzo[h]-quinolinato)beryllium (abbreviation: BeBq₂), or
bis(2-methyl-8-quinolinolato)(4-phenylphenolato)aluminum (abbreviation: BAlq), or the like. A metal complex having an oxazole-based or thiazole-based ligand, such as
bis[2-(2-hydroxyphenyl)benzoxazolato]zinc (abbreviation: Zn(BOX)₂) or
bis[2-(2-hydroxyphenyl)benzothiazolato]zinc (abbreviation: Zn(BTZ)₂), or the like can also be used. Other than the metal complexes,
2-(4-biphenylyl)-5-(4-tert-butylphenyl)-1,3,4-oxadiazole (abbreviation: PBD),
1,3-bis[5-(p-tert-butylphenyl)-1,3,4-oxadiazol-2-yl]benzene (abbreviation: OXD-7),
3-(4-biphenylyl)-4-phenyl-5-(4-tert-butylphenyl)-1,2,4-triazole (abbreviation: TAZ), bathophenanthroline (abbreviation: BPhen), bathocuproine (abbreviation: BCP), or the like can also be used. The substances given here are mainly ones having an electron mobility of 10⁻⁶ cm²/Vs or higher. Note that any substance other than the above substances may be used for the electron-transport layer as long as the substance has an electron-transport property higher than a hole-transport property.

The carbazole compounds described in Embodiment 1 may also be used as a material contained in the electron-transport layer 114. The carbazole compounds described in Embodiment 1 each have a wide band gap and a high T₁ level and thus can effectively prevent transfer of excitation energy in the light-emitting layer to the electron-transport layer 114 to suppress a reduction in emission efficiency due to the excitation energy transfer, and allow a light-emitting element having high emission efficiency to be manufactured. Moreover, the carbazole compounds described in Embodiment 1 each have a high carrier-transport property; thus, a light-emitting element having low driving voltage can be provided.

The electron-transport layer is not limited to a single layer and may be a stack of two or more layers containing any of the above substances.

A layer for controlling transport of electron carriers may be provided between the electron-transport layer and, the light-emitting layer. This is a layer fanned by addition of a small amount of a substance having a high electron-trapping property to a material having a high electron-transport property as described above, and the layer is capable of adjusting carrier balance by suppressing transport of electron carriers. Such a structure is very effective in preventing a problem (such as a reduction in element lifetime) caused when electrons pass through the light-emitting layer.

In addition, an electron-injection layer 115 may be provided in contact with the second electrode 102, between the electron-transport layer 114 and the second electrode 102. For the electron-injection layer 115, an alkali metal, an alkaline earth metal, or a compound thereof such as lithium, calcium, lithium fluoride (LiF), cesium fluoride (CsF), or calcium fluoride (CaF₂) can be used. A composite material of a substance having an electron-transport property and a substance exhibiting an electron-donating property (hereinafter, simply referred to as “electron-donating substance”) with respect to the substance having an electron-transport property can also be used. Examples of the electron-donating substance are alkali metals, alkaline earth metals, and compounds thereof. For example, as the composite material, a composite material in which magnesium (Mg) is contained in Alq, or the like can be used. Note that a layer which is formed using a substance having an electron-transport property and contains an alkali metal or an alkaline earth metal is preferably used for the electron-injection layer 115, in which case electrons are efficiently injected from the second electrode 102. With such a structure, a conductive material as well as a substance having a low work function can be used for the cathode.

For the second electrode 102, any of metals, alloys, electrically conductive compounds, and mixtures thereof which have a low work function (specifically, a work function of 3.8 eV or less), and the like can be used. Specific examples of such a cathode material include elements belonging to Groups 1 and 2 in the periodic table, i.e., alkali metals such as lithium (Li) and cesium (Cs), and alkaline earth metals such as calcium (Ca) and strontium (Sr), magnesium (Mg), alloys containing any of the metals (e.g., MgAg or AlLi), rare earth metals such as europium (Eu) and ytterbium (Yb), alloys containing any of the metals, and the like. However, when the electron-injection layer is provided between the second electrode 102 and the electron-transport layer, for the second electrode 102, any of a variety of conductive materials such as Al, Ag, ITO, or indium oxide-tin oxide containing silicon or silicon oxide can be used regardless of the work function. Films of these conductive materials can be formed by a sputtering method, an ink jet method, a spin coating method, or the like.

Further, any of a variety of methods can be employed for forming the layer 103 containing an organic compound regardless of a dry process or a wet process. For example, a vacuum evaporation method, an ink-jet method, a spin coating method or the like may be employed. A different formation method may be employed for each electrode or each layer.

The electrode may be formed by a wet process using a sol-gel method, or by a wet process using paste of a metal material. Alternatively, the electrode may be formed by a dry process such as a sputtering method or a vacuum evaporation method.

In the light-emitting element having the above-described structure, current flows due to a potential difference between the first electrode 101 and the second electrode 102, and holes and electrons recombine in the light-emitting layer 113 which contains a substance having a high light-emitting property, so that light is emitted. In other words, a light-emitting region is farmed in the light-emitting layer 113.

Light is extracted out through one or both of the first electrode 101 and the second electrode 102. Therefore, one or both of the first electrode 101 and the second electrode 102 are light-transmitting electrodes. In the case where only the first electrode 101 is a light-transmitting electrode, light is extracted from the substrate side through the first electrode 101. In contrast, when only the second electrode 102 is a light-transmitting electrode, light is extracted from the side opposite to the substrate side through the second electrode 102. In the case where both the first electrode 101 and the second electrode 102 are light-transmitting electrodes, light is extracted from both the substrate side and the side opposite to the substrate side through the first electrode 101 and the second electrode 102.

The structure of the layers provided between the first electrode 101 and the second electrode 102 is not limited to the above-described structure. However, it is preferable that a light-emitting region where holes and electrons recombine be positioned away from the first electrode 101 and the second electrode 102 so as to prevent quenching due to the proximity of the light-emitting region and a metal used for an electrode or a carrier-injection layer.

Further, in order that transfer of energy from an exciton generated in the light-emitting layer can be suppressed, it is preferable that the hole-transport layer and the electron-transport layer which are in direct contact with the light-emitting layer, particularly a carrier-transport layer in contact with a side closer to the light-emitting region in the light-emitting layer 113 be formed using a substance having a larger energy gap than the light-emitting substance of the light-emitting layer or the emission center substance contained in the light-emitting layer.

Since the light-emitting element of this embodiment is formed using any of the carbazole compounds described in Embodiment 1, which has a wide energy gap, as a host material and/or for the electron-transport layer, efficient light emission can be obtained even if an emission center substance has a wide energy gap and emits blue fluorescence or green to blue phosphorescence, and the light-emitting element can have high emission efficiency. Thus, a light-emitting element with lower power consumption can be provided. In addition, light emission from a host material or a material contained in a carrier-transport layer is unlikely to occur; thus, a light-emitting element that provides light emission with high color purity can be provided. Further, the carbazole compounds described in Embodiment 1 each have a high carrier-transport property; thus, a light-emitting element having low driving voltage can be provided.

Such a light-emitting element may be manufactured using a substrate made of glass, plastic, or the like as a support. A plurality of such light emitting elements are formed over one substrate, thereby forming a passive matrix light emitting device. Alternatively, a transistor may be formed over a substrate made of glass, plastic, or the like, and the light-emitting element may be manufactured over an electrode electrically connected to the transistor. In this manner, an active matrix light-emitting device in which the driving of the light-emitting element is controlled by the transistor can be manufactured. Note that a structure of the transistor is not particularly limited. Either a staggered TFT or an inverted staggered TFT may be employed. In addition, the crystallinity of a semiconductor used for the TFT is not particularly limited. In addition, a driver circuit formed in a TFT substrate may be formed with n-type TFTs and p-type TFTs, or with either n-type TFTs or p-type TFTs. The semiconductor layer for forming the TFTs may be formed using any material as long as the material exhibits semiconductor characteristics; for example, an element belonging to Group 14 of the periodic table such as silicon (Si) and germanium (Ge), a compound such as gallium arsenide and indium phosphide, an oxide such as zinc oxide and tin oxide, and the like can be given. For the oxide exhibiting semiconductor characteristics (oxide semiconductor), composite oxide of an element selected from indium, gallium, aluminum, zinc, and tin can be used. Examples thereof are zinc oxide (ZnO), indium oxide containing zinc oxide (indium zinc oxide), and oxide containing indium oxide, gallium oxide, and zinc oxide (IGZO: indium gallium zinc oxide). An organic semiconductor layer may also be used. The semiconductor layer may have either a crystalline structure or an amorphous structure. Specific examples of the crystalline semiconductor layer are a single crystal semiconductor, a polycrystalline semiconductor, and a microcrystalline semiconductor.

### Embodiment 4

In this embodiment, an embodiment of a light-emitting element with a structure in which a plurality of light-emitting units are stacked (hereinafter, also referred to as “stacked-type element”) will be described with reference to FIG. 1B. This light-emitting element is a light-emitting element including a plurality of light-emitting units between a first electrode and a second electrode. One light-emitting unit has the same structure as the layer 103 containing an organic compound which is described in Embodiment 3. In other words, the light-emitting element described in Embodiment 3 includes one light-emitting unit while the light-emitting element in this embodiment includes a plurality of light-emitting units.

In FIG. 1B, a first light-emitting unit 511 and a second light-emitting unit 512 are stacked between a first electrode 501 and a second electrode 502, and a charge-generation layer 513 is provided between the first light-emitting unit 511 and the second light-emitting unit 512. The first electrode 501 and the second electrode 502 correspond, respectively, to the first electrode 101 and the second electrode 102 in Embodiment 3, and materials described in Embodiment 3 can be used. Further, the first light-emitting unit 511 and the second light-emitting unit 512 may have the same structure or different structures.

The charge-generation layer 513 contains a composite material of an organic compound and a metal oxide. This composite material of an organic compound and a metal oxide is the composite material which can be used for the hole-injection layer as described in Embodiment 3, and contains an organic compound and a metal oxide such as vanadium oxide, molybdenum oxide, or tungsten oxide. As the organic compound, a variety of compounds such as an aromatic amine compound, a carbazole compound, an aromatic hydrocarbon, and a high molecular compound (such as an oligomer, a dendrimer, or a polymer) can be used. An organic compound having a hole mobility of 1×10⁻⁶ cm²/Vs or higher is preferably used as a hole-transport organic compound. However, any other substance may be used as long as the substance has a hole-transport property higher than an electron-transport property. The composite material of an organic compound and a metal oxide has a high carrier-injection property and a high carrier-transport property; thus, low-voltage driving and low-current driving can be achieved.

The charge-generation layer 513 may have a stacked-layer structure of a layer containing the composite material of an organic compound and a metal oxide and a layer containing another material. For example, a layer containing the composite material of an organic compound and a metal oxide may be combined with a layer containing a compound of a substance selected from electron-donating substances and a compound having a high electron-transport property. Moreover, the charge-generation layer 513 may be formed by combining a layer containing the composite material of an organic compound and a metal oxide with a transparent conductive film.

The charge-generation layer 513 provided between the first light-emitting unit 511 and the second light-emitting unit 512 may have any structure as far as electrons can be injected to a light-emitting unit on one side and holes can be injected to a light-emitting unit on the other side when a voltage is applied between the first electrode 501 and the second electrode 502. For example, in FIG. 1B, any layer can be used as the charge generation layer 513 as far as the layer injects electrons into the first light-emitting unit 511 and holes into the second light-emitting unit 512 when a voltage is applied such that the voltage of the first electrode is higher than that of the second electrode.

Although the light-emitting element having two light-emitting units is described in this embodiment, the present invention can be similarly applied to a light-emitting element in which three or more light-emitting units are stacked. With a plurality of light-emitting units partitioned by the charge-generation layer between a pair of electrodes as in the light-emitting element according to this embodiment, it is possible to provide a light-emitting element which can emit light with high luminance with the current density kept low and has a long lifetime. Moreover, a light-emitting device having low driving voltage and lower power consumption can be achieved.

Further, when emission colors of the light-emitting units are made different, light emission having a desired color can be obtained from the light-emitting element as a whole. For example, in the light-emitting element having two light-emitting units, when an emission color of the first light-emitting unit and an emission color of the second light-emitting unit are made to be complementary colors, it is possible to obtain a light-emitting element from which white light is emitted from the whole light-emitting element. Note that “complementary colors” refer to colors that can produce an achromatic color when mixed. In other words, when lights obtained from substances which emit complementary colors are mixed, white emission can be obtained. This can be applied to a light-emitting element having three light-emitting units. For example, when the first light-emitting unit emits red light, the second light-emitting unit emits green light, and the third light-emitting unit emits blue light, white light can be emitted from the whole light-emitting element.

The light-emitting element of this embodiment includes any of the carbazole compounds described in Embodiment 1 and thus can have high emission efficiency. In addition, the light-emitting element can have low driving voltage. In addition, the light-emitting unit containing the carbazole compound can provide light with high color purity, which originates from the emission center substance; therefore, it is easy to adjust the color of light emitted from the light-emitting element as a whole.

Note that this embodiment can be combined with any of the other embodiments as appropriate.

### Embodiment 5

In this embodiment, a light-emitting device including a light-emitting element including any of the carbazole compounds described in Embodiment 1 will be described.

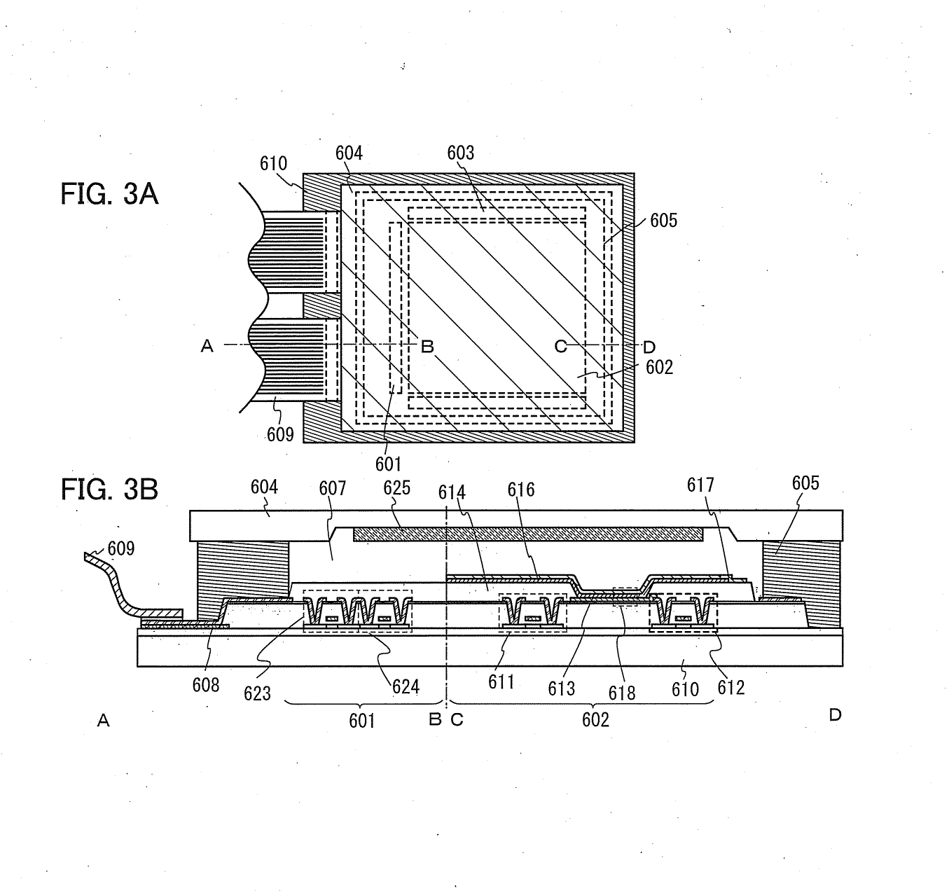

In this embodiment, an example of the light-emitting device manufactured using a light-emitting element including any of the carbazole compounds described in Embodiment 1 will be described with reference to FIGS. 3A and 3B. Note that FIG. 3A is a top view illustrating the light-emitting device and FIG. 3B is a cross-sectional view of FIG. 3A taken along lines A-A′ and B-B′. The light-emitting device includes a driver circuit portion (source driver circuit) 601, a pixel portion 602, and a driver circuit portion (gate driver circuit) 603 which are illustrated with dotted lines. These units control light emission of the light-emitting element. Moreover, a reference numeral 604 denotes a sealing substrate; 605, a sealing material; and 607, a space surrounded by the sealing material 605.

Reference numeral 608 denotes a wiring for transmitting signals to be input into the source driver circuit 601 and the gate driver circuit 603 and receiving signals such as a video signal, a clock signal, a start signal, and a reset signal from a flexible printed circuit (FPC) 609 serving as an external input teiminal. Although only the FPC is illustrated here, a printed wiring board (PWB) may be attached to the FPC. The light-emitting device in the present specification includes, in its category, not only the light-emitting device itself but also the light-emitting device provided with the FPC or the PWB.

Next, a cross-sectional structure is described with reference to FIG. 3B. The driver circuit portion and the pixel portion are formed over an element substrate 610; the source driver circuit 601, which is a driver circuit portion, and one of the pixels in the pixel portion 602 are illustrated here.

In the source driver circuit 601, a CMOS circuit in which an n-channel TFT 623 and a p-channel TFT 624 are combined is formed. Such a driver circuit may be formed using a variety of circuits such as a CMOS circuit, a PMOS circuit, or an NMOS circuit. Although a driver-integrated type in which the driver circuit is formed over the substrate is described in this embodiment, the present invention is not limited to this type and the driver circuit can be formed outside the substrate.

The pixel portion 602 includes a plurality of pixels each including a switching TFT 611, a current controlling TFT 612, and a first electrode 613 electrically connected to a drain of the current controlling TFT 612. Note that an insulator 614 is formed to cover an edge portion of the first electrode 613. In this embodiment, the insulator 614 is formed using a positive photosensitive acrylic film

In order to improve the coverage, the insulator 614 is formed to have a curved surface with curvature at its upper or lower end portion. For example, in the case of using positive photosensitive acrylic for the insulator 614, only the upper end portion of the insulator 614 preferably has a curved surface with a radius of curvature of 0.2 μm to 3 μm. Moreover, either a negative photosensitive resin or a positive photosensitive resin can be used as the insulator 614.

A layer 616 containing an organic compound and a second electrode 617 are formed over the first electrode 613. As a material used for the first electrode 613 which functions as an anode, a material having a high work function is preferably used. For example, a single-layer film of an ITO film, an indium tin oxide film containing silicon, an indium oxide film containing zinc oxide at 2 wt % to 20 wt %, a titanium nitride film, a chromium film, a tungsten film, a Zn film, a Pt film, or the like, a stack of a titanium nitride film and a film containing aluminum as its main component, a stack of three layers of a titanium nitride film, a film containing aluminum as its main component, and a titanium nitride film, or the like can be used. The stacked structure achieves low wiring resistance, a favorable ohmic contact, and a function as an anode.

In addition, the layer 616 containing an organic compound is formed by any of a variety of methods such as an evaporation method using an evaporation mask, an inkjet method, and a spin coating method. The layer 616 containing an organic compound contains any of the carbazole compounds described in Embodiment 1. Further, the layer 616 containing an organic compound may be formed using another material such as a low molecular compound or a high molecular compound (e.g., an oligomer or a dendrimer).

As a material used for the second electrode 617, which is formed over the layer 616 containing an organic compound and functions as a cathode, a material having a low work function (e.g., Al, Mg, Li, Ca, or an alloy or compound thereof, such as MgAg, MgIn, or AILi) is preferably used. In the case where light generated in the layer 616 containing an organic compound passes through the second electrode 617, a stack of a thin metal film and a transparent conductive film (e.g., ITO, indium oxide containing zinc oxide at 2 wt % to 20 wt %, indium tin oxide containing silicon, or zinc oxide (ZnO)) is preferably used for the second electrode 617.

Note that the light-emitting element is formed with the first electrode 613, the layer 616 containing an organic compound, and the second electrode 617. The light-emitting element has the structure described in Embodiment 3 or 4. In the light-emitting device of this embodiment, the pixel portion, which includes a plurality of light-emitting elements, may include both the light-emitting element with the structure described in Embodiment 3 or 4 and a light-emitting element with a structure other than those.

Further, the sealing substrate 604 is attached to the element substrate 610 with the sealing material 605, so that a light-emitting element 618 is provided in the space 607 surrounded by the element substrate 610, the sealing substrate 604, and the sealing material 605. The space 607 may be filled with filler, and may be filled with an inert gas (e.g., nitrogen or argon), or the sealing material 605.

An epoxy-based resin is preferably used for the sealing material 605. It is preferable that such a material do not transmit moisture or oxygen as much as possible. As the sealing substrate 604, a glass substrate, a quartz substrate, or a plastic substrate formed of fiberglass reinforced plastic (FRP), polyvinyl fluoride (PVF), polyester, acrylic, or the like can be used.

As described above, the light-emitting device manufactured using the light-emitting element including any of the carbazole compounds described in Embodiment 1 can be obtained.

The light-emitting device of this embodiment is manufactured using the light-emitting element including any of the carbazole compounds described in Embodiment 1 and thus can have good characteristics. Specifically, since the carbazole compounds described in Embodiment 1 each have a wide energy gap and a high triplet excitation level and can prevent energy transfer from a light-emitting substance, a light-emitting element having high emission efficiency can be provided; thus, a light-emitting device having reduced power consumption can be provided. In addition, a light-emitting element having low driving voltage can be provided; thus, a light-emitting device having low driving voltage can be provided.

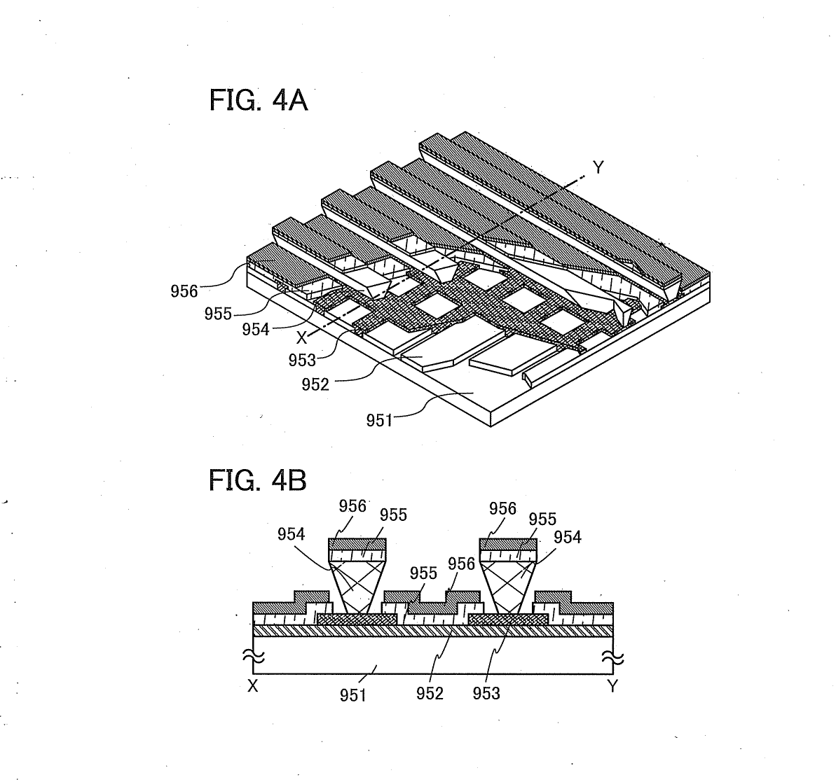

An active matrix light-emitting device is described above, whereas a passive matrix light-emitting device is described below. FIGS. 4A and 4B illustrate a passive matrix light-emitting device manufactured according to the present invention. FIG. 4A is a perspective view of the light-emitting device, and FIG. 4B is a cross-sectional view of FIG. 4A taken along line X-Y. In FIGS. 4A and 4B, over a substrate 951, a layer 955 containing an organic compound is provided between an electrode 952 and an electrode 956. An edge portion of the electrode 952 is covered with an insulating layer 953. A partition layer 954 is provided over the insulating layer 953. The sidewalls of the partition layer 954 slope so that the distance between one sidewall and the other sidewall gradually decreases toward the surface of the substrate. In other words, a cross section taken along the direction of the short side of the partition layer 954 is trapezoidal, and the base (a side which is in the same direction as a plane direction of the insulating layer 953 and in contact with the insulating layer 953) is shorter than the upper side (a side which is in the same direction as the plane direction of the insulating layer 953 and not in contact with the insulating layer 953). By providing the partition layer 954 in such a manner, a defect of the light-emitting element due to static electricity or the like can be prevented. The passive matrix light-emitting device can also be driven while power consumption is kept low, by including the light-emitting element described in Embodiment 3 or 4 which includes any of the carbazole compounds described in Embodiment 1. and is capable of operating at low voltage. In addition, the light-emitting device can be driven with low driving voltage by including the light-emitting element described in Embodiment 3 or 4 which includes any of the carbazole compounds described in Embodiment 1 and therefore has high emission efficiency.

Since many minute light-emitting elements arranged in a matrix in the light-emitting device described above can each be controlled, the light-emitting device can be suitably used as a display device for displaying images.

### Embodiment 6

In this embodiment, electronic devices each including the light-emitting element described in Embodiment 3 or 4 will be described. The light-emitting element described in Embodiment 3 or 4 includes any of the carbazole compounds described in Embodiment 1 and thus has reduced power consumption; as a result, the electronic devices described in this embodiment can each include a display portion having reduced power consumption. In addition, the electronic devices can have low driving voltage since the light-emitting element described in Embodiment 3 or 4 has low driving voltage.

Examples of the electronic device to which the above light-emitting element is applied include television devices (also referred to as TV or television receivers), monitors for computers and the like, cameras such as digital cameras and digital video cameras, digital photo frames, cellular phones (also referred to as mobile phones or mobile phone devices), portable game machines, portable information terminals, audio playback devices, large game machines such as pachinko machines, and the like. Specific examples of these electronic devices are given below.

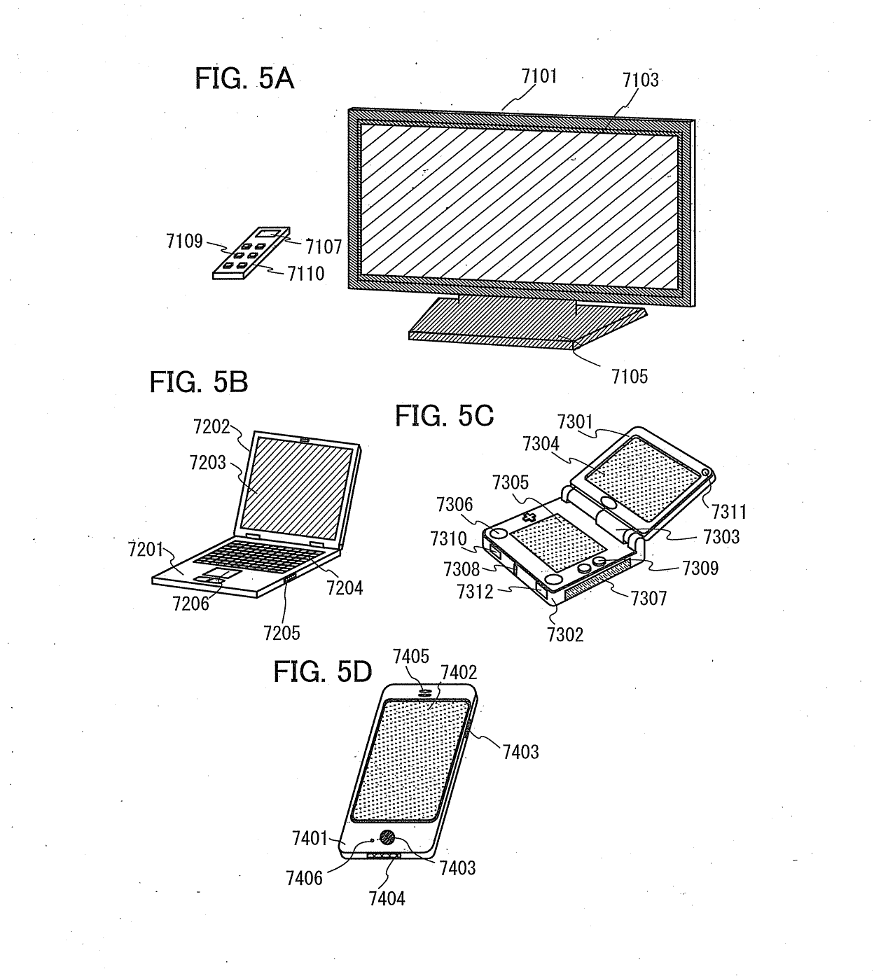

FIG. 5A illustrates an example of a television device. In the television device, a display portion 7103 is incorporated in a housing 7101. In addition, here, the housing 7101 is supported by a stand 7105. The display portion 7103 enables display of images and includes light-emitting elements which are the same as the light-emitting element described in Embodiment 3 or 4 and arranged in a matrix. The light-emitting elements each include any of the carbazole compounds described in Embodiment 1 and thus can have high emission efficiency and low driving voltage. Therefore, the television device including the display portion 7103 which is formed using the light-emitting elements can have reduced power consumption and low driving voltage.

The television device can be operated with an operation switch of the housing 7101 or a separate remote controller 7110. With operation keys 7109 of the remote controller 7110, channels and volume can be controlled and images displayed on the display portion 7103 can be controlled. Furthermore, the remote controller 7110 may be provided with a display portion 7107 for displaying data output from the remote controller 7110.

Note that the television device is provided with a receiver, a modem, and the like. With the use of the receiver, general television broadcasting can be received. Moreover, when the television set is connected to a communication network with or without wires via the modem, one-way (from a sender to a receiver) or two-way (between a sender and a receiver or between receivers) information communication can be performed.

FIG. 5B illustrates a computer, which includes a main body 7201, a housing 7202, a display portion 7203, a keyboard 7204, an external connection port 7205, a pointing device 7206, and the like. Note that this computer is manufactured by using light-emitting elements arranged in a matrix in the display portion 7203, which are the same as that described in Embodiment 3 or 4. The light-emitting elements each include any of the carbazole compounds described in Embodiment 1 and thus can have high emission efficiency and low driving voltage. Therefore, the computer including the display portion 7203 which is formed using the light-emitting elements can have reduced power consumption and low driving voltage.

FIG. 5C illustrates a portable game machine having two housings, a housing 7301 and a housing 7302, which are connected with a joint portion 7303 so that the portable game machine can be opened or folded. A display portion 7304 including light-emitting elements which are the same as that described in Embodiment 3 or 4 and arranged in a matrix is incorporated in the housing 7301, and a display portion 7305 is incorporated in the housing 7302. In addition, the portable game machine illustrated in FIG. 5C includes a speaker portion 7306, a recording medium insertion portion 7307, an LED lamp 7308, input means (an operation key 7309, a connection terminal 7310, a sensor 7311 (a sensor having a function to measure force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), and a microphone 7312), and the like. Needless to say, the structure of the portable game machine is not limited to the above as far as the display portion including light-emitting elements which are the same as that described in Embodiment 3 or 4 and arranged in a matrix is used as at least either the display portion 7304 or the display portion 7305, or both, and the structure can include other accessories as appropriate. The portable game machine illustrated in FIG. 5C has a function to read out a program or data stored in a storage medium to display it on the display portion, and a function to share information with another portable game machine by wireless communication. The portable game machine illustrated in FIG. 5C can have a variety of functions without limitation to the above. Since the light-emitting elements used in the display portion 7304 have high emission efficiency by including any of the carbazole compounds described in Embodiment 1, the portable game machine including the above-described display portion 7304 can be a portable game machine having reduced power consumption. Since the light-emitting elements used in the display portion 7304 each have low driving voltage by including any of the carbazole compounds described in Embodiment 1, the portable game machine can also be a portable game machine having low driving voltage.

FIG. 5D illustrates an example of a mobile phone. The mobile phone is provided with a display portion 7402 incorporated in a housing 7401, operation buttons 7403, an external connection port 7404, a speaker 7405, a microphone 7406, and the like. Note that the mobile phone has the display portion 7402 including light-emitting elements which are the same as that described in Embodiment 3 or 4 and arranged in a matrix. The light-emitting elements each include any of the carbazole compounds described in Embodiment 1 and thus can have high emission efficiency and low driving voltage. Therefore, the mobile phone including the display portion 7402 which is formed using the light-emitting elements can have reduced power consumption and low driving voltage.

When the display portion 7402 of the mobile phone illustrated in FIG. 5D is touched with a finger or the like, data can be input into the mobile phone. In this case, operations such as making a call and creating an e-mail can be performed by touching the display portion 7402 with a finger or the like.

There are mainly three screen modes of the display portion 7402. The first mode is a display mode mainly for displaying an image. The second mode is an input mode mainly for inputting information such as characters. The third mode is a display-and-input mode in which two modes of the display mode and the input mode are combined.

For example, in the case of making a call or creating an e-mail, a character input mode mainly for inputting characters is selected for the display portion 7402 so that characters displayed on a screen can be input. In this case, it is preferable to display a keyboard or number buttons on almost the entire screen of the display portion 7402.

When a detection device including a sensor for detecting inclination, such as a gyroscope or an acceleration sensor, is provided inside the cellular phone, display on the screen of the display portion 7402 can be automatically changed by determining the orientation of the cellular phone (whether the cellular phone is placed horizontally or vertically for a landscape mode or a portrait mode).

The screen modes are switched by touch on the display portion 7402 or operation with the operation buttons 7403 of the housing 7401. The screen modes can be switched depending on the kind of images displayed on the display portion 7402. For example, when a signal of an image displayed on the display portion is a signal of moving image data, the screen mode is switched to the display mode. When the signal is a signal of text data, the screen mode is switched to the input mode.

Moreover, in the input mode, when input by touching the display portion 7402 is not performed for a certain period while a signal detected by an optical sensor in the display portion 7402 is detected, the screen mode may be controlled so as to be switched from the input mode to the display mode.

The display portion 7402 may function as an image sensor. For example, an image of a palm print, a fingerprint, or the like is taken by touch on the display portion 7402 with the palm or the finger, whereby personal authentication can be performed. Further, by providing a backlight or a sensing light source which emits a near-infrared light in the display portion, an image of a fmger vein, a palm vein, or the like can be taken.

Note that the structure described in this embodiment can be combined with any of the structures described in Embodiments 1 to 5 as appropriate.

As described above, the application range of the light-emitting device having the light-emitting element described in Embodiment 3 or 4 which includes a carbazole compound described in Embodiment 1 is wide so that this light-emitting device can be applied to electronic devices in a variety of fields. By using any of the carbazole compounds described in Embodiment 1, an electronic device having reduced power consumption and low driving voltage can be obtained.

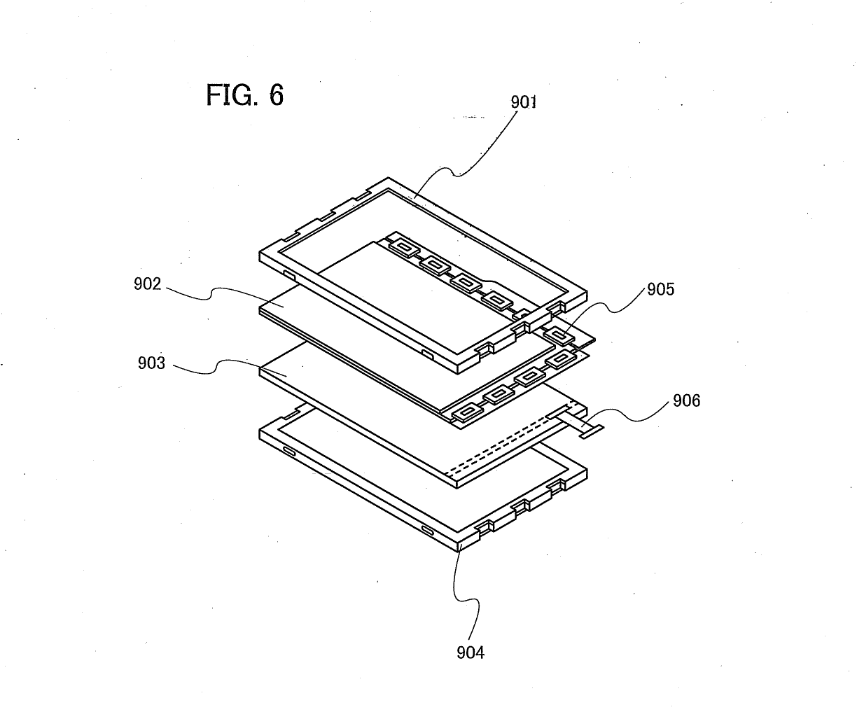

The light-emitting element including any of the carbazole compounds described in Embodiment 1 can also be used for a light source device. One mode of application of the light-emitting element including any of the carbazole compounds described in Embodiment 1 to a light source device is described with reference to FIG. 6. Note that the light source device includes a light-emitting element including any of the carbazole compounds described in Embodiment 1 as a light irradiation unit and at least includes an input-output terminal portion which supplies current to the light-emitting element. Further, the light-emitting element is preferably shielded from the outside atmosphere by sealing.

FIG. 6 illustrates an example of a liquid crystal display device using the light-emitting elements including any of the carbazole compounds described in Embodiment 1 for a backlight. The liquid crystal display device illustrated in FIG. 6 includes a housing 901, a liquid crystal layer 902, a backlight 903, and a housing 904. The liquid crystal layer 902 is connected to a driver IC 905. The light-emitting element including any of the carbazole compounds described in Embodiment 1 is used in the backlight 903, to which current is supplied through a terminal 906.

The light-emitting element including any of the carbazole compounds described in Embodiment 1 is used for the backlight of the liquid crystal display device; thus, the backlight can have reduced power consumption. In addition, the use of the light-emitting element including any of the carbazole compounds described in Embodiment 1 enables manufacture of a planar-emission lighting device and further a larger-area planar-emission lighting device; therefore, the backlight can be a larger-area backlight, and the liquid crystal display device can also be a larger-area device. Furthermore, the backlight using the light-emitting element including any of the carbazole compounds described in Embodiment 1 can be thinner than a conventional one; accordingly, the display device can also be thinner.

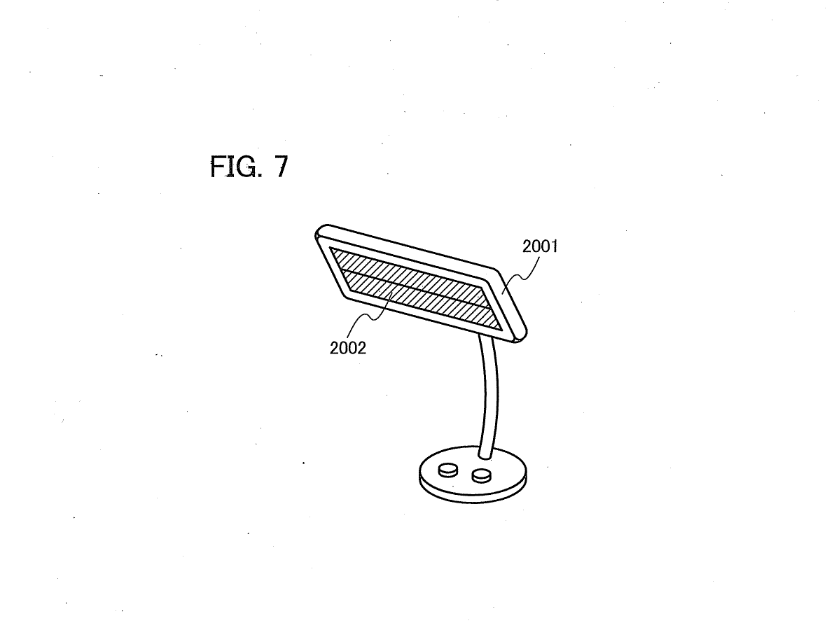

FIG. 7 illustrates an example in which the light-emitting element including any of the carbazole compounds described in Embodiment 1 is used for a table lamp which is a lighting device. The table lamp illustrated in FIG. 7 includes a housing 2001 and a light source 2002, and the light-emitting element including any of the carbazole compounds described in Embodiment 1 is used for the light source 2002.

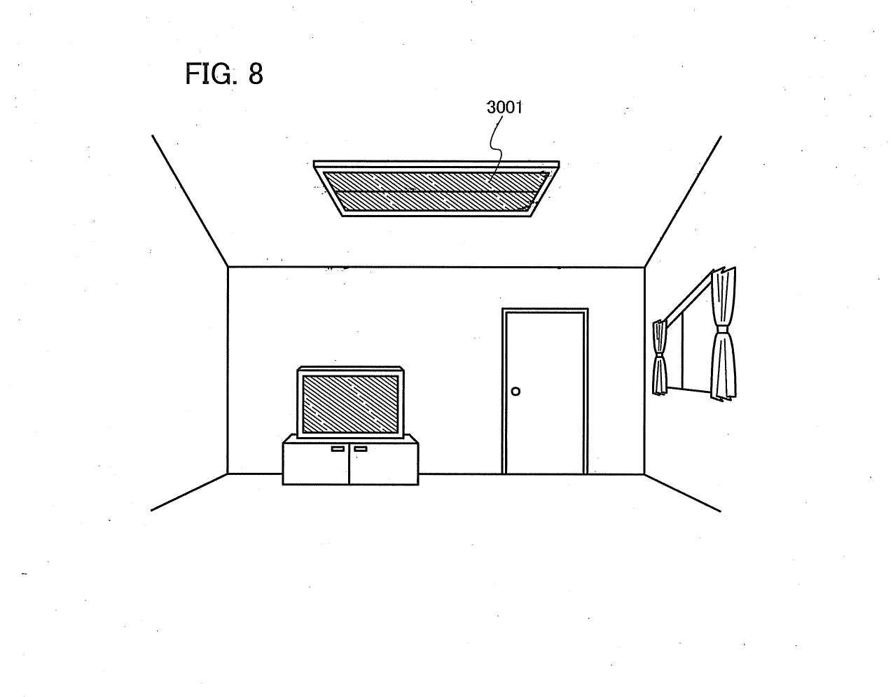

FIG. 8 illustrates an example in which the light-emitting element including any of the carbazole compounds described in Embodiment 1 is used for an indoor lighting device 3001. Since the light-emitting element including any of the carbazole compounds described in Embodiment 1 has reduced power consumption, a lighting device that has reduced power consumption can be obtained. Further, since the light-emitting element including any of the carbazole compounds described in Embodiment 1 can have a large area, the light-emitting element can be used for a large-area lighting device. Furthermore, since the light-emitting element including any of the carbazole compounds described in Embodiment 1 is thin, a lighting device having a reduced thickness can be manufactured.

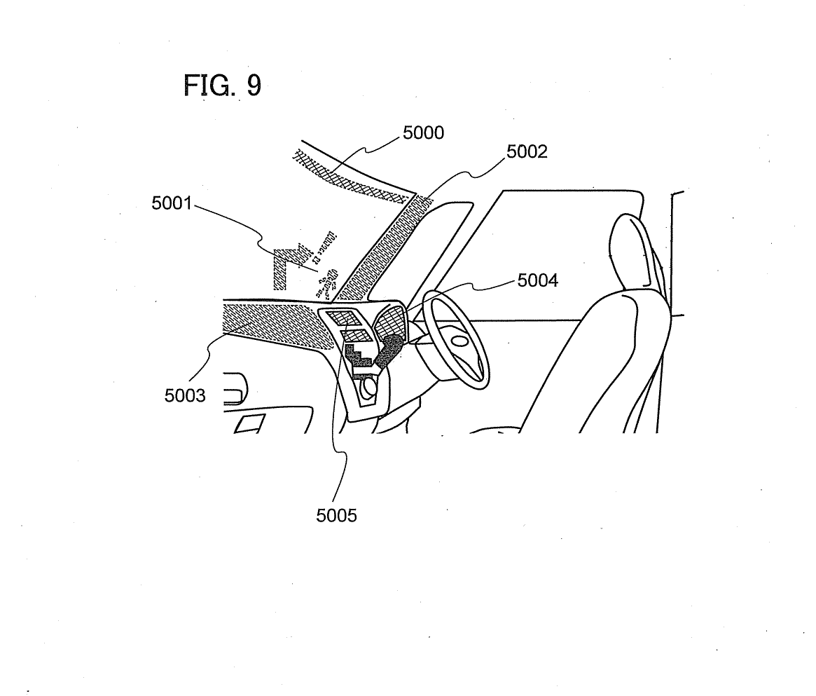

The light-emitting element including any of the carbazole compounds described in Embodiment 1 can also be used for an automobile windshield or an automobile dashboard. FIG. 9 illustrates one mode in which the light-emitting elements including any of the carbazole compounds described in Embodiment 1 are used for an automobile windshield and an automobile dashboard. Display regions 5000 to 5005 are each provided with a display device incorporating the light-emitting element including any of the carbazole compounds described in Embodiment 1.

The display region 5000 and the display region 5001 are each provided with the display device incorporating the light-emitting element which includes any of the carbazole compounds described in Embodiment 1 and which is provided in the automobile windshield. The light-emitting element including any of the carbazole compounds described in Embodiment 1 can be formed into a so-called see-through display device, through which the opposite side can be seen, by including a first electrode and a second electrode formed of electrodes having light-transmitting properties. Such see-through display devices can be provided even in the windshield of the car, without hindering the vision. Note that in the case where a transistor for driving the light-emitting element is provided, a transistor having a light-transmitting property, such as an organic transistor using an organic semiconductor material or a transistor using an oxide semiconductor, is preferably used.

The display region 5002 is a display device which is provided in a pillar portion and in which the light-emitting element including a carbazole compound described in Embodiment 1 is incorporated. The display region 5002 can compensate for the view hindered by the pillar portion by showing an image taken by an imaging unit provided in the car body. Similarly, the display region 5003 provided in the dashboard can compensate for the view hindered by the car body by showing an image taken by an imaging unit provided in the outside of the car body, which leads to elimination of blind areas and enhancement of safety. Showing an image so as to compensate for the area which a driver cannot see makes it possible for the driver to confirm safety easily and comfortably.

The display region 5004 and the display region 5005 can provide a variety of kinds of information such as navigation data, a speedometer, a tachometer, a mileage, a fuel meter, a gearshift indicator, and air-condition setting. The content or layout of the display can be changed freely by a user as appropriate. Note that such information can also be shown by the display regions 5000 to 5003. The display regions 5000 to 5005 can also be used as lighting devices.

By including any of the carbazole compounds described in Embodiment 1, the light-emitting element including the carbazole compound has low driving voltage and lower power consumption. Therefore, load on a battery is small even when a number of large screens such as the display regions 5000 to 5005 are provided, which provides comfortable use. For that reason, the light-emitting device and the lighting device each of which includes the light-emitting element including any of the carbazole compounds described in Embodiment 1 can be suitably used as an in-vehicle light-emitting device and lighting device.

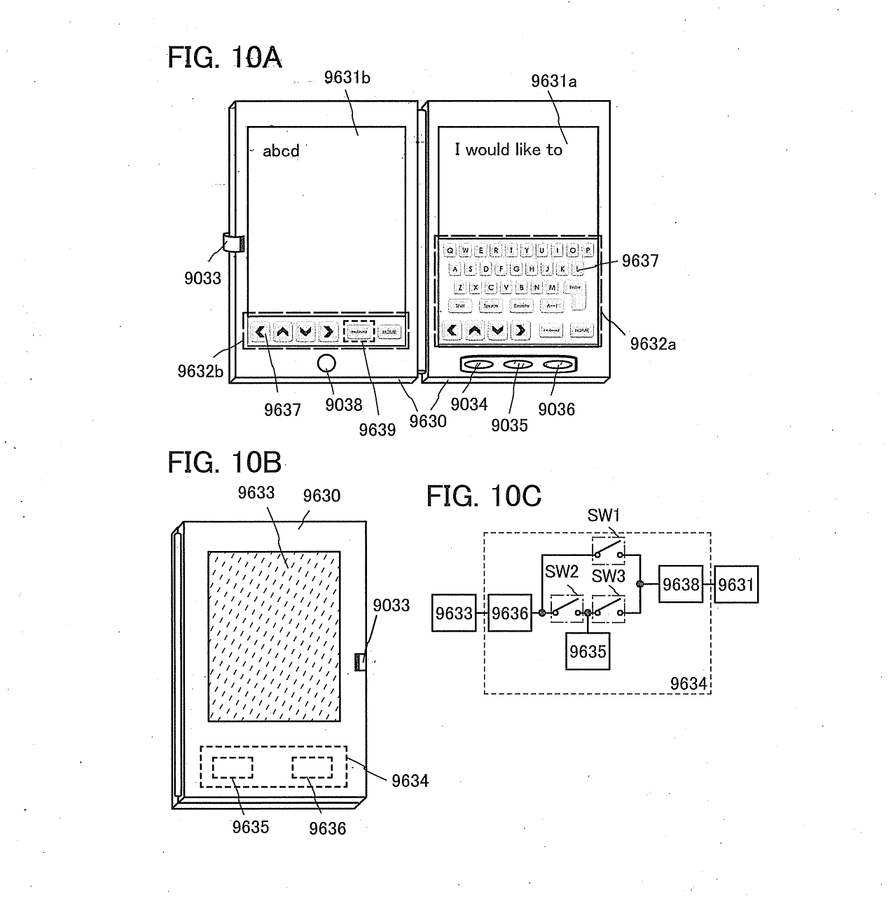

FIGS. 10A and 10B illustrate an example of a foldable tablet. FIG. 10A illustrates the tablet which is unfolded. The tablet includes a housing 9630, a display portion 9631a, a display portion 9631b, a display mode switch 9034, a power switch 9035, a power-saving mode switch 9036, a clasp 9033, and an operation switch 9038. Note that in the tablet, one or both of the display portion 9631a and the display portion 9631b is/are formed using a light-emitting device which includes a light-emitting element including any of the carbazole compounds described in Embodiment 1.

Part of the display portion 9631a can be a touchscreen region 9632a and data can be input when a displayed operation key 9637 is touched. Although half of the display portion 9631 a has only a display function and the other half has a touchscreen function, one embodiment of the present invention is not limited to the structure. The whole display portion 9631a may have a touchscreen function. For example, a keyboard is displayed on the entire region of the display portion 9631a so that the display portion 9631a is used as a touchscreen; thus, the display portion 9631b can be used as a display screen.

Like the display portion 9631a, part of the display portion 9631b can be a touchscreen region 9632b. When a switching button 9639 for showing/hiding a keyboard on the touchscreen is touched with a finger, a stylus, or the like, the keyboard can be displayed on the display portion 9631b.

Touch input can be performed in the touchscreen region 9632a and the touchscreen region 9632b at the same time.

The display mode switch 9034 can switch the display between portrait mode, landscape mode, and the like, and between monochrome display and color display, for example. The power-saving switch 9036 can control display luminance in accordance with the amount of external light in use of the tablet detected by an optical sensor incorporated in the tablet. Another detection device including a sensor for detecting inclination, such as a gyroscope or an acceleration sensor, may be incorporated in the tablet, in addition to the optical sensor.

Although FIG. 10A illustrates an example in which the display portion 9631a and the display portion 9631b have the same display area, one embodiment of the present invention is not limited to the example. The display portion 9631a and the display portion 9631b may have different display areas and different display quality. For example, one display panel may be capable of higher-definition display than the other display panel.

FIG. 10B illustrates the tablet which is folded. The tablet includes the housing 9630, a solar cell 9633, a charge and discharge control circuit 9634, a battery 9635, and a DC-to-DC converter 9636. As an example, FIG. 10B illustrates the charge and discharge control circuit 9634 including the battery 9635 and the DC-to-DC converter 9636.

Since the tablet is foldable, the housing 9630 can be closed when the tablet is not in use. As a result, the display portion 9631a and the display portion 9631b can be protected, thereby providing a tablet with high endurance and high reliability for long-term use.

The tablet illustrated in FIGS. 10A and 10B can have other functions such as a function to display various kinds of data (e.g., a still image, a moving image, and a text image), a function to display a calendar, a date, the time, or the like on the display portion, a touch-input function to operate or edit the data displayed on the display portion by touch input, and a function to control processing by various kinds of software (programs).

The solar cell 9633 provided on a surface of the tablet can supply power to the touchscreen, the display portion, a video signal processing portion, or the like. Note that the solar cell 9633 is preferably provided on one or two surfaces of the housing 9630, in which case the battery 9635 can be charged efficiently.

The structure and operation of the charge and discharge control circuit 9634 illustrated in FIG. 10B will be described with reference to a block diagram of FIG. 10C. FIG. 10C illustrates the solar cell 9633, the battery 9635, the DC-to-DC converter 9636, a converter 9638, switches SW1 to SW3, and the display portion 9631. The battery 9635, the DC-to-DC converter 9636, the converter 9638, and the switches SW1 to SW3 correspond to the charge and discharge control circuit 9634 illustrated in FIG. 10B.

First, description is made on an example of the operation in the case where power is generated by the solar cell 9633 with the use of external light. The voltage of the power generated by the solar cell is raised or lowered by the DC-to-DC converter 9636 so as to be voltage for charging the battery 9635. Then, when power supplied from the battery 9635 charged by the solar cell 9633 is used for the operation of the display portion 9631, the switch SW1 is turned on and the voltage of the power is raised or lowered by the converter 9638 so as to be voltage needed for the display portion 9631. When images are not displayed on the display portion 9631, the switch SW1 is turned off and the switch SW2 is turned on so that the battery 9635 is charged.

Although the solar cell 9633 is described as an example of a power generation means, the power generation means is not particularly limited, and the battery 9635 may be charged by another power generation means such as a piezoelectric element or a thermoelectric conversion element (Peltier element). The battery 9635 may be charged by a non-contact power transmission module which is capable of charging by transmitting and receiving power by wireless (without contact), or another charge means used in combination, and the power generation means is not necessarily provided.

Needless to say, one embodiment of the present invention is not limited to the electronic device having the shape illustrated in FIGS. 10A to 10C as long as the display portion 9631a or 9631b is included.

### EXAMPLE 1

In this example, a synthesis method of 1-[3,5-di(9H-carbazol-9-yl)phenyl]-2-phenylbenzimidazole (abbreviation: 1Cz2BIm, Structureal Formula (100)), which is the carbazole compound described in Embodiment 1 and represented by General Formula (G1), and physical properties thereof will be described. The structural formula of 1Cz2BIm is shown below.

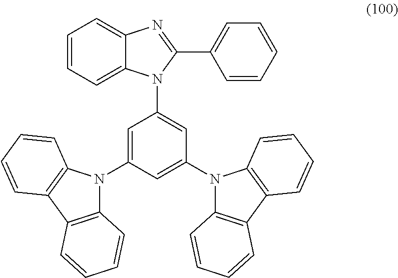

### <Synthesis Method>

### Step 1: Synthesis of 9-[3-(9H-Carbazol-9-yl)-5-chloro]phenyl-9H-carbazole (abbreviation: mCP-C1)

In a 200 mL three-neck flask, a mixture of 5.0 g (19 mmol) of 1,3-dibromo-5-chlorobenzene, 6.5 g (39 mmol) of carbazole, 370 mg (1.9 mmol) of copper iodide, 510 mg (1.94 mmol) of 18-crown-6-ether, 8.9 g (47 mmol) of potassium carbonate, and 20 mL of 1,3-dimethyl-3,4,5,6-tetrahydro-2-(1H)pyrimidinone was deaerated while being stirred under reduced pressure and then heated and stirred at 170° C. in a nitrogen atmosphere for 11 hours to cause a reaction.

After the reaction, this reaction mixture was washed with water, and an organic layer and an aqueous layer were separated. Then, magnesium sulfate was added to the organic layer to remove moisture. This suspension was filtered to obtain a filtrate. The obtained filtrate was concentrated and purified by silica gel column chromatography. A mixed solvent of toluene and hexane (toluene: hexane=1:5) was used as a developing solvent for the chromatography. The Rf value of the substance that was the object of the synthesis was 0.35, which was obtained by silica gel thin layer chromatography (TLC) (with a developing solvent containing ethyl acetate and hexane in a 10:1 ratio).

The obtained fraction was concentrated, and hexane was added thereto. The mixture was irradiated with ultrasonic waves and then recrystallized to give 6.6 g of white powder that was the object of the synthesis in a yield of 80%. A scheme of the synthesis of Step 1 is shown in (a-1).

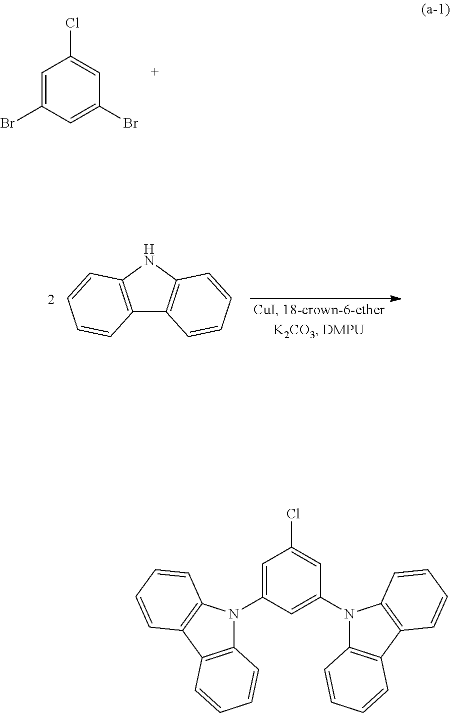 [SMILES: Clc1cc(Br)cc(Br)c1 ; c1ccc2c(c1)[nH]c1ccccc12 ; Clc1cc(-n2c3ccccc3c3ccccc32)cc(-n2c3ccccc3c3ccccc32)c1]

The compound obtained in Step 1 was subjected to a nuclear magnetic resonance (NMR) measurement. The measurement data are as follows: ¹H NMR (CDCl₃, 300 MHz): δ(ppm)=7.31-7.64 (m, 12H), 7.71-7.72 (m, 1H), 7.75-7.76 (m, 1H), 7.84-7.85 (m, 1H), 8.15(d, J=7.8 Hz, 4H).

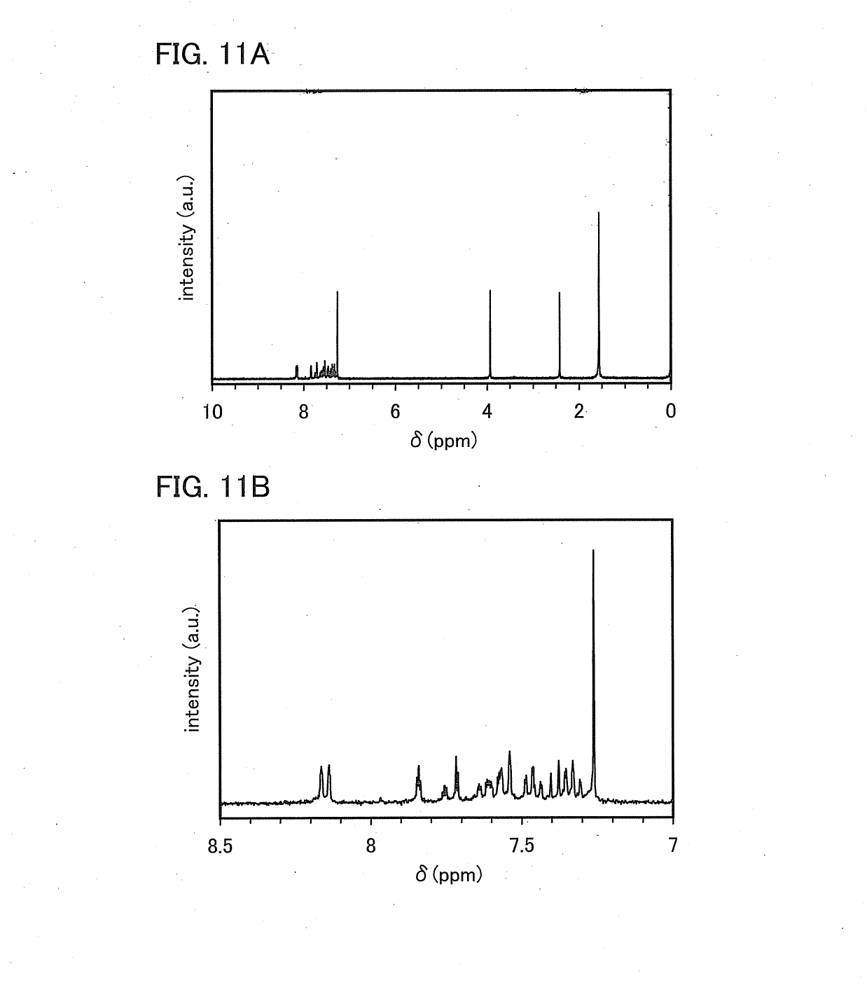

FIGS. 11A and 11B are ¹H NMR charts. Note that FIG. 11B is a chart where the range of from 7.00 ppm to 8.50 ppm in FIG. 11A is enlarged. The above results reveal that 9-[3-(9H-carbazol-9-yl)-5-chloro]phenyl-9H-carbazole (abbreviation: mCP-Cl) that was the object of the synthesis was obtained.

### Step 2: Synthesis of 1-[3,5-di(9H-Carbazol-9-yl)phenyl]-2-phenylbenzimidazole (abbreviation: 1Cz2BIm)

In a 200 mL three-neck flask, a mixture of 3.0 g (6.78 mmol) of 9-[3-(9H-carbazol-9-yl)-5-chloro]phenyl-9H-carbazole (abbreviation: mCP-C1), 1.2 g (6.2 mmol) of 2-phenylbenzimidazole, 22 mg (60 μmol) of allylpalladium(II)chloride dimer ([PdCl(C₃H₅)]₂), 84.6 mg (0.24 mmol) of di-tert-butyl(2,2-diphenyl-1-methyl-1-cyclopropyl)phosphine (abbreviation: cBRIDP), 710 mg (7.4 mmol) of sodium t-butoxide, and 30 mL of xylene was deaerated while being stirred under reduced pressure and then heated and stirred at 120° C. in a nitrogen atmosphere for 11 hours to cause a reaction. Then, to the reaction mixture solution were added 22 mg (60 μmol) of allylpalladium(II)chloride dimer ([PdCl(C₃H₅)]₂), 85 mg (0.24 mmol) of di-tert-butyl(2,2-diphenyl-1-methyl-1-cyclopropyl)phosphine (abbreviation: cBRIDP), and 710 mg (7.39 mmol) of sodium t-butoxide, and the mixture was heated and stirred at 120° C. in a nitrogen atmosphere for 7 hours to cause a reaction. Furthermore, 22 mg (60 μmol) of allylpalladium(II)chloride dimer ([PdCl(C₃H₅)]₂) was added to the reaction mixture, and the mixture was heated and stirred at 120° C. in a nitrogen atmosphere for 5 hours to cause a reaction. Lastly, to the reaction mixture solution were added 22 mg (60 μmol) of allylpalladium(II)chloride dimer ([PdCl(C₃H₅)]₂) and 85 mg (0.24 mmol) of di-tert-butyl(2;2-diphenyl-1-methyl-1-cyclopropyl)phosphine, and the mixture was deaerated while being stirred under reduced pressure. After that, the mixture was heated and stirred at 120° C. in a nitrogen atmosphere for 6 hours to cause a reaction.

After the reaction, 2.0 L of toluene was added to the reaction mixture solution, and an organic layer of the mixture solution was filtered through Florisil (Catalog No. 540-00135, produced by Wako Pure Chemical Industries, Ltd.), alumina (neutral, produced by Merck Ltd.), and Celite (Catalog No. 531-16855, produced by Wako Pure Chemical Industries, Ltd.). The obtained filtrate was concentrated and purified by silica gel column chromatography. A mixed solvent of toluene and hexane (toluene: hexane=10:1) was used as a developing solvent for the chromatography. The Rf value of the substance that was the object of the synthesis was 0.05, which was obtained by silica gel thin layer chromatography (TLC) (with a developing solvent of toluene).

The obtained fraction was concentrated and then recrystallized from hexane to give 1.9 g of white powder that was the object of the synthesis in a yield of 51%. A scheme of the synthesis Step 2 is shown in (a-2).

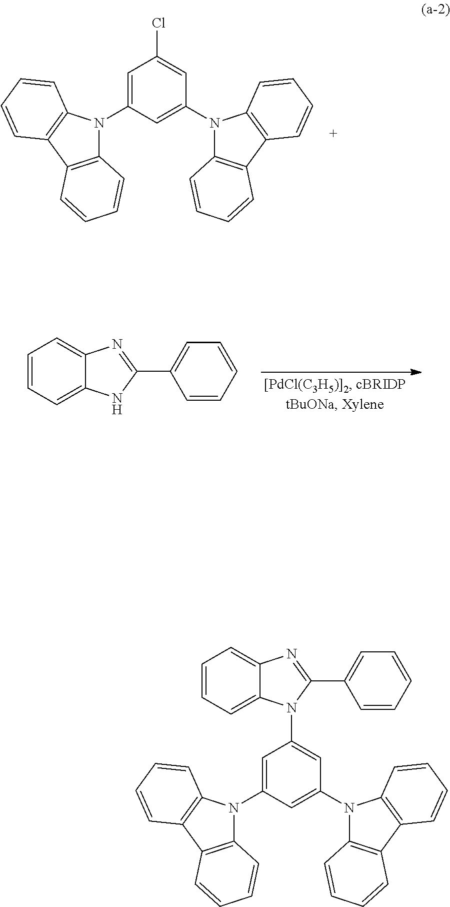 [SMILES: Clc1cc(-n2c3ccccc3c3ccccc32)cc(-n2c3ccccc3c3ccccc32)c1 ; c1ccc(-c2nc3ccccc3[nH]2)cc1 ; c1ccc(-c2nc3ccccc3n2-c2cc(-n3c4ccccc4c4ccccc43)cc(-n3c4ccccc4c4ccccc43)c2)cc1]

A compound obtained in Step 2 was subjected to a nuclear magnetic resonance (NMR) measurement. The measurement data are as follows: ¹H NMR (CDCl₃, 300 MHz): δ(ppm)=7.23-7.43 (m, 14H), 7.56-7.66 (m, 6H), 7.79-7.81 (m, 2H), 7.91-7.97 (m, 2H), 8.13 (d, J=6.8 Hz, 4H).

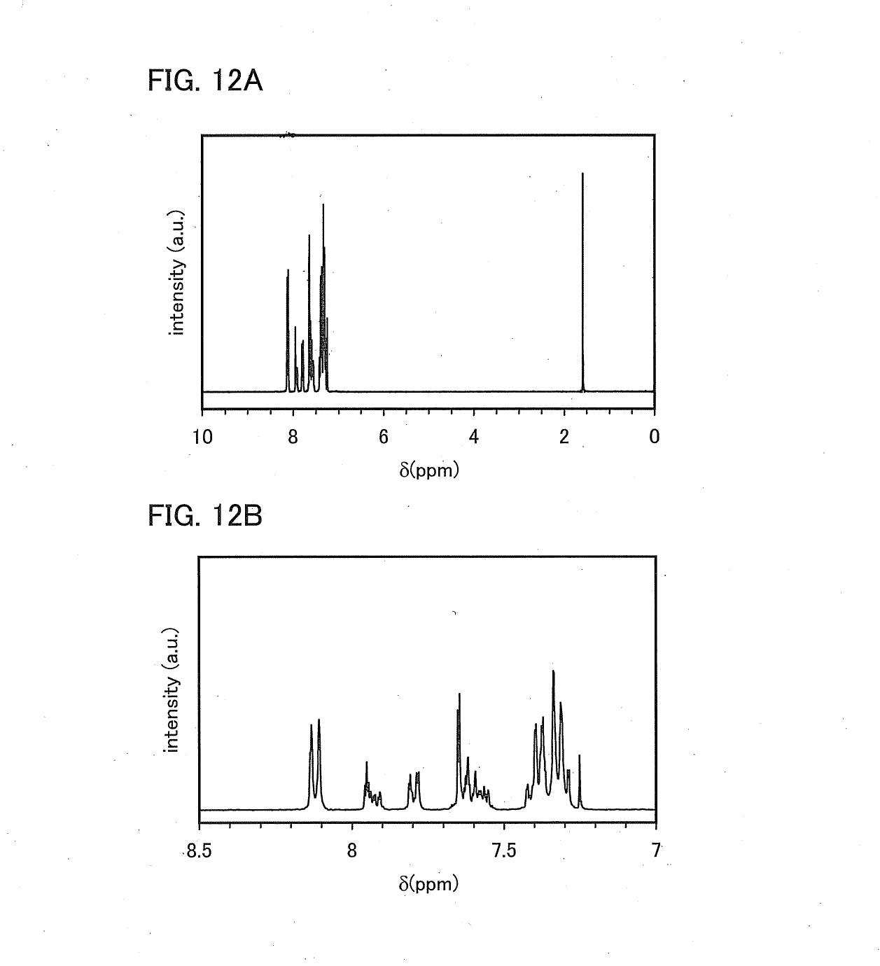

FIGS. 12A and 12B are ¹H NMR charts. Note that FIG. 12B is a chart where the range of from 7.00 ppm to 8.50 ppm in FIG. 12A is enlarged. The above results reveal that 1-[3,5-di(9H-carbazol-9-yl)phenyl]-2-phenylbenzimidazole (abbreviation: 1Cz2BIm) that was the object of the synthesis was obtained.

### <<Physical Properties of 1Cz2BIm>>

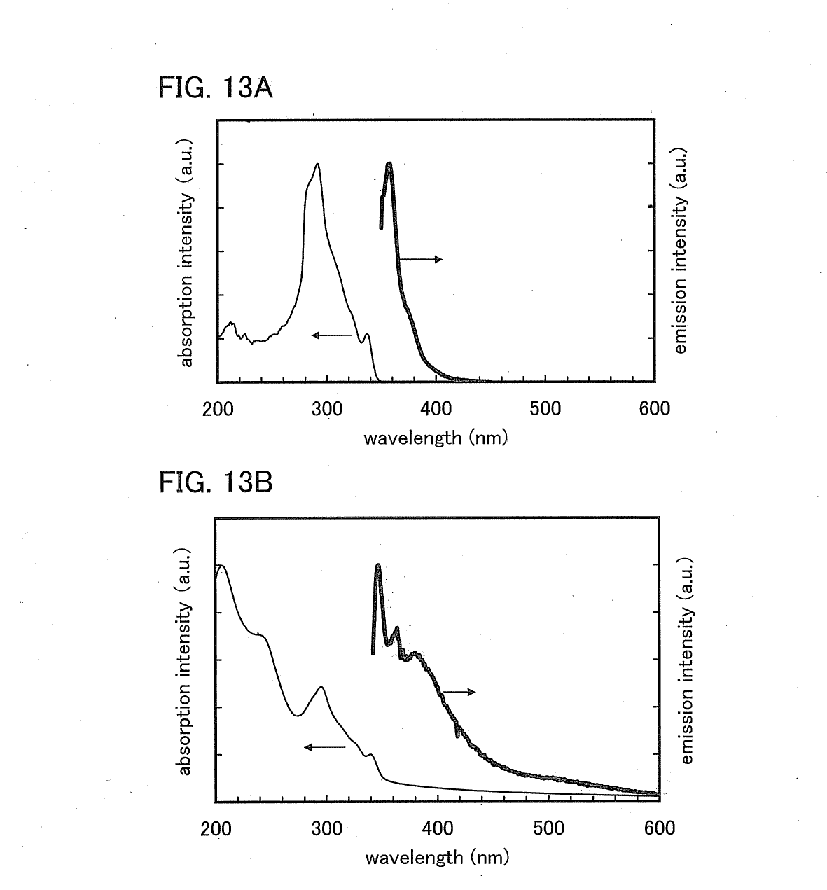

FIG. 13A shows an absorption and emission spectra of 1Cz2BIm in a toluene solution of 1Cz2BIm and FIG. 13B shows an absorption and emission spectra of a thin film of 1Cz2BIm. An ultraviolet-visible spectrophotometer (V-550, produced by JASCO Corporation) was used for measurement of the spectra. The spectra of the toluene solution were measured with a toluene solution of 1Cz2BIm put in a quartz cell. The spectra of the thin film were measured with a sample prepared by deposition of 1Cz2BIm on a quartz substrate by evaporation. Note that in the case of the absorption spectrum of 1Cz2BIm in the toluene solution of 1Cz2BIm, the absorption spectrum obtained by subtraction of the absorption spectra of quartz and toluene from the measured spectra is shown in the drawing and that in the case of the absorption spectrum of the thin film of 1Cz2BIm, the absorption spectrum obtained by subtraction of the absorption spectrum of the quartz substrate from the measured spectra is shown in the drawing.

FIG. 13A shows that the absorption peak wavelengths of 1Cz2BIm in the toluene solution of 1Cz2BIm are around 337 nm and 290 nm and that the emission peak wavelength thereof is around 358 nm (excitation wavelength: 313 nm). FIG. 13B shows that the absorption peak wavelengths of the thin film of 1Cz2BIm are around 340 nm, 324 nm, 307 nm, 295 nm, 240 nm, and 206 nm and that the emission peak wavelengths thereof are around 380 nm, 364 nm, and 347 nm (excitation wavelength: 340 nm). The above results show that 1Cz2BIm emits light with a very short wavelength.

Further, the ionization potential of 1Cz2BIm in a thin film state was measured by a photoelectron spectrometer (AC-2, produced by Riken Keiki, Co., Ltd.) in air. The obtained value of the ionization potential was converted into a negative value to give a HOMO level of 1Cz2BIm of −5.87 eV. From the data of the absorption spectrum of the thin film of 1Cz2BIm in FIG. 13B, the absorption edge of 1Cz2BIm, which was obtained from a Tauc plot with an assumption of direct transition, was 3.53 eV. Therefore, the optical energy gap of 1Cz2BIm in a solid state can be estimated at 3.53 eV; from the values of the HOMO level obtained above and this energy gap, the LUMO level of 1Cz2BIm can be estimated at −2.34 eV. This reveals that 1Cz2BIm in the solid state has an energy gap as wide as 3.53 eV.

### EXAMPLE 2

In this example, description will be made on a light-emitting element in which 1-[3,5-di(9H-carbazol-9-yl)phenyl]-2-phenylbenzimidazole (abbreviation: 1Cz2BIm, Structural Formula (100)), the carbazole compound described in Embodiment 1, is used as a host material in a light-emitting layer containing an emission center substance emitting green phosphorescence.

Molecular structures of organic compounds used in this example are shown in Structural Formulae (i) to (vi) and (100). The element structure was similar to that illustrated in FIG. 1A.

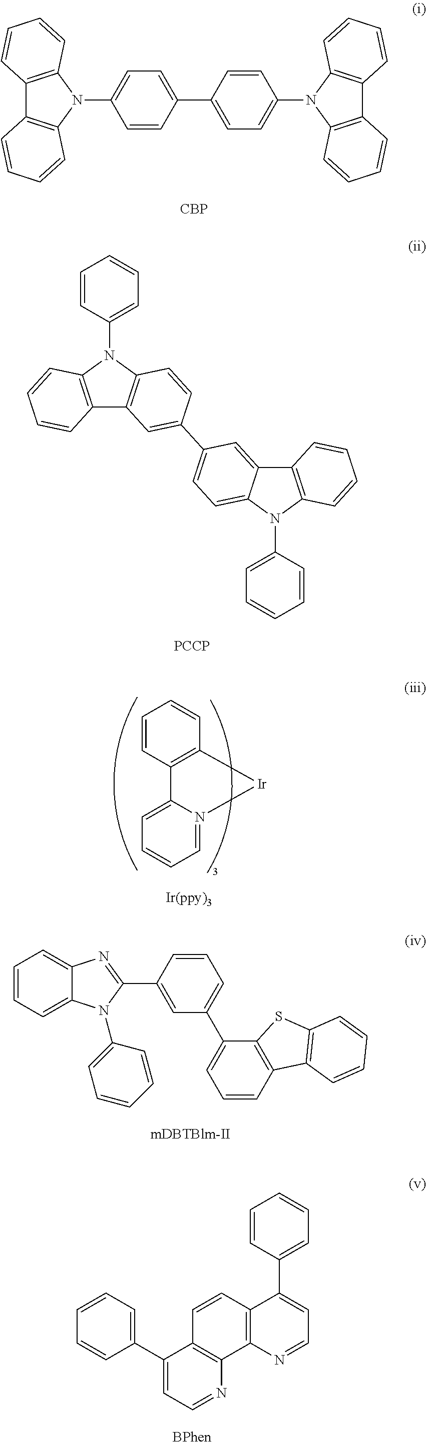

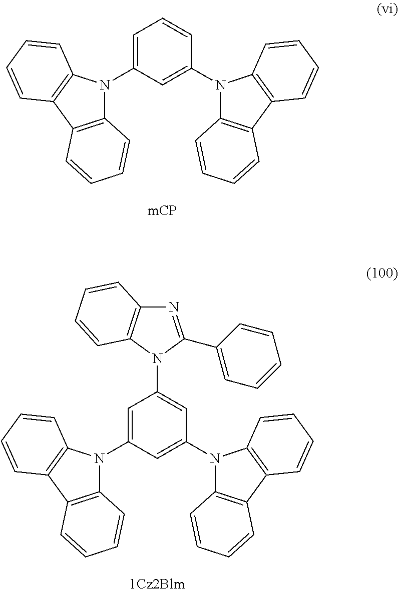 [SMILES: c1cc(-n2c3ccccc3c3ccccc32)cc(-n2c3ccccc3c3ccccc32)c1 ; c1ccc(-c2nc3ccccc3n2-c2cc(-n3c4ccccc4c4ccccc43)cc(-n3c4ccccc4c4ccccc43)c2)cc1]

### <<Manufacture of Light-Emitting Element 1 and Comparative Light-Emitting Element 1>>

First, a glass substrate, over which a film of indium tin oxide containing silicon (ITSO) was formed to a thickness of 110 nm as the first electrode 101, was prepared. A surface of the ITSO film was covered with a polyimide film so that an area of 2 mm×2 mm of the surface was exposed. The electrode area was 2 mm×2 mm. As pretreatment for forming the light-emitting element over the substrate, the surface of the substrate was washed with water and baked at 200° C. for 1 hour, and then was subjected to UV ozone treatment for 370 seconds. After that, the substrate was transferred into a vacuum evaporation apparatus where the pressure had been reduced to approximately 10⁻⁴ Pa, and was subjected to vacuum baking at 170° C. for 30 minutes in a heating chamber of the vacuum evaporation apparatus, and then the substrate was cooled down for about 30 minutes.

Next, the substrate was fixed to a holder provided in the vacuum evaporation apparatus so that the surface provided with the ITSO film faced downward.

After the pressure in the vacuum evaporation apparatus was reduced to 10⁻⁴ Pa, 4,4′-bis(N-carbazolyl)biphenyl (abbreviation: CBP) represented by Structural Formula (i) and molybdenum(VI) oxide were deposited by co-evaporation so that the weight ratio of CBP to molybdenum oxide was 2:1, whereby a hole-injection layer 111 was formed. The thickness of the hole-injection layer 111 was 60 nm. Note that co-evaporation is an evaporation method in which a plurality of different substances are vaporized from the respective different evaporation sources at the same time.

Next, a film of 9-phenyl-9H-3-(9-phenyl-9H-carbazol-3-yl)carbazole (abbreviation: PCCP) represented by Structural Foimula (ii) was formed to a thickness of 20 nm by evaporation, whereby a hole-transport layer 112 was formed.

Further, on the hole-transport layer 112, a film containing 1-[3,5-di(9H-carbazol-9-yl)phenyl]-2-phenylbenzimidazole (abbreviation: 1Cz2BIm), which is the carbazole compound described in Embodiment 1 and represented by Structural Formula (100), PCCP, and tris(2-phenylpyridine)iridium (abbreviation::[Ir(ppy)₃]) represented by Structural Formula (iii) in a 1:0.3:0.08 weight ratio was formed to a thickness of 30 nm by evaporation, whereby a light-emitting layer 113 was formed.

Next, a film of 2-[3-(dibenzothiophen-4-yl)phenyl]-1-phenyl-1H-benzimidazole (abbreviation: mDBTBIm-II) represented by Structural Formula (iv) was formed to a thickness of 10 nm by evaporation, and then a film of bathophenanthroline (abbreviation: BPhen) represented by Structural Formula (v) was formed to a thickness of 15 nm by evaporation, whereby an electron-transport layer 114 was formed.

Further, a film of lithium fluoride was formed to a thickness of 1 nm on the electron-transport layer 114 by evaporation, whereby an electron-injection layer 115 was formed. Lastly, a film of aluminum was formed to a thickness of 200 nm as a second electrode 102 which serves as a cathode. Thus, the light-emitting element 1 was completed. Note that the above evaporation steps were all performed by a resistance-heating method.

In manufacture of the comparative light-emitting element 1, a light-emitting layer 113 was formed of a co-evaporation film of mCP and [Ir(ppy)₃], which is different from the light-emitting layer 113 in the light-emitting element 1. In other words, the light-emitting layer 113 in the comparative light-emitting element 1 was formed to a thickness of 30 nm by co-evaporation of mCP and [Ir(ppy)₃] in a 1:0.08 weight ratio.

### <<Operation Characteristics of Light-Emitting Element 1 and Comparative Light-Emitting Element 1>>

The thus obtained light-emitting element 1 and comparative light-emitting element 1 were put in a glove box in a nitrogen atmosphere, and the light-emitting elements were sealed so as not to be exposed to the air. Then, the operation characteristics of these light-emitting elements were measured. Note that the measurement was carried out at room temperature (in an atmosphere kept at 25° C.).

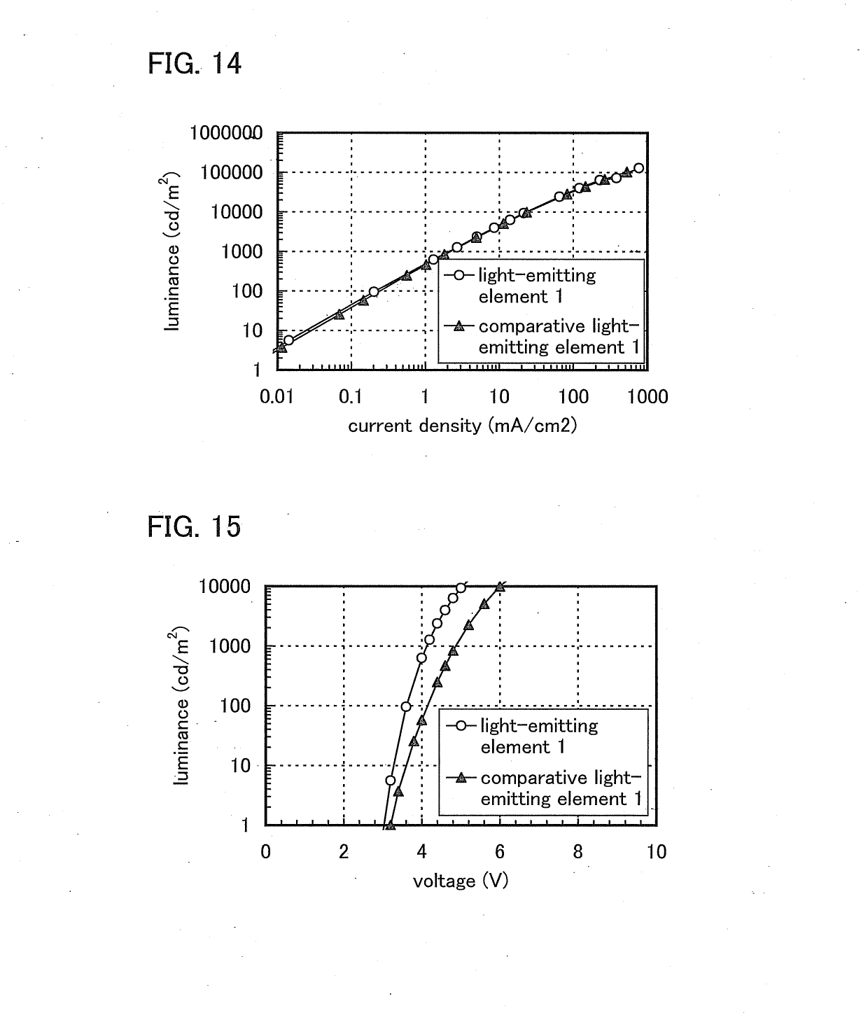

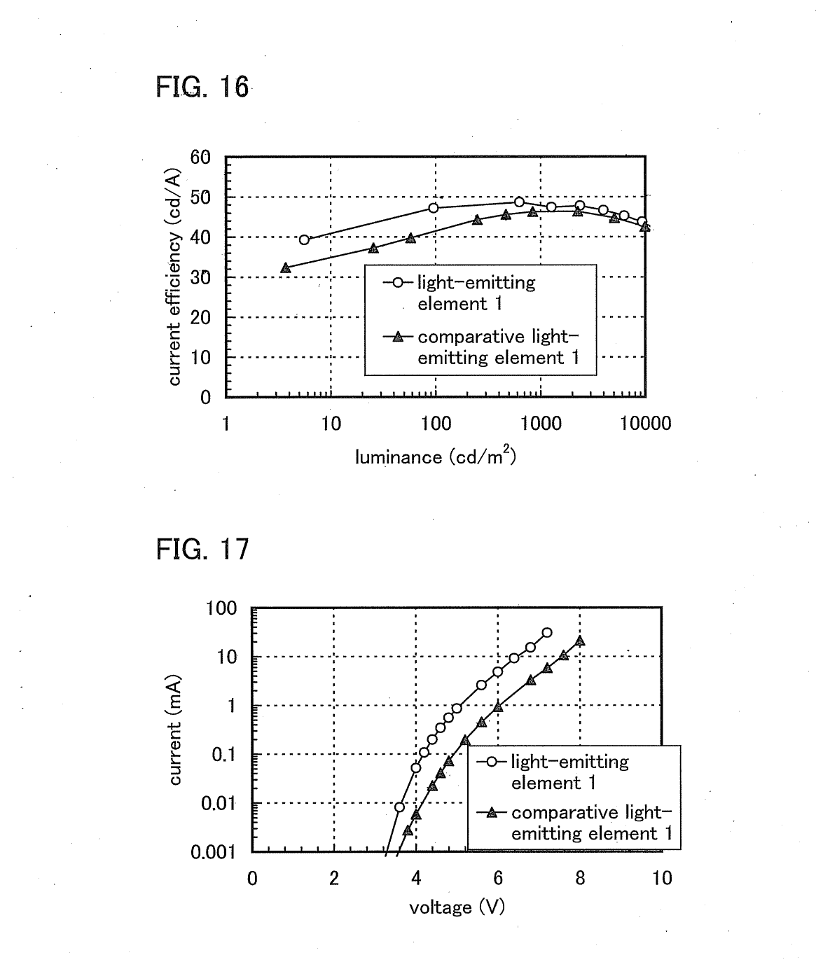

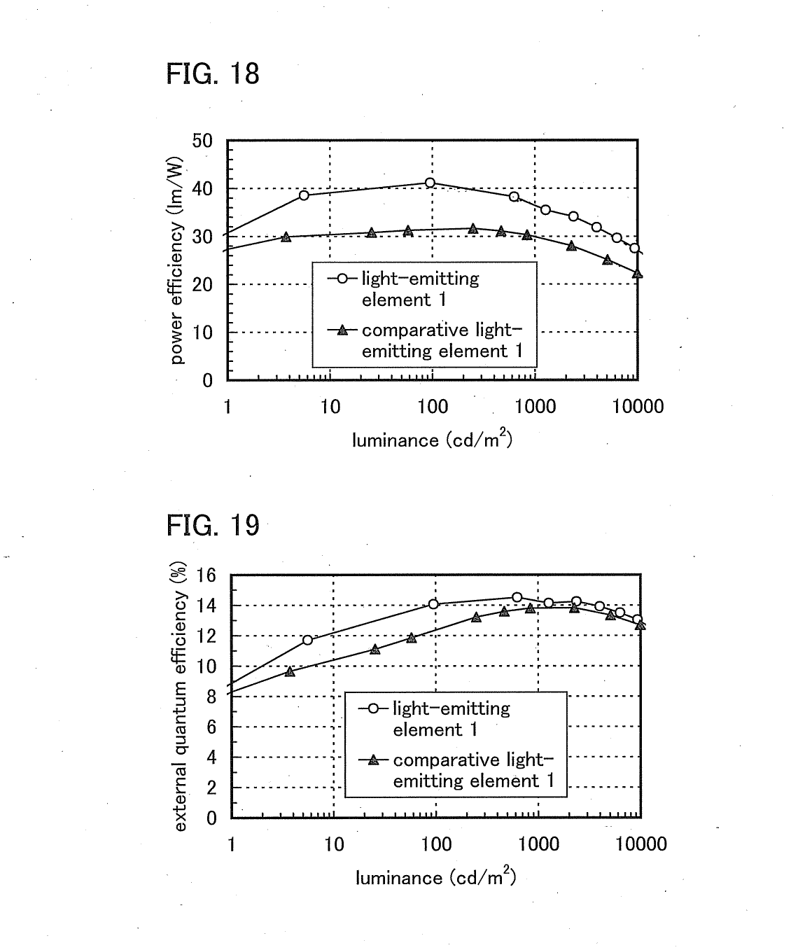

FIG. 14 shows luminance versus current density characteristics of the light-emitting element 1 and the comparative light-emitting element 1. FIG. 15 shows luminance versus voltage characteristics thereof FIG. 16 shows current efficiency versus luminance characteristics thereof FIG. 17 shows current versus voltage characteristics thereof. FIG. 18 shows power efficiency versus luminance characteristics thereof FIG. 19 shows external quantum efficiency versus luminance characteristics thereof. In FIG. 14, the vertical axis represents luminance (cd/m²) and the horizontal axis represents current density (mA/cm²). In FIG. 15, the vertical axis represents luminance (cd/m²) and the horizontal axis represents voltage (V). In FIG. 16, the vertical axis represents current efficiency (cd/A) and the horizontal axis represents luminance (cd/m²). In FIG. 17, the vertical axis represents current (mA) and the horizontal axis represents voltage (V). In FIG. 18, the vertical axis represents power efficiency (lm/W) and the horizontal axis represents luminance (cd/m²). In FIG. 19, the vertical axis represents external quantum efficiency (%) and the horizontal axis represents luminance (cd/m²).

FIG. 16 shows that the light-emitting element 1, in which the carbazole compound represented by General Formula (G1) was used as the host material in the light-emitting layer of the light-emitting element emitting green phosphorescence, has better current efficiency versus luminance characteristics and higher emission efficiency than the comparative light-emitting element 1, in which mCP was used as a host material in the same way. This is partly because the carbazole compound represented by General Formula (G1) has as a high triplet excitation level and as a wide energy gap as mCP, which allows even a light-emitting substance emitting green phosphorescence to be effectively excited. In addition, FIG. 15 shows that the light-emitting element, in which the carbazole compound represented by General Formula (G1) was used as the host material in the light-emitting layer of the light-emitting element emitting green phosphorescence, has very good luminance versus voltage characteristics and low driving voltage. This reveals that the carbazole compound represented by General Formula (G1) has a high carrier-transport property. FIG. 14 similarly shows that the light-emitting element 1 has better luminance versus current density characteristics than the comparative light-emitting element 1. Moreover, as shown in FIG. 19, the light-emitting element 1 also has high external quantum efficiency.

As described above, the light-emitting element including the carbazole compound represented by General Formula (G1) has good characteristics such as high emission efficiency and low driving voltage. Thus, as shown in FIG. 18, the power efficiency versus luminance characteristics of the light-emitting element including the carbazole compound represented by General Formula (G1) is much better than those of the comparative light-emitting element.

Note that mCP, which was used for comparison, has a wide energy gap and a high triplet excitation level and thus is often used as a host material in an element emitting short-wavelength phosphorescence and is known to allow a phosphorescent light-emitting element having high emission efficiency to be manufactured. It was found that the carbazole compound described in Embodiment 1 makes it possible to obtain a light-emitting element having much higher emission efficiency than the light-emitting element including mCP.

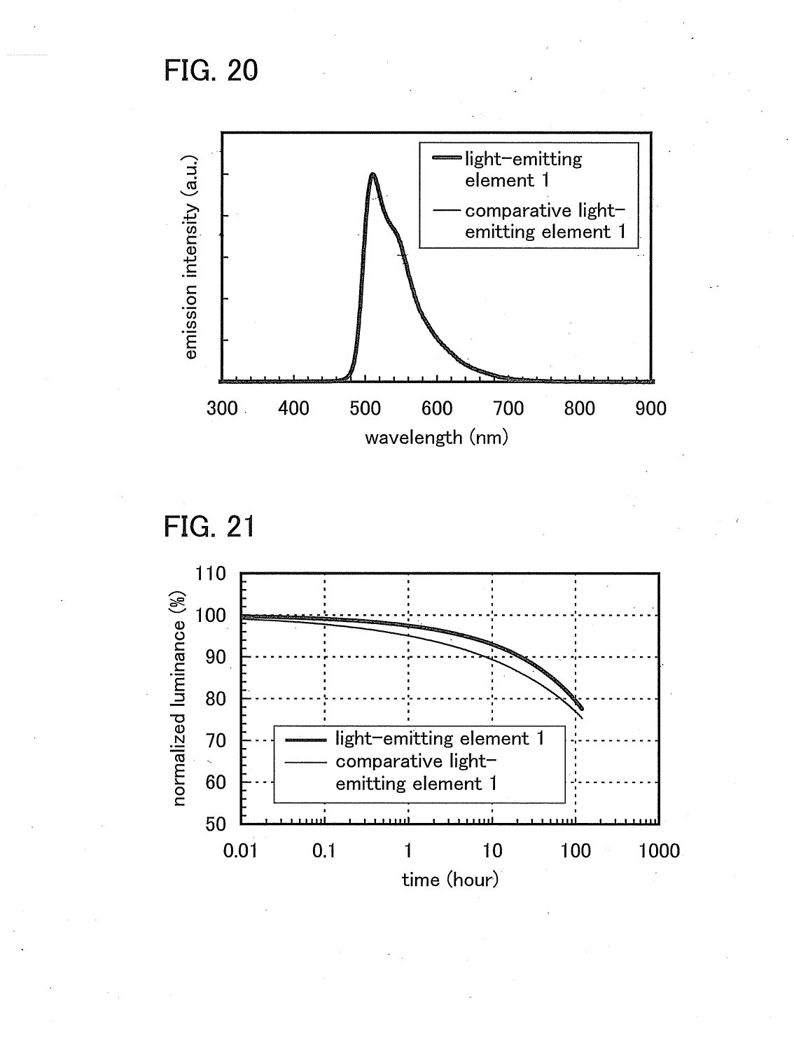

FIG. 20 shows emission spectra of the manufactured light-emitting element 1 and comparative light-emitting element 1 when a current of 0.1 mA was made to flow through each of the light-emitting elements. In FIG. 20, the vertical axis represents emission intensity (arbitrary unit) and the horizontal axis represents wavelength (nm). The emission intensity is shown as a value relative to the maximum emission intensity assumed to be 1. According to FIG. 20, the emission spectra of the light-emitting element 1 and the comparative light-emitting element 1 overlap, and both the light-emitting element 1 and the comparative light-emitting element 1 emit green light emanating from [Ir(ppy)₃], which is the emission center substance.

Next, with an initial luminance set to 1000 cd/m², these elements were driven under a condition where the current density was constant, and changes in luminance relative to driving time were examined. FIG. 21 shows normalized luminance versus time characteristics. FIG. 21 shows that the light-emitting element 1 has characteristics equivalent to or better than those of the comparative light-emitting element 1 and high reliability.

### EXAMPLE 3

In this example, description will be, made on a light-emitting element (light-emitting element 2) in which 1-[3,5-di(9H-carbazol-9-yl)phenyl]-2-phenylbenzimidazole (abbreviation: 1Cz2BIm, Structural Formula (100)), the carbazole compound described in Embodiment 1, is used as a host material in a light-emitting layer containing an emission center substance emitting blue-green phosphorescence.

Molecular structures of organic compounds used in this example are shown in Structural Formulae (i), (ii), (iv) to (vii), and (100). The element structure was similar to that illustrated in FIG. 1A.

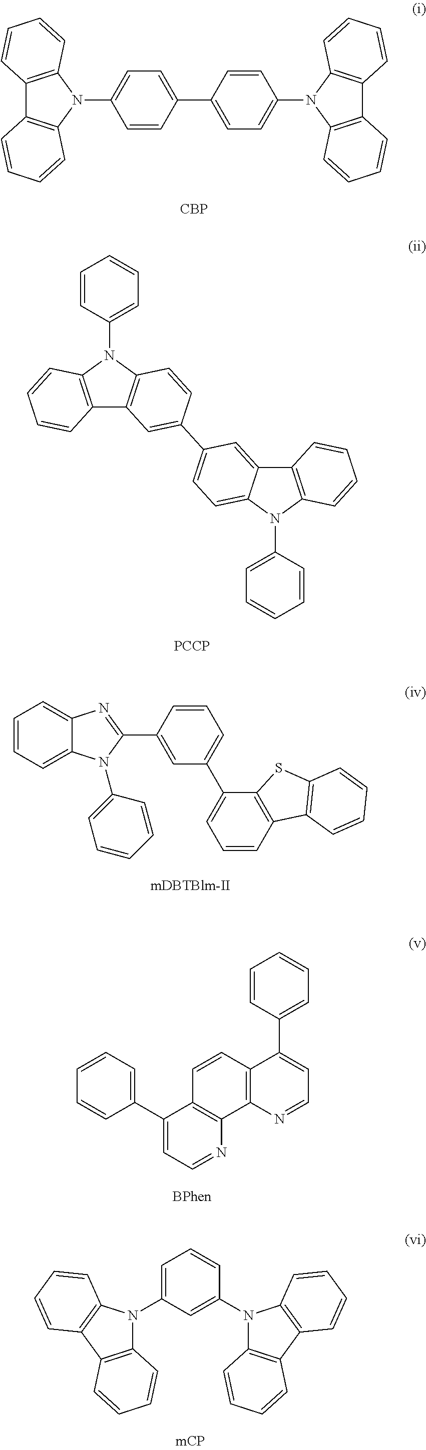

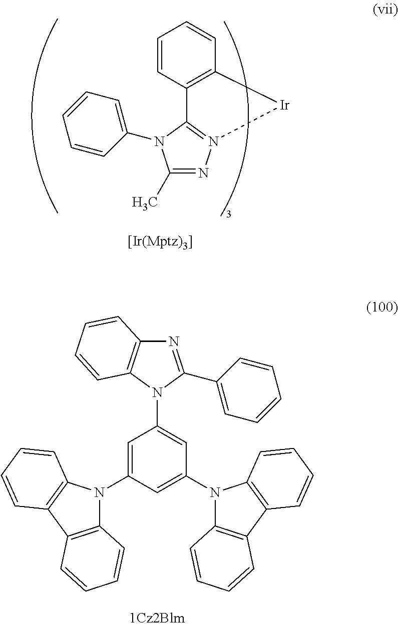

<<Manufacture of Light-Emitting Element 2 and Comparative. Light-Emitting Element 2>>

First, a glass substrate, over which a film of indium tin oxide containing silicon (ITSO) was formed to a thickness of 110 nm as the first electrode 101, was prepared. A surface of the ITSO film was covered with a polyimide film so that an area of 2 mm×2 mm of the surface was exposed. The electrode area was 2 mm×2 mm. As pretreatment for forming the light-emitting element over the substrate, the surface of the substrate was washed with water and baked at 200° C. for 1 hour, and then was subjected to UV ozone treatment for 370 seconds. After that, the substrate was transferred into a vacuum evaporation apparatus where the pressure had been reduced to approximately 10⁻⁴ Pa, and was subjected to vacuum baking at 170° C. for 30 minutes in a heating chamber of the vacuum evaporation apparatus, and then the substrate was cooled down for about 30 minutes.

Next, the substrate was fixed to a holder provided in the vacuum evaporation apparatus so that the surface provided with the ITSO film faced downward.

After the pressure in the vacuum evaporation apparatus was reduced to 10⁻⁴ Pa, 4,4′-bis(N-carbazolyl)biphenyl (abbreviation: CBP) represented by Structural Formula (i) and molybdenum(VI) oxide were deposited by co-evaporation so that the weight ratio of CBP to molybdenum oxide was 2:1, whereby a hole-injection layer 111 was Ruined. The thickness of the hole-injection layer 111 was 60 nm. Note that co-evaporation is an evaporation method in which a plurality of different substances are vaporized from the respective different evaporation sources at the same time.

Next, a film of 1,3-bis(N-carbazolyl)benzene (abbreviation: mCP) represented by Structural Formula (vi) was formed to a thickness of 20 nm by evaporation, whereby a hole-transport layer 112 was formed.

Further, on the hole-transport layer 112, a film containing 1-[3,5-di(9H-carbazol-9-yl)phenyl]-2-phenylbenzimidazole (abbreviation: 1Cz2BIm), which is the carbazole compound described in Embodiment 1 and represented by Structural Formula (100), 9-phenyl-9H-3-(9-phenyl-9H-carbazol-3-yl)carbazole (abbreviation: PCCP) represented by Structural. Formula (ii), and tris(5-methyl-3 ,4-diphenyl-4H-1,2,4-triazolato)iridium(III) (abbreviation: [Ir(Mptz)₃]) represented by Structural Formula (vii) in a 1:0.5:0.08 weight ratio was formed to a thickness of 30 nm by evaporation, and then a film containing 2-[3-(dibenzothiophen-4-yl)phenyl]-1-phenyl-1H-benzimidazole (abbreviation: mDB IBIm-II) represented by Structural Formula (iv) and [Ir(Mptz)₃] in a 1:0.08 weight ratio was formed to a thickness of 10 nm by evaporation, whereby a light-emitting layer 113 was formed.

Next, a film of bathophenanthroline (abbreviation: BPhen) represented by Structural Formula (v) was forme to a thickness of 20 nm by evaporation, whereby an electron-transport layer 114 was formed.

Further, a film of lithium fluoride was formed to a thickness of 1 nm on the electron-transport layer 114 by evaporation, whereby an electron-injection layer 115 was formed. Lastly, a film of aluminum was formed to a thickness of 200 nm as a second electrode 102 which serves as a cathode. Thus, the light-emitting element 2 was completed. Note that the above evaporation steps were all performed by a resistance-heating method.

In manufacture of the comparative light-emitting element 2, a light-emitting layer 113 was formed of a co-evaporation film of mCP and [Ir(Mptz)₃], which is different from the light-emitting layer 113 in the light-emitting element 2. In other words, the light-emitting layer 113 in the comparative light-emitting element 2 was formed to a thickness of 30 nm by co-evaporation of mCP and [Ir(Mptz)₃] in a 1:0.08 weight ratio.

### <<Operation Characteristics of Light-Emitting Element 2 and Comparative Light-Emitting Element 2>>

The thus obtained light-emitting element 2 and comparative light-emitting element 2 were put in a glove box in a nitrogen atmosphere, and the light-emitting elements were sealed so as not to be exposed to the air. Then, the operation characteristics of these light-emitting elements were measured. Note that the measurement was carried out at room temperature (in an atmosphere kept at 25° C.).

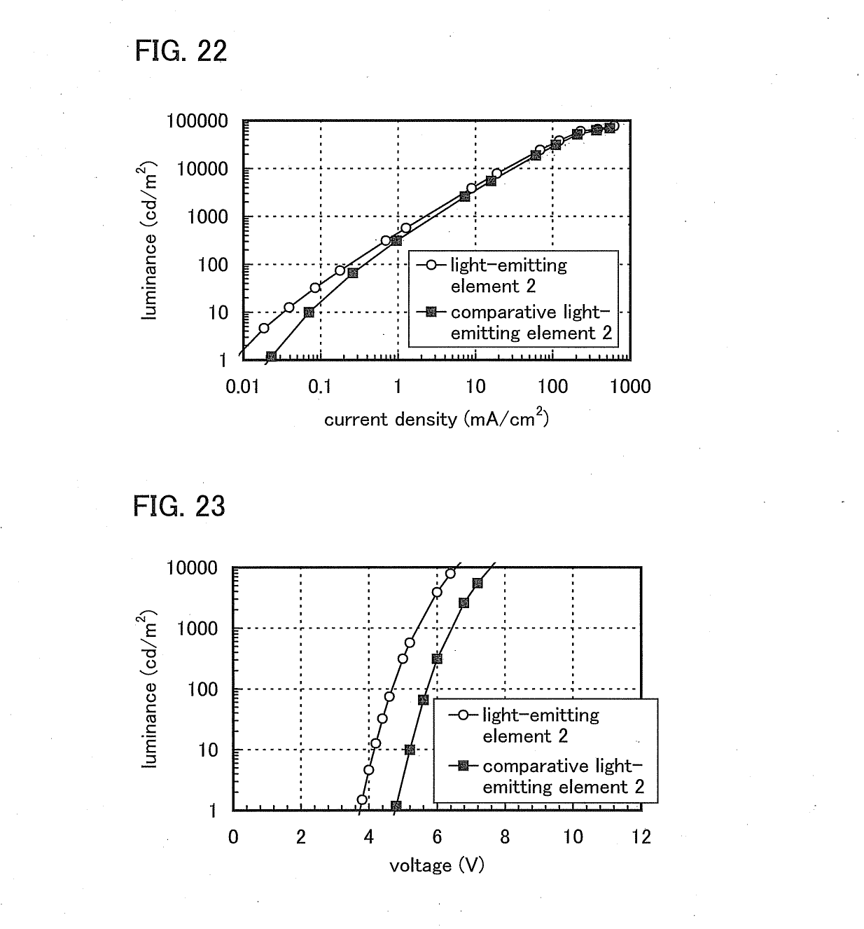

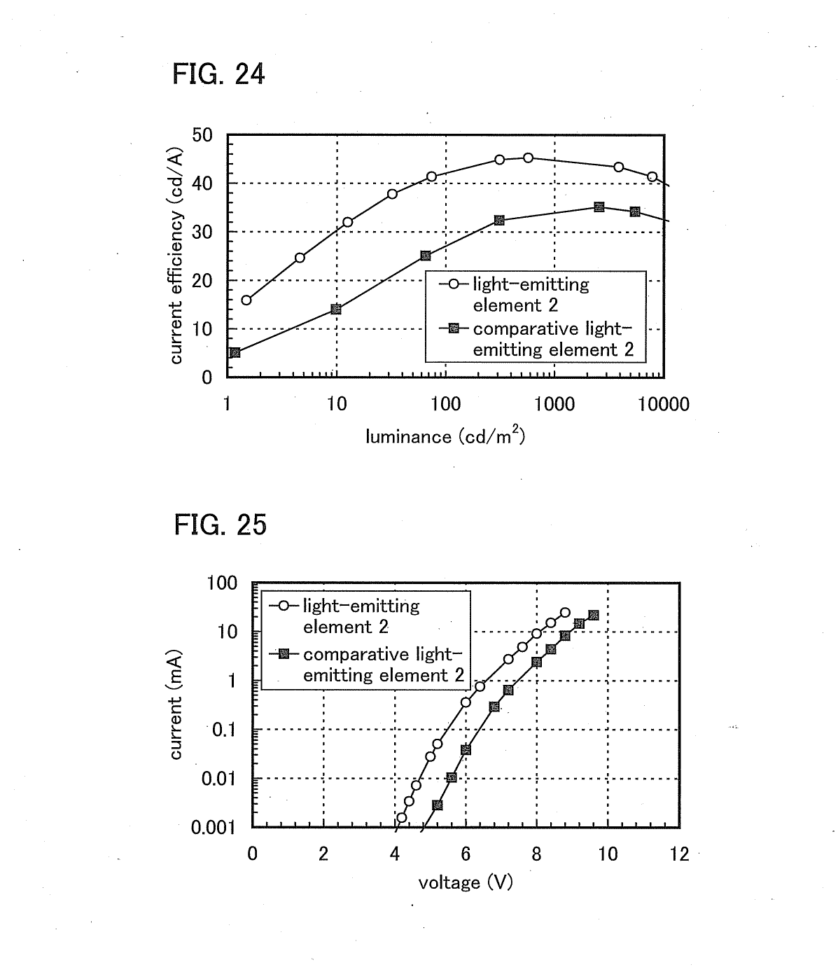

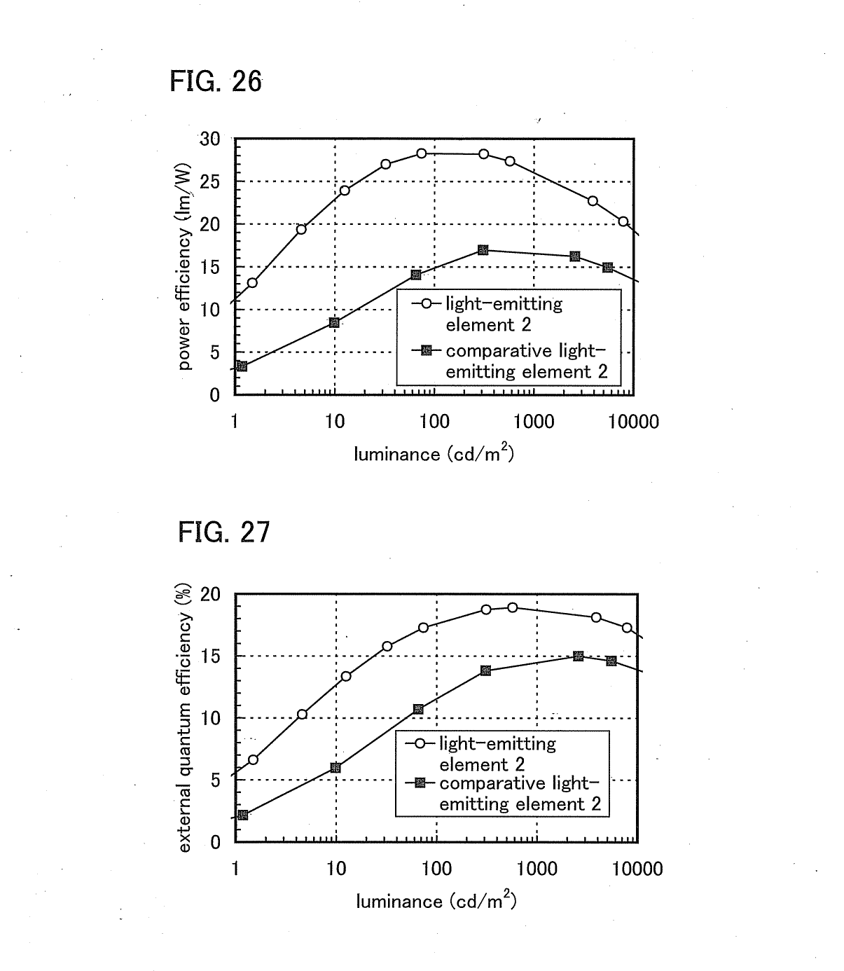

FIG. 22 shows luminance versus current density characteristics of the light-emitting element 2 and the comparative light-emitting element 2. FIG. 23 shows luminance versus voltage characteristics thereof. FIG. 24 shows current efficiency versus luminance characteristics thereof. FIG. 25 shows current versus voltage characteristics thereof FIG. 26 shows power efficiency versus luminance characteristics thereof FIG. 27 shows external quantum efficiency versus luminance characteristics thereof.

In FIG. 22, the vertical axis represents luminance (cd/m²) and the horizontal axis represents current density (mA/cm²). In FIG. 23, the vertical axis represents luminance (cd/m²) and the horizontal axis represents voltage (V). In FIG. 24, the vertical axis represents current efficiency (cd/A) and the horizontal axis represents luminance (cd/m²). In FIG. 25, the vertical axis represents current (mA) and the horizontal axis represents voltage (V). In FIG. 26, the vertical axis represents power efficiency (lm/W) and the horizontal axis represents luminance (cd/m²). In FIG. 27, the vertical axis represents external quantum efficiency (%) and the horizontal axis represents luminance (cd/m²).

FIG. 24 shows that the light-emitting element 2, in which the carbazole compound represented by General Formula (G1) was used as the host material in the light-emitting layer of the light-emitting element emitting blue-green phosphorescence, has better current efficiency versus luminance characteristics and higher emission efficiency than the comparative light-emitting element 2, in which mCP was used as the host material in the same way. This is partly because the carbazole compound represented by General Formula (G1) has as a high triplet excitation level and as a wide energy gap as mCP, which allows even a light-emitting substance emitting blue-green phosphorescence to be effectively excited. In addition, FIG. 23 shows that the light-emitting element, in which the carbazole compound represented by General Formula (G1) was used as the host material in the light-emitting layer of the light-emitting element emitting blue-green phosphorescence, has very good luminance versus voltage characteristics and low driving voltage. This reveals that the carbazole compound represented by General Formula (G1) has a high carrier-transport property. FIG. 22 similarly shows that the light-emitting element 2 has better luminance versus current density characteristics than the comparative light-emitting element 2. Moreover, as shown in FIG. 27, the light-emitting element 2 also has high external quantum efficiency.

As described above, the light-emitting element including the carbazole compound represented by General Formula (G1) has good characteristics such as high emission efficiency and low driving voltage. Thus, as shown in FIG. 26, the power efficiency versus luminance characteristics of the light-emitting element including the carbazole compound represented by General Formula (G1) is much better than those of the comparative light-emitting element.

Note that mCP, which was used for comparison, has a wide energy gap and a high triplet excitation level and thus is often used as a host material in an element emitting short-wavelength phosphorescence and is known to allow a phosphorescent light-emitting element having high emission efficiency to be manufactured. It was found that the carbazole compound described in Embodiment 1 makes it possible to obtain a light-emitting element having much higher emission efficiency than the light-emitting element including mCP.

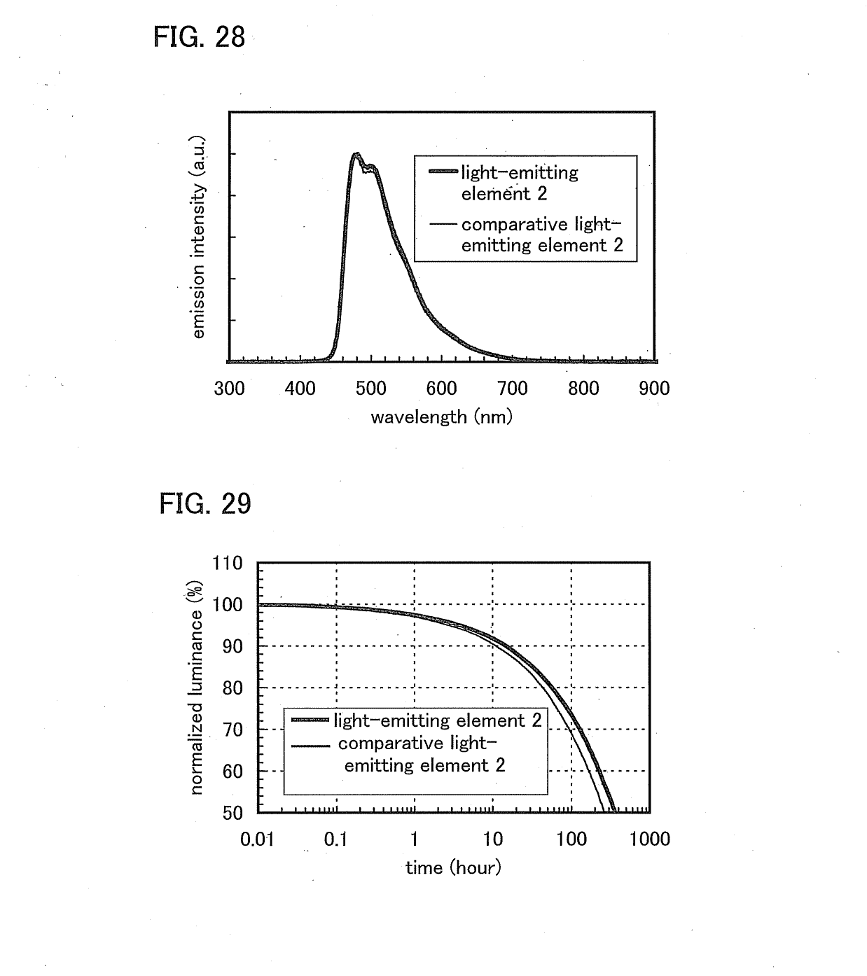

FIG. 28 shows emission spectra of the manufactured light-emitting element 2 and comparative light-emitting element 2 when a current of 0.1 mA was made to flow through each of the light-emitting elements. In FIG. 28, the vertical axis represents emission intensity (arbitrary unit) and the horizontal axis represents wavelength (nm). The emission intensity is shown as a value relative to the maximum emission intensity assumed to be 1. According to FIG. 28, the emission spectra of the light-emitting element 2 and the comparative light-emitting element 2 almost overlap, and both the light-emitting element 2 and the comparative light-emitting element 2 emit blue-green light emanating from [Ir(Mptz)₃], which is the emission center substance.

Next, with an initial luminance set to 300 cd/m², these elements were driven under a condition where the current density was constant, and changes in luminance relative to driving time were examined. FIG. 29 shows normalized luminance versus time characteristics. FIG. 29 shows that the light-emitting element 2 has better characteristics than the comparative light-emitting element 2 and high reliability.

### EXAMPLE 4

In this example, description will be made on a light-emitting element (light-emitting element 3) in which 1-[3,5-di(9H-carbazol-9-yl)phenyl]-2-phenylbenzimidazole (abbreviation: 1Cz2BIm, Structural Formula (100)), the carbazole compound described in Embodiment 1, is used as a host material in a light-emitting layer containing an emission center substance emitting blue phosphorescence.

Molecular structures of organic compounds used in this example are shown in Structural Formulae (i), (ii), (iv) to (vi), (viii), and (100). The element structure was similar to that illustrated in FIG. 1A.

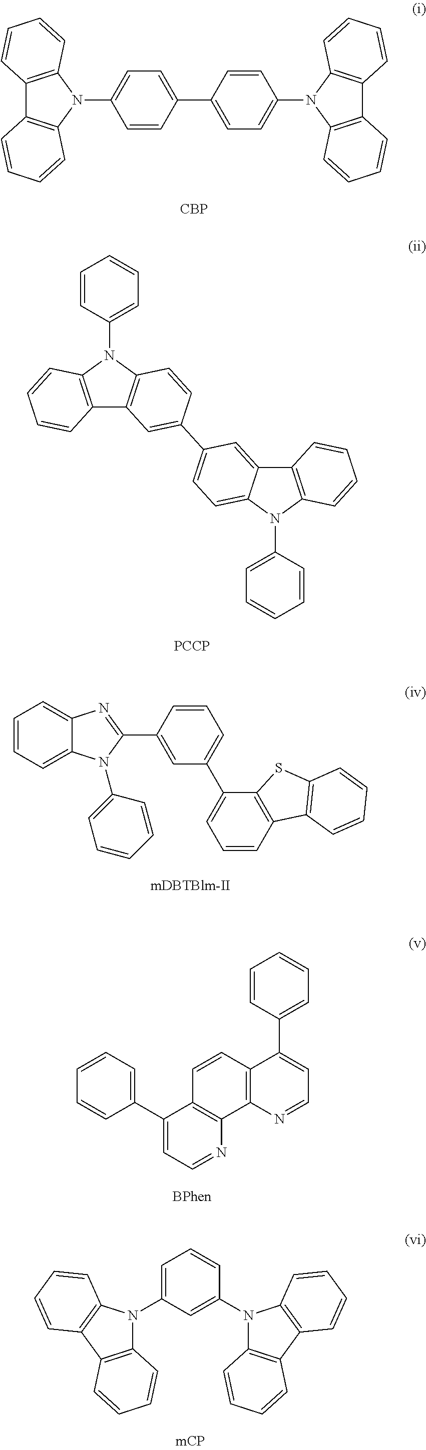

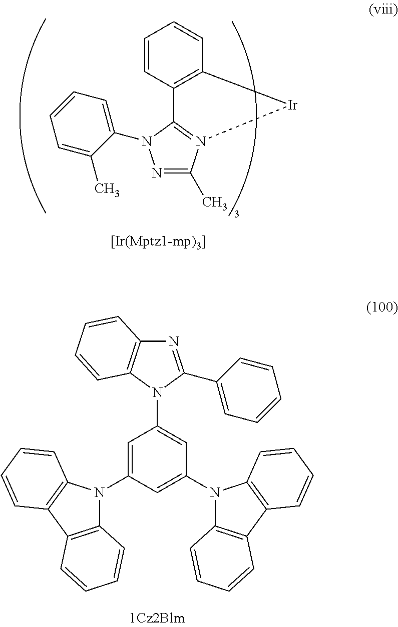

### <<Manufacture of Light-Emitting Element 3 and Comparative Light-Emitting Element 3>>

First, a glass substrate, over which a film of indium tin oxide containing silicon (ITSO) was formed to a thickness of 110 nm as the first electrode 101, was prepared. A surface of the ITSO film was covered with a polyimide film so that an area of 2 mm×2 mm of the surface was exposed. The electrode area was 2 mm×2 mm. As pretreatment for forming the light-emitting element over the substrate, the surface of the substrate was washed with water and baked at 200° C. for 1 hour, and then was subjected to UV ozone treatment for 370 seconds. After that, the substrate was transferred into a vacuum evaporation apparatus where the pressure had been reduced to approximately 10⁻⁴ Pa, and was subjected to vacuum baking at 170° C. for 30 minutes in a heating chamber of the vacuum evaporation apparatus, and then the substrate was cooled down for about 30 minutes.

Next, the substrate was fixed to a holder provided in the vacuum evaporation apparatus so that the surface provided with the ITSO film faced downward.

After the pressure in the vacuum evaporation apparatus was reduced to 10⁻⁴ Pa, 4,4′-bis(N-carbazolyl)biphenyl (abbreviation: CBP) represented by Structural Formula (i) and molybdenum(VI) oxide were deposited by co-evaporation so that the weight ratio of CBP to molybdenum oxide was 2:1, whereby a hole-injection layer 111 was formed. The thickness of the hole-injection layer 111 was 60 nm. Note that co-evaporation is an evaporation method in which a plurality of different substances are vaporized from the respective different evaporation sources at the same time.

Next, a film of 1,3-bis(N-carbazolyl)benzene (abbreviation: mCP) represented by Structural Formula (vi) was formed to a thickness of 20 nm by evaporation, whereby a hole-transport layer 112 was formed.

Further, on the hole-transport layer 112, a film containing 1-[3,5-di(9H-carbazol-9-yl)phenyl]-2-phenylbenzimidazole (abbreviation: 1Cz2BIm) represented by Structural Formula (100), 9-phenyl-9H-3-(9-phenyl-9H-carbazol-3-yl)carbazole (abbreviation: PCCP) represented by Structural Formula and tris [3-methyl-1-(2-methylphenyl)-5-phenyl-1H-1,2,4-triazolato] iridium(III) (abbreviation: [Ir(Mptzl-mp)₃]) represented by Structural Formula (viii) in a 1:0.25:0.06 weight ratio was formed to a thickness of 30 nm by evaporation, and then a film containing 2-[3-(dibenzothiophen-4-yl)phenyl]-1-phenyl-1H-benzimidazole (abbreviation: mDBTBIm-II) represented by Structural Formula (iv) and [Ir(Mptzl-mp)₃] in a 1:0.06 weight ratio was Thinned to a thickness of 10 nm by evaporation to be stacked thereon, whereby a light-emitting layer 113 was formed.

Next, a film of bathophenanthroline (abbreviation: BPhen) represented by Structural Formula (v) was formed to a thickness of 15 nm by evaporation, whereby an electron-transport layer 114 was formed.

Further, a film of lithium fluoride was formed to a thickness of 1 nm on the electron-transport layer 114 by evaporation, whereby an electron-injection layer 115 was formed. Lastly, a film of aluminum was formed to a thickness of 200 nm as a second electrode 102 which serves as a cathode. Thus, the light-emitting element 3 was completed. Note that the above evaporation steps were all performed by a resistance-heating method.

In manufacture of the comparative light-emitting element 3, a light-emitting layer 113 was formed of a stack of a co-evaporation film of mCP and [Ir(Mptzl-mp)₃] and a co-evaporation film of mDBTBIm-II and [Ir(Mptzl-mp)₃], which is different from the light-emitting layer 113 in the light-emitting element 3. In other words, the light-emitting layer 113 in the comparative light-emitting element 3 was formed in such a manner that a co-evaporation film of mCP and [Ir(Mptzl-mp)₃] in a 1:0.06 weight ratio was formed to a thickness of 30 nm, and then a co-evaporation film of mDBTBIm-II and [Ir(Mptzl-mp)₃] in a 1:0.06 weight ratio was formed to a thickness of 10 nm to be stacked thereon.

### <<Operation Characteristics of Light-Emitting Element 3 and Comparative Light-Emitting Element 3>>

The thus obtained light-emitting element 3 and comparative light-emitting element 3 were put in a glove box in a nitrogen atmosphere, and the light-emitting elements were sealed so as not to be exposed to the air. Then, the operation characteristics of these light-emitting elements were measured. Note that the measurement was carried out at room temperature (in an atmosphere kept at 25° C.).

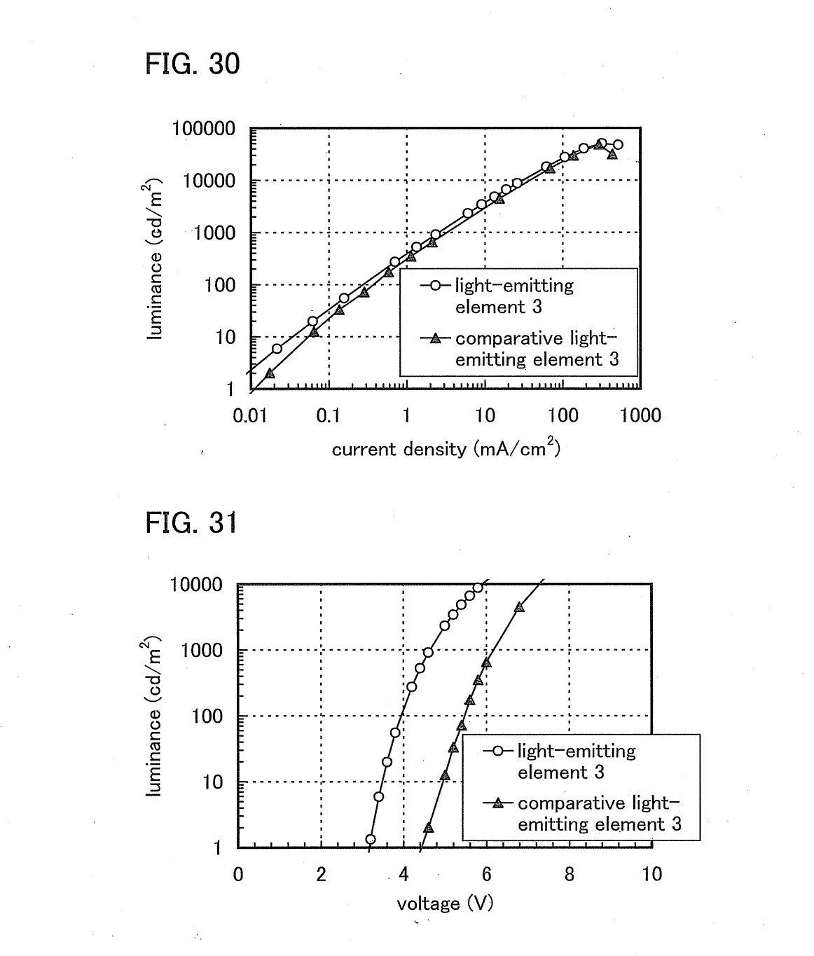

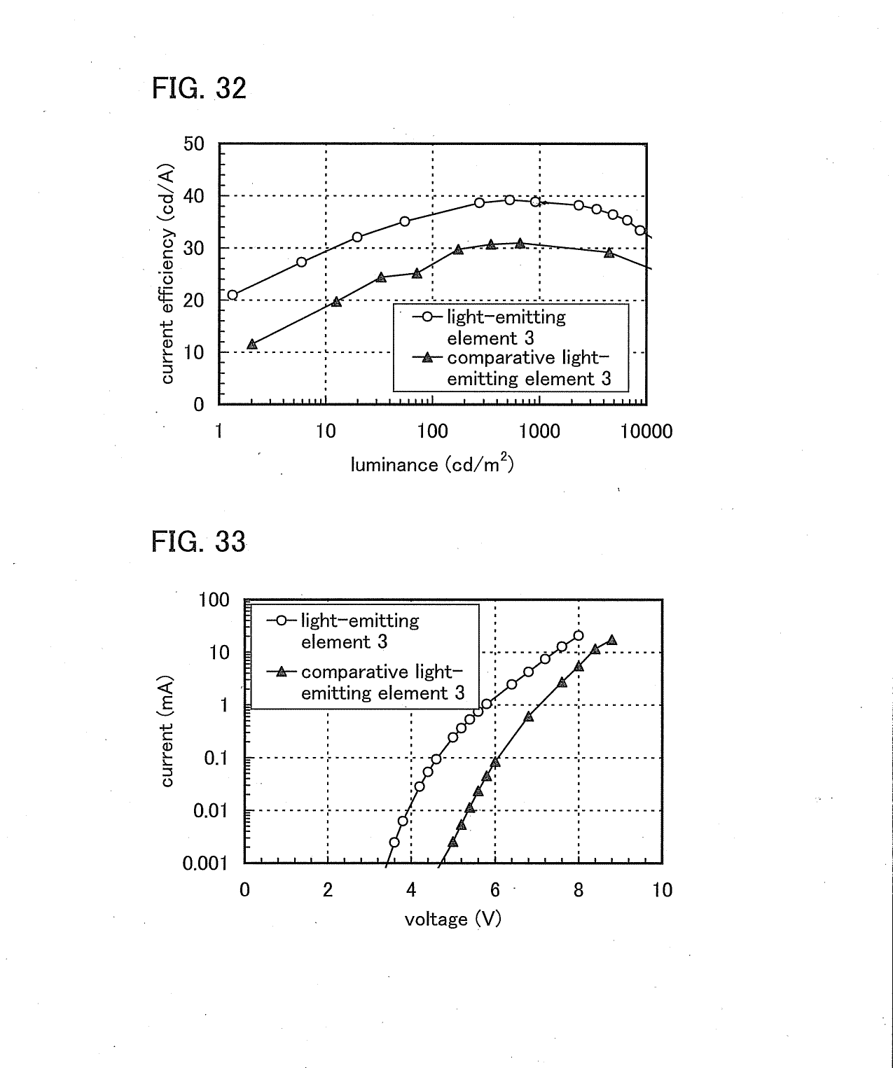

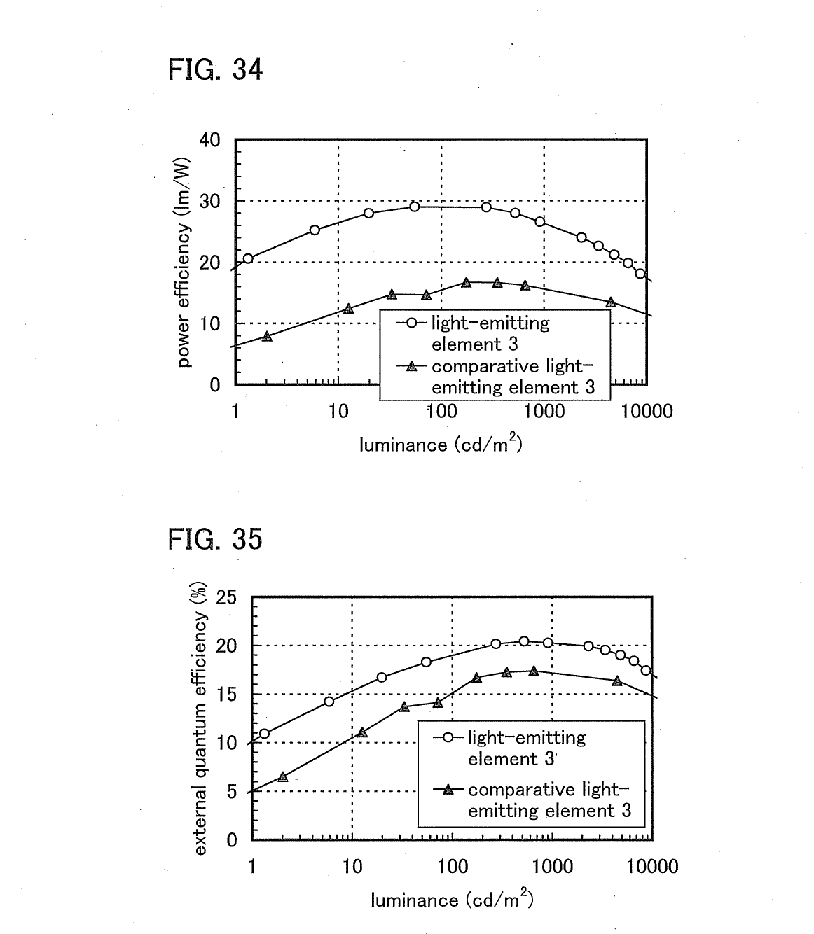

FIG. 30 shows luminance versus current density characteristics of the light-emitting element 3 and the comparative light-emitting element 3. FIG. 31 shows luminance versus voltage characteristics thereof. FIG. 32 shows current efficiency versus luminance characteristics thereof. FIG. 33 shows current versus voltage characteristics thereof. FIG. 34 shows power efficiency versus luminance characteristics thereof. FIG. 35 shows external quantum efficiency versus luminance characteristics thereof.

In FIG. 30, the vertical axis represents luminance (cd/m²) and the horizontal axis represents current density (mA/cm²). In FIG. 31, the vertical axis represents luminance (cd/m²) and the horizontal axis represents voltage (V). In FIG. 32, the vertical axis represents current efficiency (cd/A) and the horizontal axis represents luminance (cd/m²). In FIG. 33, the vertical axis represents current (mA) and the horizontal axis represents voltage (V). In FIG. 34, the vertical axis represents power efficiency (lm/W) and the horizontal axis represents luminance (cd/m²). In FIG. 35, the vertical axis represents external quantum efficiency (%) and the horizontal axis represents luminance (cd/m²).

FIG. 32 shows that the light-emitting element 3, in which the carbazole compound represented by General Formula (G1) was used as the host material in the light-emitting layer of the light-emitting element emitting blue phosphorescence, has better current efficiency versus luminance characteristics and higher emission efficiency than the comparative light-emitting element 3, in which mCP was used as a host material in the same way. This is partly because the carbazole compound represented by General Foimula (G1) has as a high triplet excitation level and as a wide energy gap as mCP, which allows even a light-emitting substance emitting blue phosphorescence to be effectively excited. In addition, FIG. 31 shows that the light-emitting element, in which the carbazole compound represented by General Formula (G1) was used as the host material in the light-emitting layer of the light-emitting element emitting blue phosphorescence, has very good luminance versus voltage characteristics and low driving voltage. This reveals that the carbazole compound represented by General Formula (G1) has a high carrier-transport property. Moreover, as shown in FIG. 35, the light-emitting element 3 also has high external quantum efficiency.

As described above, the light-emitting element including the carbazole compound represented by General Formula (G1) has good characteristics such as high emission efficiency and low driving voltage. Thus, as shown in FIG. 34, the power efficiency versus luminance characteristics of the light-emitting element 3 including the carbazole compound represented by General Formula (G1) is about twice as high as the power efficiency versus luminance characteristics of the comparative light-emitting element 3.

Note that mCP, which was used for comparison, has a wide energy gap and a high triplet excitation level and thus is often used as a host material in an element emitting short-wavelength phosphorescence and is known to allow a phosphorescent light-emitting element having high emission efficiency to be manufactured. It was found that the carbazole compound described in Embodiment 1 makes it possible to obtain a light-emitting element having much higher emission efficiency than the light-emitting element including mCP.

FIG. 36 shows emission spectra of the manufactured light-emitting element 3 and comparative light-emitting element 3 when a current of 0.1 mA was made to flow through each of the light-emitting elements. In FIG. 36, the vertical axis represents emission intensity (arbitrary unit) and the horizontal axis represents wavelength (nm). The emission intensity is shown as a value relative to the maximum emission intensity assumed to be 1. FIG. 36 shows that the light-emitting element 3 and the comparative light-emitting element 3 emit blue light emanating from [Ir(Mptzl-mp)₃], which is the emission center substance.

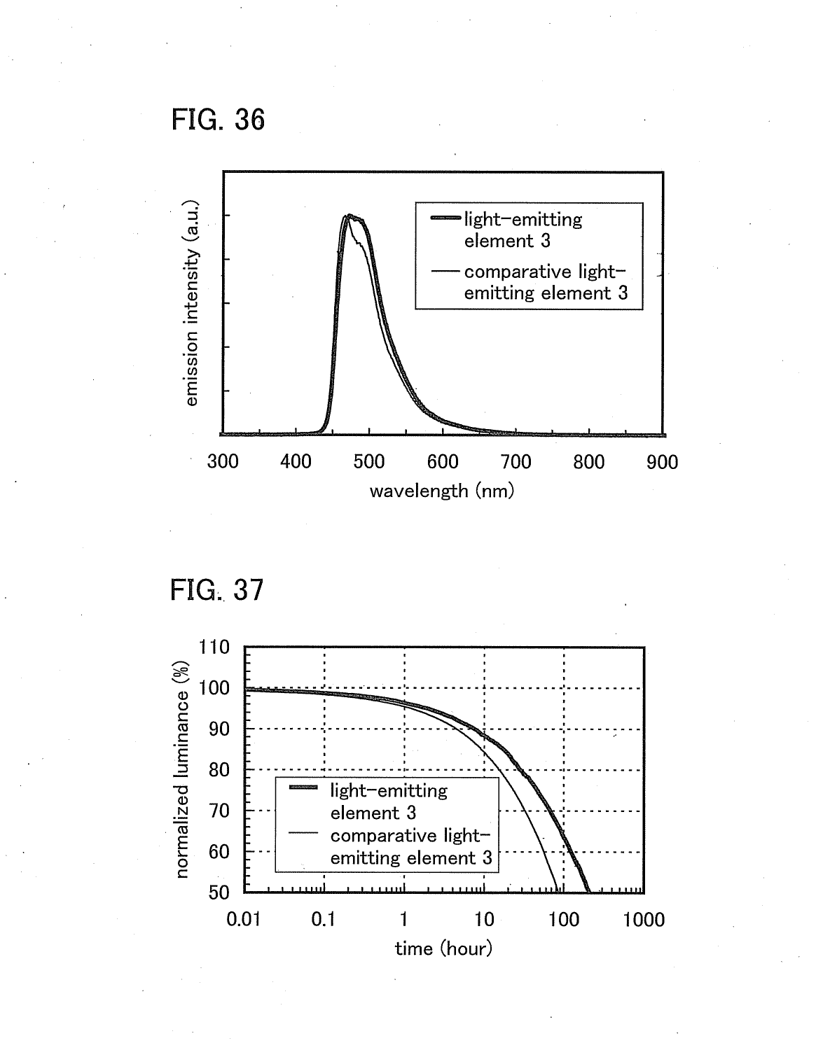

Next, with an initial luminance set to 300 cd/m², these elements were driven under a condition where the current density was constant, and changes in luminance relative to driving time were examined. FIG. 37 shows normalized luminance versus time characteristics. FIG. 37 shows that the light-emitting element 3 has lifetime more than twice as long as that of the comparative light-emitting element 3 (the lifetime here corresponds to the time it takes for the luminance to decrease to half of the initial luminance) and has high reliability.

As described above, the light-emitting element of this embodiment, in which the emission center substance emits blue phosphorescence and the carbazole compound described in Embodiment 1 is used as a host material or a hole-transport material, can have high emission efficiency by efficient excitation for blue phosphorescence which is the light emission from the high triplet excitation level or by prevention of a loss due to energy transfer. This demonstrates that the carbazole compound described in Embodiment 1 has a very high triplet excitation level.

### EXAMPLE 5

In this example, description will be made on a light-emitting element (light-emitting element 4) in which 1-[3,5-di(9H-carbazol-9-yl)phenyl]-2-phenylbenzimidazole (abbreviation: 1Cz2BIm,

Structural Formula (100)), the carbazole compound described in Embodiment 1, is used as a host material in a light-emitting layer containing an emission center substance emitting blue phosphorescence and a light-emitting element (comparative light-emitting element 4) in which N-phenyl-2-[4-(9H-carbazol-9-yl)phenyl]benzimidazole (abbreviation: CzBIm) was used instead of 1Cz2BIm.

Molecular structures of organic compounds used in this example are shown in Structural Formulae (i), (iv) to (vi), (viii), (ix), and (100). The element structure was similar to that illustrated in FIG. 1A.

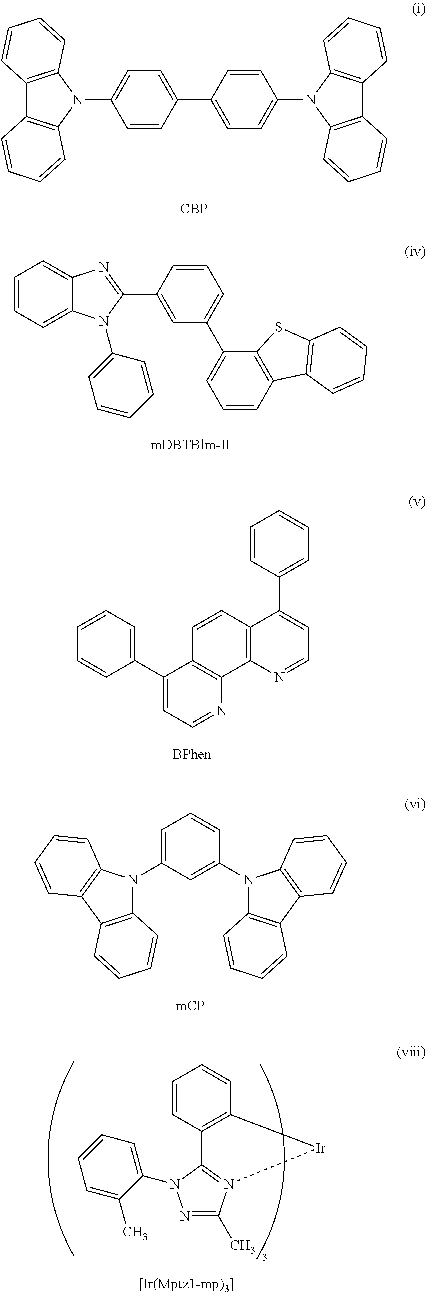

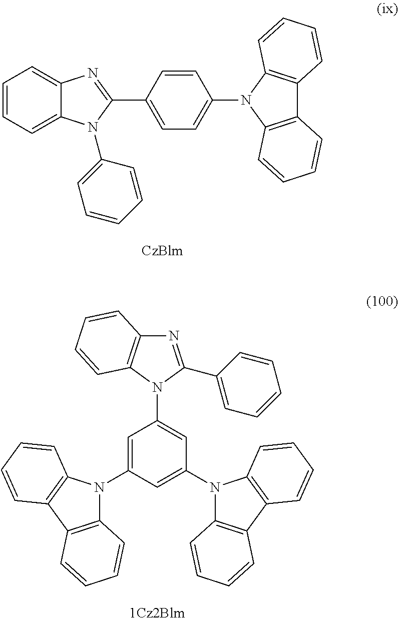 [SMILES: c1ccc(-n2c(-c3ccc(-n4c5ccccc5c5ccccc54)cc3)nc3ccccc32)cc1 ; c1ccc(-c2nc3ccccc3n2-c2cc(-n3c4ccccc4c4ccccc43)cc(-n3c4ccccc4c4ccccc43)c2)cc1]

### <<Manufacture of Light-Emitting Element 4 and Comparative Light-Emitting Element 4>>

First, a glass substrate, over which a film of indium tin oxide containing silicon (ITSO) was formed to a thickness of 110 nm as the first electrode 101, was prepared. A surface of the ITSO film was covered with a polyimide film so that an area of 2 mm×2 mm of the surface was exposed. The electrode area was 2 mm×2 mm. As pretreatment for fat ing the light-emitting element over the substrate, the surface of the substrate was washed with water and baked at 200° C. for 1 hour, and then was subjected to UV ozone treatment for 370 seconds. After that, the substrate was transferred into a vacuum evaporation apparatus where the pressure had been reduced to approximately 10⁻⁴ Pa, and was subjected to vacuum baking at 170° C. for 30 minutes in a heating chamber of the vacuum evaporation apparatus, and then the substrate was cooled down for about 30 minutes.

Next, the substrate was fixed to a holder provided in the vacuum evaporation apparatus so that the surface provided with the ITSO film faced downward.

After the pressure in the vacuum evaporation apparatus was reduced to 10⁻⁴ Pa, 4,4′-bis(N-carbazolyl)biphenyl (abbreviation: CBP) represented by Structural Forumla (i) and molybdenum(VI) oxide were deposited by co-evaporation so that the weight ratio of CBP to molybdenum oxide was 2:1, whereby a hole-injection layer 111 was formed. The thickness of the hole-injection layer 111 was 60 nm. Note that co-evaporation is an evaporation method in which a plurality of different substances are vaporized from the respective different evaporation sources at the same time.

Next, a film of 1,3-bis(N-carbazolyl)benzene (abbreviation: mCP) which is represented by Structural Formula (vi) was formed to a thickness of 20 nm by evaporation, whereby a hole-transport layer 112 was formed.

Further, on the hole-transport layer 112, a film containing 1-[3,5-di(9H-carbazol-9-yl)phenyl]-2-phenylbenzimidazole (abbreviation: 1Cz2BIm) represented by Structural Formula (100) and tris[3-methyl-1-(2-methylphenyl)-5-phenyl-1H-1,2,4-triazolato]iridium(III) (abbreviation: [Ir(Mptzl-mp)₃]) represented by Structural Formula (viii) in a 1:0.08 weight ratio was formed to a thickness of 30 nm by evaporation, and then a film containing 2-[3-(dibenzothiophen-4-yl)phenyl]-1-phenyl-1H-benzimidazole (abbreviation: mDBTBIm-II) represented by Structural Formula (iv) and [Ir(Mptzl-mp)₃] in a 1:0.08 weight ratio was formed to a thickness of 10 mn by evaporation to be stacked thereon, whereby a light-emitting layer 113 was formed.

Next, a film of bathophenanthroline (abbreviation: BPhen) represented by Structural Formula (v) was formed to a thickness of 15 nm by evaporation, whereby an electron-transport layer 114 was formed.

Further, a film of lithium fluoride was formed to a thickness of 1 nm on the electron-transport layer 114 by evaporation, whereby an electron-injection layer 115 was formed. Lastly, a film of aluminum was formed to a thickness of 200 nm as a second electrode 102 which serves as a cathode. Thus, the light-emitting element 4 was completed. Note that the above evaporation steps were all performed by a resistance-heating method.

In manufacture of the comparative light-emitting element 4, a light-emitting layer 113 was foinied of a stack of a co-evaporation film of N-phenyl-2-[4-(9H-carbazol-9-yl)phenyl]benzimidazole (abbreviation: CzBIm) represented by Structural Formula (ix) and [Ir(Mptzl-mp)₃] and a co-evaporation film of mDBTBIm-ll and [Ir(Mptzl-mp)₃], which is different from the light-emitting layer 113 in the light-emitting element 4. In other words, the light-emitting layer 113 in the comparative light-emitting element 4 was formed in such a manner that a co-evaporation film of CzBIm and [Ir(Mptzl-mp)₃] in a 1:0.08 weight ratio was formed to a thickness of 30 nm, and then a co-evaporation film of mDBTBIm-II and [Ir(Mptzl-mp)₃] in a 1:0.08 weight ratio was formed to a thickness of 10 nm to be stacked thereon.

### <<Operation Characteristics of Light-Emitting Element 4 and Comparative Light-Emitting Element 4>>

The thus obtained light-emitting element 4 and comparative light-emitting element 4 were put in a glove box in a nitrogen atmosphere, and the light-emitting elements were sealed so as not to be exposed to the air. Then, the operation characteristics of these light-emitting elements were measured. Note that the measurement was carried out at room temperature (in an atmosphere kept at 25° C.).

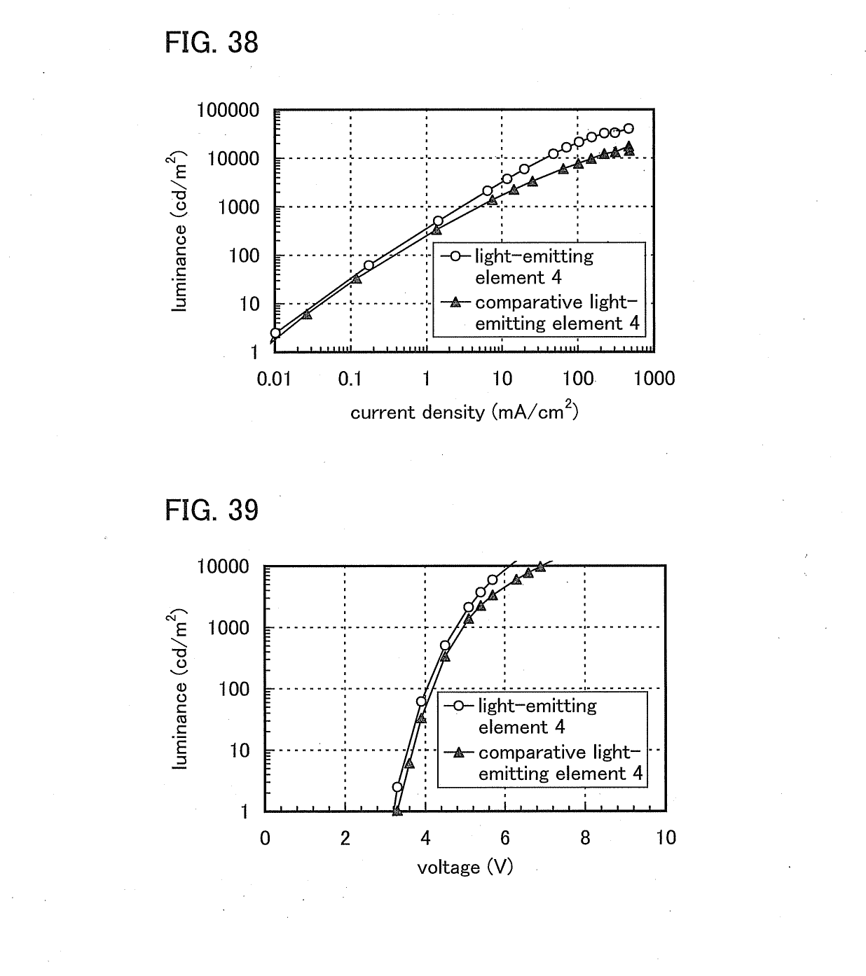

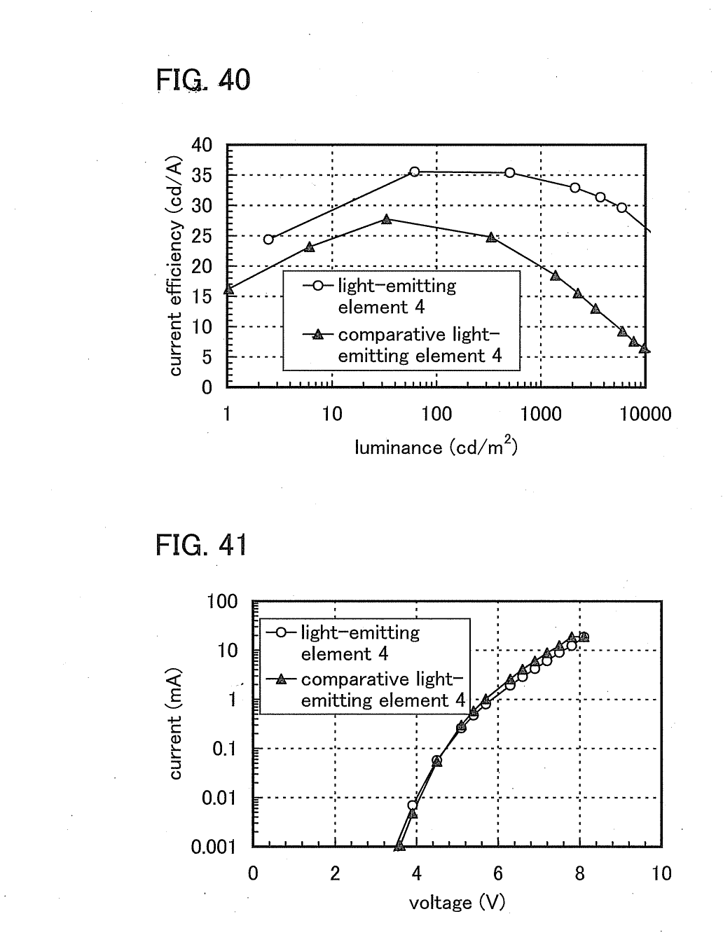

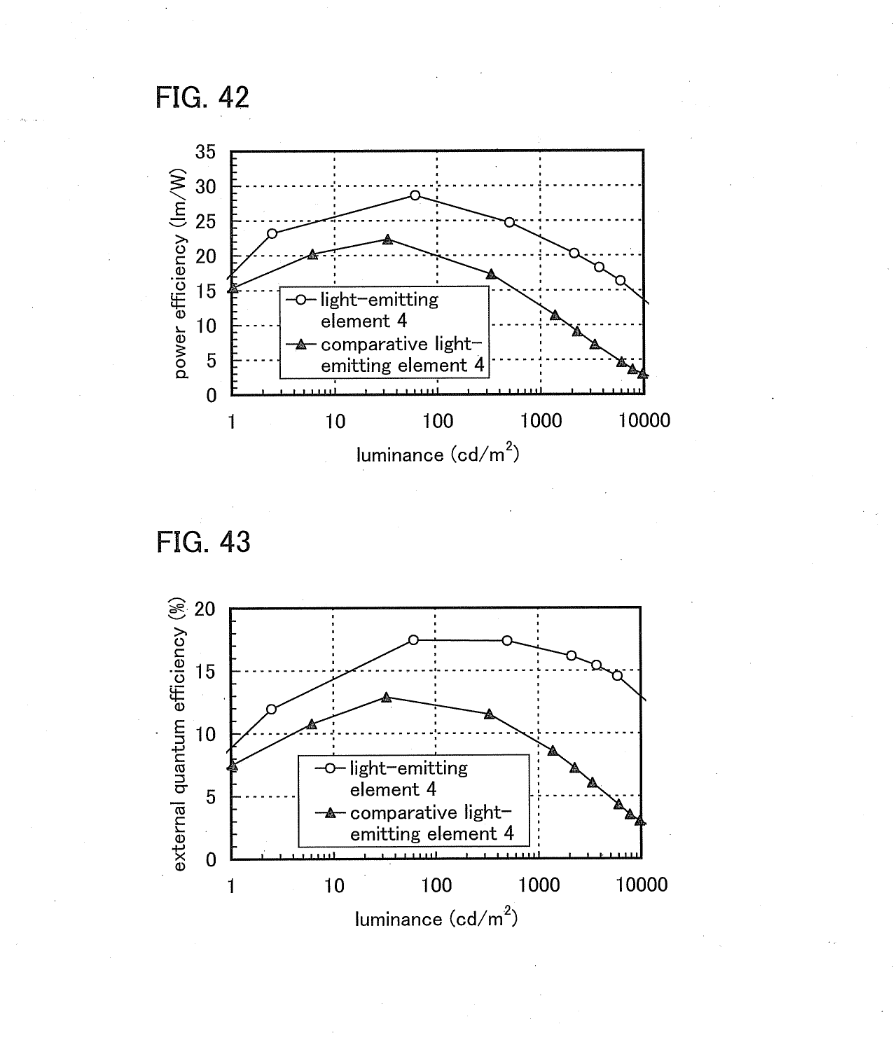

FIG. 38 shows luminance versus current density characteristics of the light-emitting element 4 and the comparative light-emitting element 4. FIG. 39 shows luminance versus voltage characteristics thereof FIG. 40 shows current efficiency versus luminance characteristics thereof FIG. 41 shows current versus voltage characteristics thereof. FIG. 42 shows power efficiency versus luminance characteristics thereof FIG. 43 shows external quantum efficiency versus luminance characteristics thereof

In FIG. 38, the vertical axis represents luminance (cd/m²) and the horizontal axis represents current density (mA/cm²). In FIG. 39, the vertical axis represents luminance (cd/m²) and the horizontal axis represents voltage (V). In FIG. 40, the vertical axis represents current efficiency (cd/A) and the horizontal axis represents luminance (cd/m²). In FIG. 41, the vertical axis representss current (mA) and the horizontal axis represents voltage (V). In FIG. 42, the vertical axis represents power efficiency (lm/W) and the horizontal axis represents luminance (cd/m²). In FIG. 43, the vertical axis represents external quantum efficiency (%) and the horizontal axis represents luminance (cd/m²).

FIG. 40 shows that the light-emitting element 4, in which the carbazole compound represented by General Formula (G1) was used as the host material in the light-emitting layer of the light-emitting element emitting blue phosphorescence, has better current efficiency versus luminance characteristics and higher emission efficiency than the comparative light-emitting element 4, in which CzBIm was used as a host material in the same way. This is partly because the carbazole compound represented by General Formula (G1) has a high triplet excitation level and a wide energy gap, which allows even a light-emitting substance emitting blue phosphorescence to be effectively excited. In addition, FIG. 39 shows that the light-emitting element, in which the carbazole compound represented by General Formula (G1) was used as the host material in the light-emitting layer of the light-emitting element emitting blue phosphorescence, has very good luminance versus voltage characteristics and low driving voltage. This reveals that the carbazole compound represented by General Formula (G1) has a high carrier-transport property. Moreover, as shown in FIG. 43, the light-emitting element 4 also has high external quantum efficiency. .

As described above, the light-emitting element including the carbazole compound represented by General Formula (G1) has good characteristics such as high emission efficiency and low driving voltage. Thus, as shown in FIG. 42, the power efficiency versus luminance characteristics of the light-emitting element 4 including the carbazole compound represented by General Formula (G1) is twice as high as the power efficiency versus luminance characteristics of the comparative light-emitting element 4.

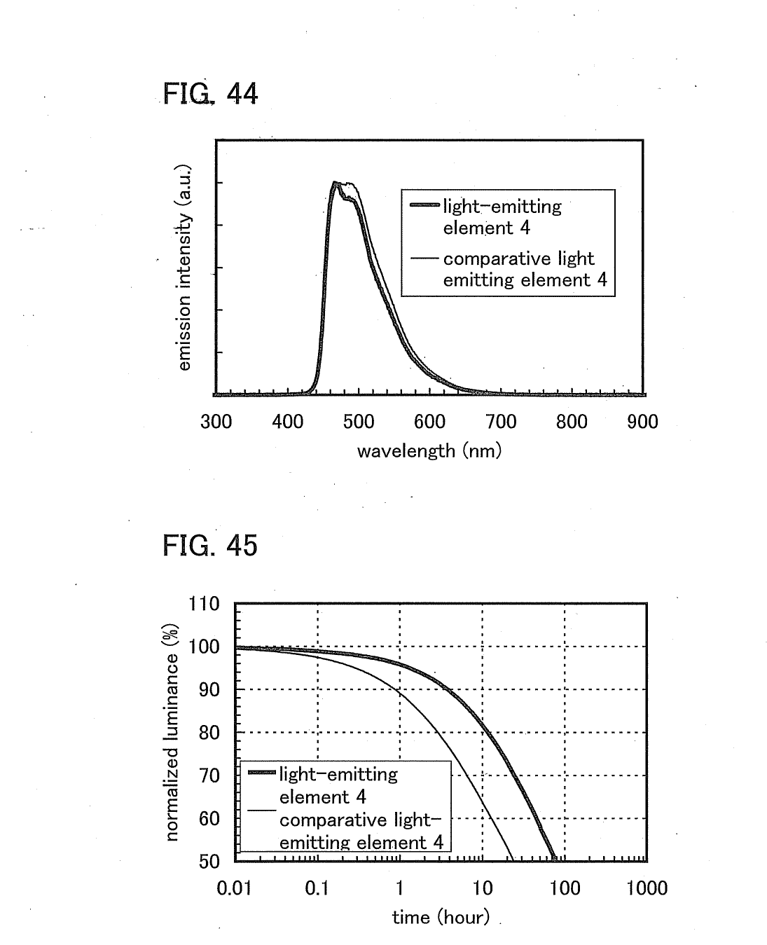

FIG. 44 shows emission spectra of the manufactured light-emitting element 4 and the comparative light-emitting element 4 when a current of 0.1 mA was made to flow through each of the light-emitting elements. In FIG. 44, the vertical axis represents emission intensity (arbitrary unit) and the horizontal axis represents wavelength (nm). The emission intensity is shown as a value relative to the maximum emission intensity assumed to be 1. According to FIG. 44, the light-emitting element 4 and the comparative light-emitting element 4 each emit blue light emanating from [Ir(Mptzl-mp)₃], which is the emission center substance.

Next, with an initial luminance set to 300 cd/m², these elements were driven under a condition where the current density was constant, and changes in luminance relative to driving time were examined. FIG. 45 shows normazlied luminance versus time characteristics. FIG. 45 shows that the light-emitting element 4 has lifetime more than twice as long as that of the comparative light-emitting element 4 (the lifetime here corresponds to the time it takes for the luminance to decrease to half of the initial luminance) and has high reliability.

As described above, the light-emitting element of this embodiment, in which the emission center substance emits blue phosphorescence and the carbazole compound described in Embodiment 1 is used a host material or as a hole-transport material, can have high emission efficiency by efficient excitation for blue phosphorescence which is the light emission from the high triplet excitation level or by prevention of a loss due to energy transfer. This demonstrates that the carbazole compound described in Embodiment 1 has a very high triplet excitation level.

### REFERENCE EXAMPLE

In this reference example, materials used in Examples will be described.

### <Synthesis Example of [Ir(Mptz)₃]

A synthesis example of tris(5-methyl-3,4-diphenyl-4H-1,2,4-triazolato)iridium(III) (abbreviation: [Ir(Mptz)₃]), which was used in Example 3, will be described.

### Step 1: Synthesis of 3-Methyl-4,5-diphenyl-4H-1,2,4-triazole (abbreviation: HMptz)

First, 5.04 g of thioacetanilide, 5.44 g of benzoylhydrazine, and 50 mL of 1-butanol were put in a round-bottom flask provided with a reflux pipe, and the air in the flask was replaced with argon. This reaction container was irradiated with microwaves (2.45 GHz, 100 W) for 2 hours and 45 minutes to be heated. Then, water was added to this solution and the organic layer was extracted with dichloromethane. The obtained organic layer was washed with water and dried with magnesium sulfate. After the drying, the solution was filtrated. The solvent of this solution was distilled off, and the resulting residue was purified by silica gel column chromatography using ethyl acetate as a developing solvent, so that 3-methyl-4,5-diphenyl-4H-1,2,4-triazole (abbreviation: HMptz) was obtained (pale yellow powder, yield of 18%). A scheme of the synthesis of Step 1 is shown below.

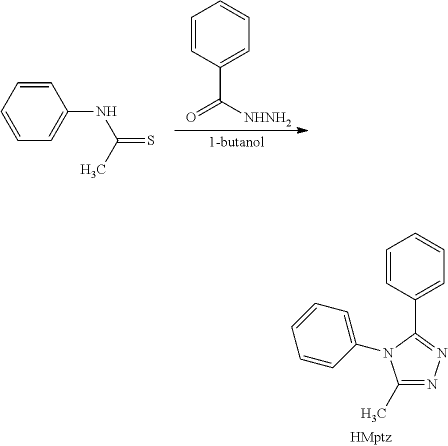

### Step 2: Synthesis of Tris(5-methyl-3,4-diphenyl-4H-1,2,4-triazolato)iridium(III) (abbreviation: [Ir(Mptz)₃)

Next, 1.40 g of the ligand HMptz obtained in Step 1 and 0.58 g of tris(acetylacetonato)iridium(III) were put in a reaction container provided with a three-way cock, and the air in the reaction container was replaced with argon. Then, the mixture was heated at 250° C. for 17 hours and 30 minutes to cause a reaction. The reactant was dissolved in dichloromethane, and this solution was filtered. The solvent of the resulting filtrate was distilled off, and purification was conducted by silica gel column chromatography using ethyl acetate as a developing solvent. Further, recrystallization from a mixed solvent of dichloromethane and hexane was performed, so that the organometallic complex [Ir(Mptz)₃], which is one embodiment of the present invention, was obtained (yellow powder, yield of 22%). A scheme of the synthesis of Step 2 is shown below.

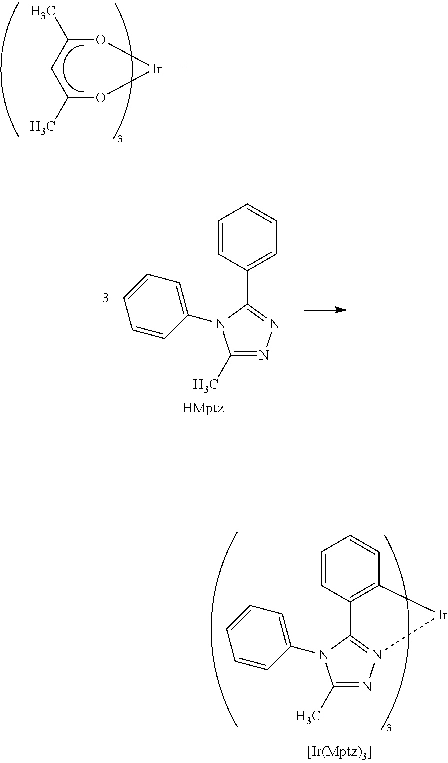

Analysis results by nuclear magnetic resonance spectroscopy (¹H-NMR) of the yellow powder obtained in Step 2 are shown below. These results reveal that the organometallic complex [Ir(Mptz)₃] was obtained.

¹H-NMR. δ(CDCl₃): 2.17 (s, 9H), 6.38 (d, 3H), 6.54 (t, 3H), 6.72 (dt, 3H), 6.87 (dd, 3H), 7.34 (m, 3H), 7.51 (brm, 3H), 7.57 (m, 9H).

### <Synthesis Example of [Ir(Mptzl-mp)₃]>

A synthesis example of tris [3-methyl-1-(2-methylphenyl)-5-phenyl-1H-1,2,4-triazolato]iridium(III) (abbreviation: [Ir(Mptzl-mp)₃]), which was used in Example 4 and Example 5, will be described.

### Step 1: Synthesis of N-(1-Ethoxyethylidene)benzamide

First, 15.5 g of ethyl acetimidate hydrochloride, 150 mL of toluene, and 31.9 g of triethylamine (Et₃N) were put in a 500 mL three-neck flask and stirred at room temperature for. 10 minutes. With a 50-mL dropping funnel, a mixed solution of 17.7 g of benzoyl chloride and 30 mL of toluene were added dropwise to this mixture, and the mixture was stirred at room temperature for 24 hours. After a predetermined time elapsed, the reaction mixture was suction-filtered, and the solid was washed with toluene. The obtained filtrate was concentrated to give N-(1-ethoxyethylidene)benzamide (red oily substance, yield of 82%). A scheme of the synthesis of Step 1 is shown below.

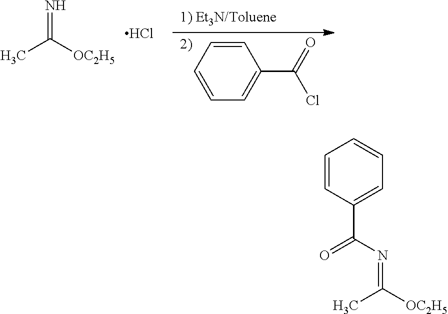

### Step 2: Synthesis of 3-Methyl-1-(2-methylphenyl)-5-phenyl-1H-1,2,4-triazole (abbreviation: HMptzl-mp)

Next, 8.68 g of o-tolyl hydrazine hydrochloride, 100 mL of carbon tetrachloride, and 35 mL of triethylamine (Et₃N) were put in a 300 mL recovery flask, and the mixture was stirred at room temperature for 1 hour. After a predetermined time elapsed, 8.72 g of N-(1-ethoxyethylidene)benzamide obtained in the Step 1 was added to this mixture, and the mixture was stirred at room temperature for 24 hours. After a predetermined time elapsed, water was added to the reaction mixture, and the aqueous layer was subjected to extraction with chloroform. The organic layer was washed with saturated saline, and dried with anhydrous magnesium sulfate added thereto. The obtained mixture was gravity-filtered, and the filtrate was concentrated to give an oily substance. The given oily substance was purified by silica gel column chromatography. As a developing solvent, dichloromethane was used. The obtained fraction was concentrated to give 3-methyl-1-(2-methylphenyl)-5-phenyl-1H-1,2,4-triazole (abbreviation: HMptzl-mp) (orange oily substance, yield of 84%). A scheme of the synthesis of Step 2 is shown below.

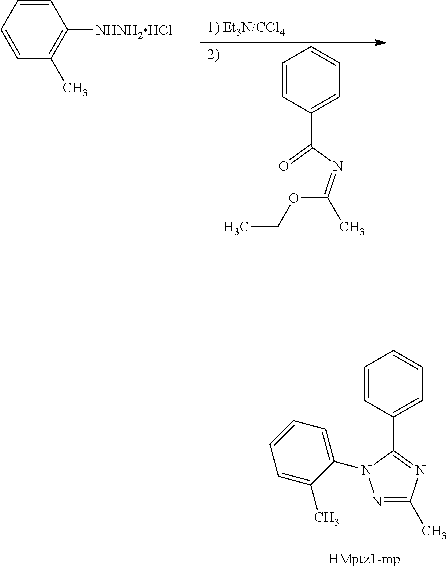

### Step 3: Synthesis of Tris[3-methyl-1-(2-methylphenyl)-5-phenyl-1H-1,2,4-triazolato]iridium(III) (abbreviation: [Ir(Mptzl-mp)₃])]

Next, 2.71 g of the ligand HMptzl-mp obtained in Step 2 and 1.06 g of tris(acetylacetonato)iridium(III) were put in a reaction container provided with a three-way cock. The air in the reaction container was replaced with argon, and the mixture was heated at 250° C. for 48 hours to cause a reaction. This reaction mixture was dissolved in dichloromethane and purified by silica gel column chromatography. As a developing solvent, dichloromethane was used first, and then a mixed solvent of dichloromethane and ethyl acetate in a ratio of 10:1 (v/v) was used. The obtained fraction was concentrated to give a solid. This solid was washed with ethyl acetate, and recrystallized from a mixed solvent of dichloromethane and ethyl acetate to give an organometallic complex [Ir(Mptzl-mp)₃] (yellow powder, yield of 35%). A scheme of the synthesis of Step 3 is shown below.

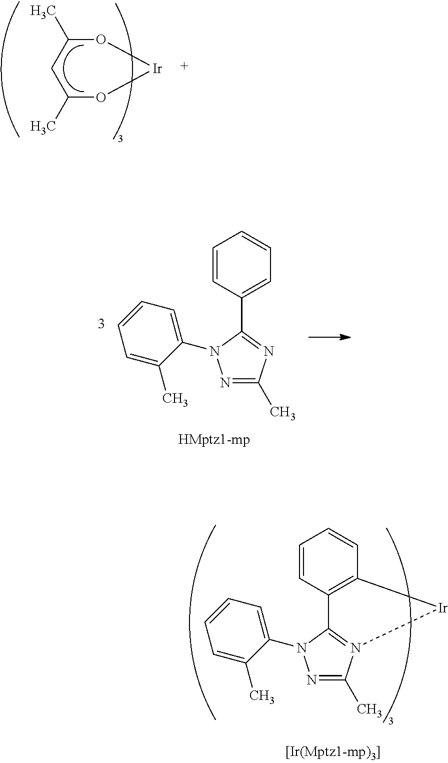

Analysis results of the yellow powder obtained in Step 3 by nuclear magnetic resonance spectrometry (¹H NMR) are shown below. The results reveal that [Ir(Mptzl-mp)₃] was obtained.

¹H NMR data of the obtained substance are as follows: ¹H NMR. δ(CDCl₃): 1.94-2.21 (m, 18H), 6.47-6.76 (m, 12H), 7.29-7.52 (m, 12H).

This application is based on Japanese Patent Application serial no. 2011-207410 filed with Japan Patent Office on Sep. 22, 2011, the entire contents of which are hereby incorporated by reference.

What is claimed is:

1. A carbazole compound represented by General Formula (G1): 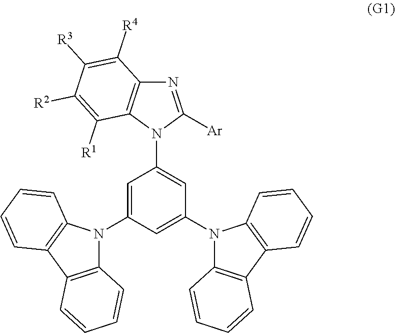
wherein Ar represents an aryl group having 6 to 18 carbon atoms,
wherein R¹ to R⁴ represents any of hydrogen, an alkyl group having 1 to 4 carbon atoms, and an aryl group having 6 to 13 carbon atoms.

2. A light-emitting element material comprising the carbazole compound according to claim 1.

3. A light-emitting element comprising:
a layer containing an organic compound between a pair of electrodes,
wherein the layer containing an organic compound contains the carbazole compound according to claim 1, and
wherein light is emitted by applying current between the pair of electrodes.

4. A light-emitting element comprising:
a layer containing an organic compound between a pair of electrodes,
wherein the layer containing an organic compound comprises a light-emitting layer containing a host material and an emission center material,
wherein the carbazole compound according to claim 1 is contained as the host material, and
wherein light is emitted by applying current between the pair of electrodes.

5. A light-emitting device comprising the light-emitting element according to claim 3.

6. A light-emitting device comprising the light-emitting element according to claim 4.

7. A lighting device comprising the light-emitting element according to claim 3.

8. A lighting device comprising the light-emitting element according to claim 4.

9. A display device comprising the light-emitting element according to claim 3.

10. A display device comprising the light-emitting element according to claim 4.

11. An electronic device comprising the light-emitting element according to claim 3

12. An electronic device comprising the light-emitting element according to claim 4.

13. A carbazole compound represented by Structural Formula (100):

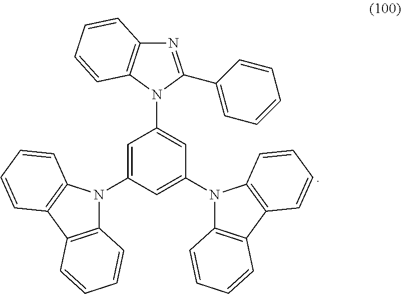 [SMILES: c1ccc(-c2nc3ccccc3n2-c2cc(-n3c4ccccc4c4ccccc43)cc(-n3c4ccccc4c4ccccc43)c2)cc1]

14. A light-emitting element material comprising the carbazole compound according to claim 13.

15. A light-emitting element comprising:
a layer containing an organic compound between a pair of electrodes,
wherein the layer containing an organic compound contains the carbazole compound according to claim 13, and
wherein light is emitted by applying current between the pair of electrodes.

16. A light-emitting element comprising:
a layer containing an organic compound between a pair of electrodes,
wherein the layer containing an organic compound comprises a light-emitting layer containing a host material and an emission center material,
wherein the carbazole compound according to claim 13 is contained as the host material, and
wherein light is emitted by applying current between the pair of electrodes.

17. A light-emitting device comprising the light-emitting element according to claim 15.

18. A light-emitting device comprising the light-emtting element according to claim 16.

19. A lighting device comprising the light-emitting element according to claim 15.

20. A lighting device comprising the light-emitting element according to claim 16.

21. A display device comprising the light-emitting element according to claim 15.

22. A display device comprising the light-emitting element according to claim 16.

23. An electronic device comprising the light-emitting element according to claim 15.

24. An electronic device comprising the light-emitting element according to claim 16.
